(19) United States
(12) Patent Application Publication
(10) Pub. No.: US 2023/0000962 A1
(43) Pub. Date: Jan. 5, 2023

(54) TREATMENT INVOLVING IMMUNE EFFECTOR CELLS GENETICALLY MODIFIED TO EXPRESS ANTIGEN RECEPTORS

(71) Applicant: BIONTECH CELL & GENE THERAPIES GMBH, Mainz (DE)

(72) Inventors: Arne BILLMEIER, Mainz (DE); Matthias BIRTEL, Mainz (DE); Nina HAYDUK, Mainz (DE); Veronika JAHNDEL, Mainz (DE); Oliver KLEIN, Mainz (DE); Kathrin KUNA, Mainz (DE); Kristina MICHEL, Mainz (DE); Petra OEHM, Mainz (DE); Yasmina OUCHAN, Mainz (DE); Katharina REINHARD, Mainz (DE); Benjamin RENGSTL, Mainz (DE); Ugur SAHIN, Mainz (DE)

(21) Appl. No.: 17/757,195

(22) PCT Filed: Dec. 22, 2020
(86) PCT No.: PCT/EP2020/087623

§ 371 Date: Jun. 10, 2022

(30) Foreign Application Priority Data
Dec. 23, 2019 (EP) PCT/EP2019/086950

(51) Int. Cl. A61K 39/00; A61K 35/17; A61P 35/00

(52) U.S. Cl. CPC A61K 39/0011; A61K 35/17; A61P 35/00; A61K 2039/53; A61K 2039/5158; A61K 2039/6018

(57) ABSTRACT

The present disclosure relates to methods for enhancing the efficiency of therapies involving immune effector cells such as T cells engineered to express antigen receptors such as T cell receptors (TCRs) or chimeric antigen receptors (CARs). It is demonstrated herein that such antigen receptor-engineered immune effector cells, even when provided to a subject in sub-therapeutic amounts, are extremely effective in the treatment of cancer diseases, even those cancer diseases that are known to be difficult to treat with antigen receptor-engineered immune effector cells, such as solid tumors or cancers, if additionally target antigen for the antigen receptor is provided to the subject. Immune effector cells may be engineered ex vivo or in vitro and subsequently the immune effector cells may be administered to a subject in need of treatment, or immune effector cells may be engineered in vivo in a subject in need of treatment.

### TECHNICAL FIELD

The present disclosure relates to methods for enhancing the efficiency of therapies involving immune effector cells such as T cells engineered to express antigen receptors such as T cell receptors (TCRs) or chimeric antigen receptors (CARs). In one embodiment, the immune effector cells are genetically modified to express the antigen receptor. Such genetic modification may be effected ex vivo or in vitro and subsequently the immune effector cells may be administered to a subject in need of treatment, or may be effected in vivo in a subject in need of treatment. These methods are, in particular, useful for the treatment of solid cancers characterized by diseased cells expressing an antigen the antigen receptor is directed to. It is demonstrated herein that such antigen receptor-engineered immune effector cells, even when provided to a subject in sub-therapeutic amounts, are extremely effective in the treatment of cancer diseases, even those cancer diseases that are known to be difficult to treat with antigen receptor-engineered immune effector cells, such as solid tumors or cancers, if additionally target antigen for the antigen receptor is provided to the subject. In one embodiment, the immune effector cells by means of an antigen receptor such as T cell receptor (TCR) or chimeric antigen receptor (CAR) bind to the antigen or a procession product thereof when present on or presented in the context of MHC by cells of secondary lymphoid organs such as antigen presenting cells, in particular dendritic cells. The antigen receptor-engineered immune effector cells may be provided to a subject by administering the antigen receptor-engineered immune effector cells or by generating the antigen receptor-engineered immune effector cells in the subject. In one embodiment, the antigen receptor-engineered immune effector cells are generated in the subject treated. Such in vivo generation generally will only provide small amounts of antigen receptor-engineered immune effector cells in the subject. However, it is expected that these small amounts of antigen receptor-engineered immune effector cells will be therapeutically effective due to the strong stimulatory effect achieved by provision of target antigen for the antigen receptor. The target antigen for the antigen receptor may be provided to a subject by administering to the subject an antigen targeted by the antigen receptor, a polynucleotide encoding the antigen, or cells expressing the antigen. The antigen to which the antigen receptor is targeted may comprise a naturally occurring antigen or a variant thereof, or a fragment of the naturally occurring antigen or variant thereof. In one particularly preferred embodiment, the polynucleotide encoding the antigen is RNA. The methods and agents described herein are, in particular, useful for the treatment of diseases characterized by diseased cells expressing an antigen the antigen receptor or antigen receptor-engineered immune effector cells are directed to.

### BACKGROUND

The immune system plays an important role in cancer, autoimmunity, allergy as well as in pathogen-associated diseases. T cells and NK cells are important mediators of anti-tumor immune responses. CD8⁺ T cells and NK cells can directly lyse tumor cells. CD4⁺ T cells, on the other hand, can mediate the influx of different immune subsets including CD8⁺ T cells and NK cells into the tumor. CD4⁺ T cells are able to license dendritic cells (DCs) for the priming of anti-tumor CD8⁺ T cell responses and can act directly on tumor cells via IFNγ mediated MHC upregulation and growth inhibition. CD8⁺ as well as CD4⁺ tumor specific T-cell responses can be induced via vaccination or by adoptive transfer of T cells.

Adoptive cell transfer (ACT) based immunotherapy can be broadly defined as a form of passive immunization with previously sensitized T cells that are transferred to non-immune recipients or to the autologous host after ex vivo expansion from low precursor frequencies to clinically relevant cell numbers. Cell types that have been used for ACT experiments are lymphokine-activated killer (LAK) cells (Mule, J. J. et al. (1984) Science 225, 1487-1489; Rosenberg, S. A. et al. (1985) N. Engl. J. Med. 313, 1485-1492), tumor-infiltrating lymphocytes (TILs) (Rosenberg, S. A. et al. (1994) J. Natl. Cancer Inst. 86, 1159-1166), donor lymphocytes after hematopoietic stem cell transplantation (HSCT) as well as tumor-specific T cell lines or clones (Dudley, M. E. et al. (2001) J. Immunother. 24, 363-373; Yee, C. et al. (2002) Proc. Natl. Acad. Sci. U.S.A. 99, 16168-16173). An alternative approach is the adoptive transfer of autologous T cells reprogrammed to express a tumor-reactive immunoreceptor of defined specificity during short-time ex vivo culture followed by reinfusion into the patient (Kershaw M. H. et al. (2013) Nature Reviews Cancer 13 (8):525-41). This strategy makes ACT applicable to a variety of common malignancies even if tumor-reactive T cells are absent in the patient. For example, adoptive transfer of chimeric antigen receptor modified T cells (CART cells) is investigated in an extensive number of clinical trials worldwide. Chimeric antigen receptors (CARs) are a type of antigen-targeted receptor composed of intracellular T cell signaling domains fused to extracellular antigen-binding moieties, most commonly single-chain variable fragments (scFvs) from monoclonal antibodies. CARs directly recognize cell surface antigens, independent of MHC-mediated presentation, permitting the use of a single receptor construct specific for any given antigen in all patients. In general, CARs fuse antigen-recognition domains to the CD3ζ activation chain of the T cell receptor (TCR) complex and comprise secondary costimulatory signals in tandem with CD3, including intracellular domains from CD28 or a variety of TNF receptor family molecules such as 4-1BB (CD137) and OX40 (CD134). CARs dramatically improved antitumor efficacy, showing remarkable clinical efficacy especially in patients suffering from hematological malignancies (Hartmann, J. et al. EMBO Mol. Med. 9, 1183-1197 (2017)). Recently, two CAR T-cell therapies have received approval for the treatment of B-cell acute lymphoblastic leukaemia (Kymriah®) and diffuse large B-cell lymphoma (Yescarta®) by the FDA and EMA (Zheng, P. et al. Drug. Discov. Today 6, 1175-1182 (2018)). For solid tumors adoptive transfer of T cells, however, has shown limited efficacy so far and requires improvement (Newick, K. et al. Annu. Rev. Med. 68, 139-152 (2017)).

Key challenges for the application of antigen receptor-engineered immune cells in solid cancers are, in particular, the non-persistence of transferred cells. Furthermore, the generation of large amounts of cells for adoptive cell transfer still remains a challenge and the number of cells which can be administered to a patient for adoptive cell transfer is generally limited. In addition, approaches for the transfer of large amounts of engineered T cells into a host pose the risk of severe adverse events. Therefore, it would be desirable to provide a limited amount of engineered immune effector cells such as T cells to a patient that can be expanded in the patient after they have proven to be safe.

Here we introduce a novel concept to overcome inefficient CAR T cell stimulation in vivo which is generally the case in solid cancer patients. We demonstrate that a nanoparticulate RNA vaccine designed for body-wide delivery of the CAR antigen into lymphoid compartments stimulates adoptively transferred CAR T cells. Presentation of the natively folded target on resident dendritic cells promotes robust cognate and selective expansion of CAR T cells. Consequently, improved engraftment of CAR T cells and regression of large tumors in difficult-to-treat mouse models is accomplished at sub-therapeutic CAR T cell doses. The vaccine approach described herein is expected to be particularly suitable in connection with therapies involving in vivo generation of antigen receptor-engineered immune effector cells which is expected to produce only small amounts of cells.

The methods provided herein allow to only provide small amounts of antigen receptor-engineered immune effector cells such as T cells to a patient, e.g., by in vivo generation of antigen receptor-engineered immune effector cells, and then expand the cells in vivo to result in therapeutic amounts. It is demonstrated herein that the approach described herein is even effective in the treatment of solid tumors or cancers.

### SUMMARY

The present invention generally embraces the treatment of diseases by targeting cells such as diseased cells expressing an antigen such as a tumor antigen. The cells may express the antigen on the cell surface for recognition by a chimeric antigen receptor (CAR) or in the context of MHC for recognition by a T cell receptor (TCR). The methods provide for the selective eradication of such cells expressing an antigen, thereby minimizing adverse effects to normal cells not expressing the antigen. Immune effector cells genetically modified to express a chimeric antigen receptor (CAR) or a T cell receptor (TCR) targeting the cells through binding to the antigen (or a procession product thereof) are provided to a subject such as by administration of genetically modified immune effector cells to the subject or generation of genetically modified immune effector cells in the subject. A vaccine antigen (which may be the disease-associated antigen or a variant thereof (e.g. a peptide or protein comprising an epitope of the disease-associated antigen), nucleic acid coding therefor, or cells expressing the antigen are administered to provide (optionally following expression of the nucleic acid by appropriate target cells) antigen for immune effector cell stimulation, priming and/or expansion. Immune effector cells stimulated, primed and/or expanded in the patient are able to recognize and eradicate diseased cells expressing an antigen. In one embodiment, the immune effector cells are T cells. In one embodiment, the immune effector cells are directed against a tumor or cancer. In one embodiment, the target cell population or target tissue is tumor cells or tumor tissue, in particular of a solid tumor. In one embodiment, the target antigen is a tumor antigen.

The methods and agents described herein are, in particular, useful for the treatment of diseases characterized by diseased cells expressing an antigen the immune effector cells are directed to. In one embodiment, the immune effector cells by means of a chimeric antigen receptor (CAR) have a binding specificity for vaccine antigen and disease-associated antigen when present on antigen presenting cells and diseased cells, respectively. In one embodiment, the immune effector cells by means of a T cell receptor (TCR) having a binding specificity for a procession product of vaccine antigen and disease-associated antigen when presented on antigen presenting cells and diseased cells, respectively. CARs are molecules that combine specificity for a desired antigen (e.g., tumor antigen) which preferably is antibody-based with a T cell receptor-activating intracellular domain to generate a chimeric protein that exhibits a specific cellular immune activity (e.g., a specific anti-tumor cellular immune activity). Preferably, a cell can be genetically modified to stably express an antigen receptor on its surface, conferring novel antigen specificity that may be MHC independent. In one embodiment, immune effector cells either from a subject to be treated or from a different subject are administered to the subject to be treated. The administered immune effector cells may be genetically modified ex vivo prior to administration or genetically modified in vivo in the subject following administration to express an antigen receptor described herein. In one embodiment, the immune effector cells are endogenous in a subject to be treated (thus, are not administered to the subject to be treated) and are genetically modified in vivo in the subject to express an antigen receptor described herein. Accordingly, immune effector cells may be genetically modified, ex vivo or in vivo, to express an antigen receptor. Thus, such genetic modification with antigen receptor may be effected in vitro and subsequently the immune effector cells administered to a subject in need of treatment or may be effected in vivo in a subject in need of treatment. Thus, in one aspect, the present invention generally embraces the treatment of diseases by targeting cells expressing an antigen such as diseased cells, in particular cancer cells expressing a tumor antigen. The target cells may express the antigen on the cell surface or may present a procession product of the antigen. In one embodiment, the antigen is a tumor-associated antigen and the disease is cancer. Such treatment provides for the selective eradication of cells that express an antigen, thereby minimizing adverse effects to normal cells not expressing the antigen. In one embodiment, vaccine antigen, polynucleotide coding therefor or cells expressing vaccine antigen are administered to provide (optionally following expression of the polynucleotide by appropriate target cells) antigen for stimulation, priming and/or expansion of immune effector cells genetically modified to express an antigen receptor, wherein the immune effector cells are targeted to the antigen or a procession product thereof and the immune response is an immune response to a target cell population or target tissue expressing the antigen. In one embodiment, the polynucleotide encoding the vaccine antigen is RNA. Immune effector cells such as T cells stimulated, primed and/or expanded in the patient are able to recognize cells expressing an antigen resulting in the eradication of diseased cells. In one embodiment, vaccine antigen-encoding RNA is targeted to secondary lymphoid organs.

The invention in one aspect relates to a method for treating a subject comprising:

(i) providing sub-therapeutic amounts of immune effector cells genetically modified to express an antigen receptor to the subject, and

(ii) administering to the subject an antigen targeted by the antigen receptor, a polynucleotide encoding the antigen, or a host cell genetically modified to express the antigen.

In one embodiment, the method is a method of inducing an immune response in said subject. In one embodiment, the immune response is a T cell-mediated immune response. In one embodiment, the immune response is an immune response to a target cell population or target tissue expressing an antigen. In one embodiment, the target cell population or target tissue is cancer cells or cancer tissue. In one embodiment, the cancer cells or cancer tissue is solid cancer.

The invention in another aspect relates to a method for treating a subject having a disease, disorder or condition associated with expression or elevated expression of an antigen comprising:

(i) providing sub-therapeutic amounts of immune effector cells genetically modified to express an antigen receptor, the antigen receptor being targeted to the antigen associated with the disease, disorder or condition or cells expressing the antigen associated with the disease, disorder or condition, to the subject, and

(ii) administering to the subject an antigen targeted by the antigen receptor, a polynucleotide encoding the antigen, or a host cell genetically modified to express the antigen.

In one embodiment, the disease, disorder or condition is cancer and the antigen associated with the disease, disorder or condition is a tumor antigen. In one embodiment, the disease, disorder or condition is solid cancer.

The invention in another aspect relates to a method for treating a subject having a solid cancer associated with expression or elevated expression of a tumor antigen comprising:

(i) providing immune effector cells genetically modified to express an antigen receptor, the antigen receptor being targeted to the tumor antigen or cells expressing the tumor antigen, to the subject, and

(ii) administering to the subject an antigen targeted by the antigen receptor, a polynucleotide encoding the antigen, or a host cell genetically modified to express the antigen.

In one embodiment, the immune effector cells genetically modified to express an antigen receptor are provided to the subject in sub-therapeutic amounts.

In one embodiment of all aspects disclosed herein, the immune effector cells genetically modified to express an antigen receptor are provided to the subject by administering the immune effector cells genetically modified to express an antigen receptor or by generating the immune effector cells genetically modified to express an antigen receptor in the subject.

The invention in another aspect relates to a method for treating a subject comprising:

(i) generating immune effector cells genetically modified to express an antigen receptor in the subject, and

(ii) administering to the subject an antigen targeted by the antigen receptor, a polynucleotide encoding the antigen, or a host cell genetically modified to express the antigen.

In one embodiment, the method is a method of inducing an immune response in said subject. In one embodiment, the immune response is a T cell-mediated immune response. In one embodiment, the immune response is an immune response to a target cell population or target tissue expressing an antigen. In one embodiment, the target cell population or target tissue is cancer cells or cancer tissue. In one embodiment, the cancer cells or cancer tissue is solid cancer.

The invention in another aspect relates to a method for treating a subject having a disease, disorder or condition associated with expression or elevated expression of an antigen comprising:

(i) generating immune effector cells genetically modified to express an antigen receptor, the antigen receptor being targeted to the antigen associated with the disease, disorder or condition or cells expressing the antigen associated with the disease, disorder or condition, in the subject, and

(ii) administering to the subject an antigen targeted by the antigen receptor, a polynucleotide encoding the antigen, or a host cell genetically modified to express the antigen.

In one embodiment, the disease, disorder or condition is cancer and the antigen associated with the disease, disorder or condition is a tumor antigen. In one embodiment, the disease, disorder or condition is solid cancer.

The invention in another aspect relates to a method for treating a subject having a solid cancer associated with expression or elevated expression of a tumor antigen comprising:

(i) generating immune effector cells genetically modified to express an antigen receptor, the antigen receptor being targeted to the tumor antigen or cells expressing the tumor antigen, in the subject, and

(ii) administering to the subject an antigen targeted by the antigen receptor, a polynucleotide encoding the antigen, or a host cell genetically modified to express the antigen.

In one embodiment of all aspects disclosed herein, the immune effector cells genetically modified to express an antigen receptor are generated in the subject in sub-therapeutic amounts.

In one embodiment of all aspects disclosed herein, the method is a method for treating or preventing cancer in a subject. In one embodiment, the cancer is solid cancer. In one embodiment, the cancer is associated with expression or elevated expression of a tumor antigen targeted by the antigen receptor.

In one embodiment of all aspects disclosed herein, the antigen receptor is a chimeric antigen receptor (CAR) or T cell receptor (TCR).

In one embodiment of all aspects disclosed herein, the polynucleotide encoding the antigen is RNA.

In one embodiment of all aspects disclosed herein, the polynucleotide encoding the antigen is present in the form of particles further comprising a delivery vehicle, in particular lipoplex particles.

In one embodiment of all aspects disclosed herein, the host cell genetically modified to express the antigen comprises a polynucleotide encoding the antigen.

In one embodiment of all aspects disclosed herein, the immune effector cells genetically modified to express an antigen receptor comprise a polynucleotide encoding the antigen receptor.

In one embodiment of all aspects disclosed herein, the immune effector cells are T cells.

The invention in another aspect relates to a kit comprising:

(i) immune effector cells genetically modified to express an antigen receptor or a polynucleotide encoding an antigen receptor, and

(ii) an antigen targeted by the antigen receptor, a polynucleotide encoding the antigen, or a host cell genetically modified to express the antigen.

In one embodiment, the polynucleotide encoding an antigen receptor is useful for in vivo genetic modification of immune effector cells to express an antigen receptor.

In one embodiment, the kit further comprises instructional material for use of the kit in any one of the methods described herein.

In a further aspect, the invention relates to the agents and compositions described herein, e.g., immune effector cells genetically modified to express an antigen receptor, and/or antigen, polynucleotide encoding an antigen, or host cell genetically modified to express an antigen, for therapeutic use, in particular for use in the methods described herein.

Other features and advantages of the instant invention will be apparent from the following detailed description and claims.

### BRIEF DESCRIPTION OF THE DRAWINGS

FIG. 1. The oncofetal antigen CLDN6 is an ideal target for CAR T-cell therapy. (A, B) Expression of CLDN6 transcript and protein in human tissues as analyzed by (A) qRT-PCR and (B) IHC. (a) adrenal gland, (b) fallopian tube, (c) kidney, (d) liver, (e) thyroid, (f) prostate, (g) esophagus, (h) stomach, (i) colon, (j) cerebrum, (k) cerebellum, (l) spinal cord, (m) thymus, (n) spleen, (o) bone marrow, (p) pancreas, (q) skin, (r) bladder, (s) placenta, (t) heart muscle, (u) striated muscle, (v) testis, (w) ovary, (x) lung, (CA1) testicular cancer, (CA2) ovarian cancer and (CA3) lung cancer; (C) Design of CLDN6-CAR. (D) Dependency of lysis by human CLDN6-CAR T cells (E:T=20:1, mean+/−SD of technical triplicates, right) on the level of CLDN6 surface expression. Colo-699-N cells (no endogenous claudin expression) electroporated with escalating amounts of CLDN6-RNA as analyzed by flow cytometry (left). (E) Surface expression of highly homologous claudins on Colo-699-N cells electroporated with CLDN-RNAs assessed by flow cytometry (control: isotype antibody, left) and analysis of cross-recognition and lysis by co-cultured CLDN6-CAR T cells (E:T=7:1, mean+/−SD of technical triplicates, right). (F) Human tumor cell lines analyzed by flow cytometry for CLDN6 and CLDN9 surface expression (upper panel) were co-cultured with CLDN6-CAR or non-transduced T cells (E:T=10:1). IFNγ secretion (middle; mean+SD of technical duplicates) and expression of activation markers OX40 on CD4⁺ and 4-1BB on CD8⁺ T cells after co-culture (lower panel) as assessed by flow cytometry. (G) Serial killing of CLDN6^{pos} and CLDN6^{-/-} PA-1 tumor spheroids co-cultured with either CLDN6-CAR or non-transduced T cells (E:T=10:1) as measured by GFP real-time imaging (mean of technical triplicates). (H) NSG mice bearing subcutaneous CLDN6^{pos} OV90 xenografts were treated with human T cells transduced with CLDN6-CAR or GFP. Tumor and T-cell characteristics (left) and tumor growth kinetics in individual mice (right) were analyzed. ACT; adoptive cell transfer.

FIG. 2. Activation of CAR T cells by RNA-LPX-mediated display of the CAR target on dendritic cells is strictly antigen-specific and dose-dependent. (A) Surface expression of CLDN6 (upper panel) and CLDN18.2 (lower panel) on DCs pulsed with RNA-LPX encoding the respective CLDN assessed by flow cytometry. (B) Cytokine secretion of CAR T cells analyzed by a multiplex assay after 24 h of co-culture of claudin-expressing DCs with CFSE-labeled CLDN6-CAR (upper panel) or CLDN18.2-CAR T cells (lower panel). Proliferation of CD4⁺ and CD8⁺ CART cells was analyzed by flow cytometry after 5 days (right). Mean+SD of technical triplicates are indicated; nd=not detected. (C) Surface expression of CLDN6 on splenic immune cell populations of BALB/c mice analyzed by flow cytometry 24 h after a single i.v. injection of 25 μg RNA-LPX encoding either CLDN6 or an irrelevant control (Mean+SEM of biological duplicates). (D) CAR T-cell proliferation in secondary lymphoid tissues resected 48 h after i.v. administration of RNA-LPX (CLDN18.2 as control). mean+/−SEM of biological replicates (n=5 per group). LN, lymph node. P-values were determined by unpaired Student's t-test. *P<0.05, **P<0.01, ***P<0.001, ****P<0.0001.

FIG. 3. RNA-LPX vaccination mediates efficient in vivo expansion, superior functionality and memory formation of CAR T cells. (A-B) Impact of dose level of i.v. administered target-antigen encoding RNA-LPX on expansion of CART cells in vivo. Luciferase (Luc)-expressing Thy1.1⁺ CLDN6-CAR T cells (10⁶/animal) were transferred into lymphodepleted Thy1.2⁺ C57BL/6-albino mice (n=5/group) and 8 days later mice were injected i.v. in total 40 μg RNA-LPX but titrated the amount of CLDN6 as indicated. (A, left) Kinetics of CAR T-cell expansion by bioluminescence imaging (BLI) and (A, right) the expansion index of CAR T cells and (B) frequencies of KLRG1- and CD62L-expressing endogenous (Thy1.2⁺) and transferred (Thy1.1⁺) CD8⁺ T cells in peripheral blood day 11 post ACT by flow cytometry (mean±SEM). (C) Impact of repetitive i.v. dosing of target-antigen encoding RNA-LPX on expansion of CAR T cells in vivo. BLI kinetics of different dose levels of Thy1.1⁺ Luc-expressing CLDN6-CAR T cells transferred into lymphodepleted Thy1.2⁺ C57BL/6-albino mice. Mice in the lowest CAR T-cell dose group (10³) were vaccinated twice with 20 μg CLDN6 RNA-LPX (n=6), while all other groups received saline (n=4/group). Representative imaging (left) and mean+/−SEM of treatment groups (middle). Thy1.1⁺ population in peripheral blood of individual mice determined by flow cytometry (right). (D) Ex vivo cytotoxic activity of low dose CAR T cells from CLDN6-LPX vaccinated mice (1.5×10⁶ CAR-T+CLDN6-LPX) compared to high-dose CAR T cells sorted from control-vaccinated mice (7.5×10⁶ CAR-T+CLDN18.2-LPX) three (time point a) and seven (time point b) days after second vaccination (n=5/treatment group/time point). Sorted, but pooled CAR T cells/treatment group were 20 h co-cultured in the presence with human CLDN6-transduced B16 mouse melanoma cells or WT control at indicated E:T ratios (mean±SD of technical triplicates). (E) Luc/GFP-expressing Thy1.1⁺ CLDN6-CAR T cells transferred into lymphodepleted Thy1.2⁺ C57BL/6-albino mice (n=2-3/group) followed by repetitive vaccination with RNA-LPX (Oval as control). CAR T-cell kinetics by BLI (left, mean±SEM of treatment groups). Frequency of GFP⁺ CAR T-cell population in peripheral blood in pre-treatment samples (time point a, day 7 post ACT) and after 3^{rd} RNA-LPX treatment (time point b, day 26 post ACT) (middle). Frequency of memory CAR T cells in the CD8⁺ T cell population 31 days after 4th treatment (right; time point c; day 80 after ACT). P-values determined by paired (C) and unpaired two-tailed Student's t-test (D, E). *P<0.05, **P<0.01, ***P<0.001, **** P<0.0001.

FIG. 4. Sub-therapeutic CAR T-cell doses are rendered anti-tumorally efficacious against large tumors by RNA-LPX vaccination. (A, B) Mice with large established tumors were lymphodepleted, treated with syngeneic non-transduced (non-transd.) or CLDN-CAR redirected mouse T cells followed by single i.v. administration of CLDN or control RNA-LPX. Tumor growth (mean±SEM, left) and survival (right) were determined. (A) CLDN6-CAR was tested in C57BL/6 mice tumor-bearing LL/2-LLc1 tumors transduced with human CLDN6 (n=9-10/group; tumor size at start of treatment 209 mm³) and (B) CLDN18.2-CAR in BALB/c mice bearing mouse CLDN18.2-transduced CT26 (n=9/group; tumor size at start of treatment 77 mm³). (C) Human Luc-expressing CLDN-specific CAR T cells in naïve NSG mice vaccinated twice with CLDN-LPX. CAR T-cell expansion was analyzed by measuring the splenic BLI signal (mean + or − SEM of 2-3 mice/group) (D) NSG mice with OV90 xenograft tumors (tumor size at start of treatment 60 mm³) were treated with a sub-therapeutic dose of human CLDN6-CAR (10⁵/animal) or non-transduced T cells followed by 3 weekly repetitions of RNA-LPX coding for CLDN6 or a control. Tumor growth curves (mean±SEM of 9-10 mice/group, left) and representative CAR T-cell frequencies after 3^{rd} RNA-LPX treatment in peripheral blood as assessed by flow cytometry (right). (E) Maintaining frequency of circulating CART cells within a therapeutic window by CARVac. P-values were determined by two-way ANOVA with Tukey's multiple-comparisons test (A left, B left, D left). Time points from ACT until the end of at least the control group were considered in the calculation. Survival benefit was determined with the log-rank test (A right, B right). ns=not significant, *P<0.05, **P<0.01, ***P<0.001, **** P<0.0001.

FIG. 5: Repetitive treatment of antigen-RNA-LPX leads to in vivo expansion of OT1-TCR modified T cells

2.5 Gy irradiated (XRAD320) C57BL/6BrdCrHsd-Tyr^{c} mice (n=2-3/group) were i.v. engrafted with 5×10⁶ OT1-TCR-Luc-GFP transduced C57Bl/6-Thy1.1⁺ T cells. 8 days after ACT, mice received Oval or hCLDN6 (ctrl RNA) encoding mRNA lipoplex vaccination (RNA-LPX; 20 μg, i.v.) and subsequent IL-2/7 support (1 μg/cytokine mRNA/mouse, i.p.). The treatment was repeated on day 15 and 22 after ACT. Sequential bioluminescence imaging and peripheral blood analysis were performed to monitor expansion and enrichment of transferred T cells on day 7 (baseline) up to day 25 after ACT. A) Quantification of in vivo bioluminescence during and after the expansion rounds with Oval-RNA-LPX or control RNA-LPX in the presence of indicated nucleoside-modified-formulated cytokine RNAs are shown (mean + or − s.e.m.). Dotted, vertical lines indicate the time point of RNA-LPX/cytokine treatment. B) Flow cytometry analysis of transferred Thy1.1+ T cells were performed in peripheral blood of one exemplary mouse/treatment group on day 7 (baseline), day 11 (3 days post 1^{st} vacc.), day 18 (3 days post 2^{nd} vacc.) and day 25 (3 days post 3^{rd} vacc.) after ACT. Numbers in histograms indicate the mean fluorescence intensity (MFI) of GFP expressing Thy1.1+ T cells. GFP has been used as surrogate marker for OT1-TCR transduced T cells. LPX: lipoplex Luc: effective firefly luciferase

### DETAILED DESCRIPTION

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present disclosure will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present disclosure will be described. These elements are listed with specific embodiments, however, it should be understood that they may be combined in any manner and in any number to create additional embodiments. The variously described examples and embodiments should not be construed to limit the present disclosure to only the explicitly described embodiments. This description should be understood to disclose and encompass embodiments which combine the explicitly described embodiments with any number of the disclosed elements. Furthermore, any permutations and combinations of all described elements should be considered disclosed by this description unless the context indicates otherwise.

The term “about” means approximately or nearly, and in the context of a numerical value or range set forth herein in one embodiment means ±20%, ±10%, ±5%, or ±3% of the numerical value or range recited or claimed.

The terms “a” and “an” and “the” and similar reference used in the context of describing the disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the disclosure and does not pose a limitation on the scope of the claims. No language in the specification should be construed as indicating any non-claimed element essential to the practice of the disclosure.

Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of”.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

In the following, definitions will be provided which apply to all aspects of the present disclosure. The following terms have the following meanings unless otherwise indicated. Any undefined terms have their art recognized meanings.

### Definitions

Terms such as “reduce”, “decrease”, “inhibit” or “impair” as used herein relate to an overall decrease or the ability to cause an overall decrease, preferably of 5% or greater, 10% or greater, 20% or greater, more preferably of 50% or greater, and most preferably of 75% or greater, in the level, e.g. in the level of binding.

Terms such as “increase”, “enhance” or “exceed” preferably relate to an increase or enhancement by about at least 10%, preferably at least 20%, preferably at least 30%, more preferably at least 40%, more preferably at least 50%, even more preferably at least 80%, and most preferably at least 100%, at least 200%, at least 500%, or even more.

According to the disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” or “polypeptide” refers to large peptides, in particular peptides having at least about 151 amino acids, but the terms “peptide”, “protein” and “polypeptide” are used herein usually as synonyms.

A “therapeutic protein” has a positive or advantageous effect on a condition or disease state of a subject when provided to the subject in a therapeutically effective amount. In one embodiment, a therapeutic protein has curative or palliative properties and may be administered to ameliorate, relieve, alleviate, reverse, delay onset of or lessen the severity of one or more symptoms of a disease or disorder. A therapeutic protein may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Examples of therapeutically active proteins include, but are not limited to, antigens for vaccination and cytokines.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable e.g. by translation of a truncated open reading frame that lacks the 3′-end of the open reading frame. A fragment shortened at the N-terminus (C-terminal fragment) is obtainable e.g. by translation of a truncated open reading frame that lacks the 5′-end of the open reading frame, as long as the truncated open reading frame comprises a start codon that serves to initiate translation. A fragment of an amino acid sequence comprises e.g. at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence.

By “variant” or “variant protein” or “variant polypeptide” herein is meant a protein that differs from a wild type protein by virtue of at least one amino acid modification. The parent polypeptide may be a naturally occurring or wild type (WT) polypeptide, or may be a modified version of a wild type polypeptide. Preferably, the variant polypeptide has at least one amino acid modification compared to the parent polypeptide, e.g. from 1 to about 20 amino acid modifications, and preferably from 1 to about 10 or from 1 to about 5 amino acid modifications compared to the parent.

By “parent polypeptide”, “parent protein”, “precursor polypeptide”, or “precursor protein” as used herein is meant an unmodified polypeptide that is subsequently modified to generate a variant. A parent polypeptide may be a wild type polypeptide, or a variant or engineered version of a wild type polypeptide.

By “wild type” or “WT” or “native” herein is meant an amino acid sequence that is found in nature, including allelic variations. A wild type protein or polypeptide has an amino acid sequence that has not been intentionally modified.

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all splice variants, posttranslationally modified variants, conformations, isoforms and species homologs, in particular those which are naturally expressed by cells. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In one embodiment, conservative amino acid substitutions include substitutions within the following groups:

glycine, alanine;

valine, isoleucine, leucine;

aspartic acid, glutamic acid;

asparagine, glutamine;

serine, threonine;

lysine, arginine; and

phenylalanine, tyrosine.

Preferably the degree of similarity, preferably identity between a given amino acid sequence and an amino acid sequence which is a variant of said given amino acid sequence will be at least about 60%, 65%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, preferably continuous amino acids. In preferred embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences.

The term “percentage identity” is intended to denote a percentage of amino acid residues which are identical between the two sequences to be compared, obtained after the best alignment, this percentage being purely statistical and the differences between the two sequences being distributed randomly and over their entire length. Sequence comparisons between two amino acid sequences are conventionally carried out by comparing these sequences after having aligned them optimally, said comparison being carried out by segment or by “window of comparison” in order to identify and compare local regions of sequence similarity. The optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The percentage identity is calculated by determining the number of identical positions between the two sequences being compared, dividing this number by the number of positions compared and multiplying the result obtained by 100 so as to obtain the percentage identity between these two sequences.

Homologous amino acid sequences exhibit according to the disclosure at least 40%, in particular at least 50%, at least 60%, at least 70%, at least 80%, at least 90% and preferably at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or proteins having substitutions, additions, insertions or deletions, is described in detail in Sambrook et al. (1989), for example. Furthermore, the peptides and amino acid variants described herein may be readily prepared with the aid of known peptide synthesis techniques such as, for example, by solid phase synthesis and similar methods.

In one embodiment, a fragment or variant of an amino acid sequence (peptide or protein) is preferably a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to antigens, one particular function is one or more immunostimulating activities displayed by the amino acid sequence from which the fragment or variant is derived and/or binding to the receptor(s) the amino acid sequence from which the fragment or variant is derived binds to. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., binding to a target molecule. In one embodiment, the modifications in the amino acid sequence of the parent molecule or sequence do not significantly affect or alter the binding characteristics of the molecule or sequence. In different embodiments, binding of the functional fragment or functional variant may be reduced but still significantly present, e.g., binding of the functional variant may be at least 50%, at least 60%, at least 70%, at least 80%, or at least 90% of the parent molecule or sequence. However, in other embodiments, binding of the functional fragment or functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. Preferably, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

As used herein, an “instructional material” or “instructions” includes a publication, a recording, a diagram, or any other medium of expression which can be used to communicate the usefulness of the compositions and methods of the invention. The instructional material of the kit of the invention may, for example, be affixed to a container which contains the compositions of the invention or be shipped together with a container which contains the compositions. Alternatively, the instructional material may be shipped separately from the container with the intention that the instructional material and the compositions be used cooperatively by the recipient.

“Isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell.

The term “recombinant” in the context of the present invention means “made through genetic engineering”. Preferably, a “recombinant object” such as a recombinant cell in the context of the present invention is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “specifically binds”, as used herein, is meant a molecule such as an antibody or CAR which recognizes a specific antigen, but does not substantially recognize or bind other molecules in a sample or in a subject. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding”, can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “genetic modification” includes the transfection of cells with nucleic acid. The term “transfection” relates to the introduction of nucleic acids, in particular RNA, into a cell. For purposes of the present invention, the term “transfection” also includes the introduction of a nucleic acid into a cell or the uptake of a nucleic acid by such cell, wherein the cell may be present in a subject, e.g., a patient. Thus, according to the present invention, a cell for transfection of a nucleic acid described herein can be present in vitro or in vivo, e.g. the cell can form part of an organ, a tissue and/or an organism of a patient. According to the invention, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection. Generally, cells that are genetically modified to express an antigen receptor are stably transfected with nucleic acid encoding the antigen receptor, while, generally, nucleic acid encoding antigen is transiently transfected into cells.

Immune Effector Cells

The cells used in connection with the present invention and into which nucleic acids (DNA or RNA) encoding antigen receptors may be introduced include, in particular, immune effector cells such as cells with lytic potential, in particular lymphoid cells, and are preferably T cells, in particular cytotoxic lymphocytes, preferably selected from cytotoxic T cells, natural killer (NK) cells, and lymphokine-activated killer (LAK) cells. Upon activation, each of these cytotoxic lymphocytes triggers the destruction of target cells. For example, cytotoxic T cells trigger the destruction of target cells by either or both of the following means. First, upon activation T cells release cytotoxins such as perforin, granzymes, and granulysin. Perforin and granulysin create pores in the target cell, and granzymes enter the cell and trigger a caspase cascade in the cytoplasm that induces apoptosis (programmed cell death) of the cell. Second, apoptosis can be induced via Fas-Fas ligand interaction between the T cells and target cells. The cells used in connection with the present invention will preferably be autologous cells, although heterologous cells or allogenic cells can be used.

The term “effector functions” in the context of the present invention includes any functions mediated by components of the immune system that result, for example, in the killing of diseased cells such as tumor cells, or in the inhibition of tumor growth and/or inhibition of tumor development, including inhibition of tumor dissemination and metastasis. Preferably, the effector functions in the context of the present invention are T cell mediated effector functions. Such functions comprise in the case of a helper T cell (CD4⁺ T cell) the release of cytokines and/or the activation of CD8⁺ lymphocytes (CTLs) and/or B cells, and in the case of CTL the elimination of cells, i.e., cells characterized by expression of an antigen, for example, via apoptosis or perforin-mediated cell lysis, production of cytokines such as IFN-γ and TNF-α, and specific cytolytic killing of antigen expressing target cells.

The term “immune effector cell” or “immunoreactive cell” in the context of the present invention relates to a cell which exerts effector functions during an immune reaction. An “immune effector cell” in one embodiment is capable of binding an antigen such as an antigen presented in the context of MHC on a cell or expressed on the surface of a cell and mediating an immune response. For example, immune effector cells comprise T cells (cytotoxic T cells, helper T cells, tumor infiltrating T cells), B cells, natural killer cells, neutrophils, macrophages, and dendritic cells. Preferably, in the context of the present invention, “immune effector cells” are T cells, preferably CD4⁺ and/or CD8⁺ T cells. According to the invention, the term “immune effector cell” also includes a cell which can mature into an immune cell (such as T cell, in particular T helper cell, or cytolytic T cell) with suitable stimulation. Immune effector cells comprise CD34⁺ hematopoietic stem cells, immature and mature T cells and immature and mature B cells. The differentiation of T cell precursors into a cytolytic T cell, when exposed to an antigen, is similar to clonal selection of the immune system.

Preferably, an “immune effector cell” recognizes an antigen with some degree of specificity, in particular if presented in the context of MHC or present on the surface of diseased cells such as cancer cells.

Preferably, said recognition enables the cell that recognizes an antigen to be responsive or reactive. If the cell is a helper T cell (CD4⁺ T cell) such responsiveness or reactivity may involve the release of cytokines and/or the activation of CD8⁺ lymphocytes (CTLs) and/or B cells. If the cell is a CTL such responsiveness or reactivity may involve the elimination of cells, i.e., cells characterized by expression of an antigen, for example, via apoptosis or perforin-mediated cell lysis. According to the invention, CTL responsiveness may include sustained calcium flux, cell division, production of cytokines such as IFN-γ and INF-α, up-regulation of activation markers such as CD44 and CD69, and specific cytolytic killing of antigen expressing target cells. CTL responsiveness may also be determined using an artificial reporter that accurately indicates CTL responsiveness. Such CTL that recognizes an antigen and are responsive or reactive are also termed “antigen-responsive CTL” herein.

In one embodiment, the immune effector cells are CAR-expressing immune effector cells. In one embodiment, the immune effector cells are TCR-expressing immune effector cells.

The immune effector cells to be used according to the invention may express an endogenous antigen receptor such as T cell receptor or B cell receptor or may lack expression of an endogenous antigen receptor.

A “lymphoid cell” is a cell which, optionally after suitable modification, e.g. after transfer of an antigen receptor such as a TCR or a CAR, is capable of producing an immune response such as a cellular immune response, or a precursor cell of such cell, and includes lymphocytes, preferably T lymphocytes, lymphoblasts, and plasma cells. A lymphoid cell may be an immune effector cell as described herein. A preferred lymphoid cell is a T cell which can be modified to express an antigen receptor on the cell surface. In one embodiment, the lymphoid cell lacks endogenous expression of a T cell receptor.

The terms “T cell” and “T lymphocyte” are used interchangeably herein and include T helper cells (CD4⁺ T cells) and cytotoxic T cells (CTLs, CD8⁺ T cells) which comprise cytolytic T cells. The term “antigen-specific T cell” or similar terms relate to a T cell which recognizes the antigen to which the T cell is targeted and preferably exerts effector functions of T cells. T cells are considered to be specific for antigen if the cells kill target cells expressing an antigen. T cell specificity may be evaluated using any of a variety of standard techniques, for example, within a chromium release assay or proliferation assay. Alternatively, synthesis of lymphokines (such as interferon-γ) can be measured.

T cells belong to a group of white blood cells known as lymphocytes, and play a central role in cell-mediated immunity. They can be distinguished from other lymphocyte types, such as B cells and natural killer cells by the presence of a special receptor on their cell surface called T cell receptors (TCR). The thymus is the principal organ responsible for the maturation of T cells. Several different subsets of T cells have been discovered, each with a distinct function.

T helper cells assist other white blood cells in immunologic processes, including maturation of B cells into plasma cells and activation of cytotoxic T cells and macrophages, among other functions. These cells are also known as CD4⁺ T cells because they express the CD4 glycoprotein on their surface. Helper T cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T cells destroy virally infected cells and tumor cells, and are also implicated in transplant rejection. These cells are also known as CD8⁺ T cells since they express the CD8 glycoprotein on their surface. These cells recognize their targets by binding to antigen associated with MHC class I, which is present on the surface of nearly every cell of the body.

“Regulatory T cells” or “Tregs” are a subpopulation of T cells that modulate the immune system, maintain tolerance to self-antigens, and prevent autoimmune disease. Tregs are immunosuppressive and generally suppress or downregulate induction and proliferation of effector T cells. Tregs express the biomarkers CD4, FoxP3, and CD25.

As used herein, the term “naïve T cell” refers to mature T cells that, unlike activated or memory T cells, have not encountered their cognate antigen within the periphery. Naïve T cells are commonly characterized by the surface expression of L-selectin (CD62L), the absence of the activation markers CD25, CD44 or CD69 and the absence of the memory CD45RO isoform.

As used herein, the term “memory T cells” refers to a subgroup or subpopulation of T cells that have previously encountered and responded to their cognate antigen. At a second encounter with the antigen, memory T cells can reproduce to mount a faster and stronger immune response than the first time the immune system responded to the antigen. Memory T cells may be either CD4⁺ or CD8⁺ and usually express CD45RO.

According to the invention, the term “T cell” also includes a cell which can mature into a T cell with suitable stimulation.

A majority of T cells have a T cell receptor (TCR) existing as a complex of several proteins. The actual T cell receptor is composed of two separate peptide chains, which are produced from the independent T cell receptor alpha and beta (TCRα and TCRβ) genes and are called α- and β-TCR chains. γδ T cells (gamma delta T cells) represent a small subset of T cells that possess a distinct T cell receptor (TCR) on their surface. However, in γδ T cells, the TCR is made up of one γ-chain and one δ-chain. This group of T cells is much less common (2% of total T cells) than the αβ T cells.

All T cells originate from hematopoietic stem cells in the bone marrow. Hematopoietic progenitors derived from hematopoietic stem cells populate the thymus and expand by cell division to generate a large population of immature thymocytes. The earliest thymocytes express neither CD4 nor CD8, and are therefore classed as double-negative (CD4⁻CD8⁻) cells. As they progress through their development they become double-positive thymocytes (CD4⁺CD8⁺), and finally mature to single-positive (CD4⁺CD8⁻ or CD4⁻ CD8⁺) thymocytes that are then released from the thymus to peripheral tissues.

T cells may generally be prepared in vitro or ex vivo, using standard procedures. For example, T cells may be isolated from bone marrow, peripheral blood or a fraction of bone marrow or peripheral blood of a mammal, such as a patient, using a commercially available cell separation system. Alternatively, T cells may be derived from related or unrelated humans, non-human animals, cell lines or cultures. A sample comprising T cells may, for example, be peripheral blood mononuclear cells (PBMC).

As used herein, the term “NK cell” or “Natural Killer cell” refers to a subset of peripheral blood lymphocytes defined by the expression of CD56 or CD16 and the absence of the T cell receptor. As provided herein, the NK cell can also be differentiated from a stem cell or progenitor cell.

Nucleic Acids

The term “polynucleotide” or “nucleic acid”, as used herein, is intended to include DNA and RNA such as genomic DNA, cDNA, mRNA, recombinantly produced and chemically synthesized molecules. A nucleic acid may be single-stranded or double-stranded. RNA includes in vitro transcribed RNA (IVT RNA) or synthetic RNA. According to the invention, a polynucleotide is preferably isolated.

Nucleic acids may be comprised in a vector. The term “vector” as used herein includes any vectors known to the skilled person including plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

In one embodiment of all aspects of the invention, nucleic acid such as nucleic acid encoding an antigen receptor or nucleic acid encoding a vaccine antigen is expressed in cells of the subject treated to provide the antigen receptor or vaccine antigen. In one embodiment of all aspects of the invention, the nucleic acid is transiently expressed in cells of the subject. Thus, in one embodiment, the nucleic acid is not integrated into the genome of the cells. In one embodiment of all aspects of the invention, the nucleic acid is RNA, preferably in vitro transcribed RNA. In one embodiment of all aspects of the invention, expression of the antigen receptor is at the cell surface. In one embodiment of all aspects of the invention, expression of the vaccine antigen is at the cell surface. In one embodiment of all aspects of the invention, the vaccine antigen is expressed and presented in the context of MHC.

In one embodiment of all aspects of the invention, the nucleic acid encoding the vaccine antigen is expressed in cells such as antigen presenting cells of the subject treated to provide the vaccine antigen for binding by the immune effector cells genetically modified to express an antigen receptor, said binding resulting in stimulation, priming and/or expansion of the immune effector cells genetically modified to express an antigen receptor.

The nucleic acids described herein may be recombinant and/or isolated molecules.

In the present disclosure, the term “RNA” relates to a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered RNAs are considered analogs of naturally-occurring RNA.

In certain embodiments of the present disclosure, the RNA is messenger RNA (mRNA) that relates to a RNA transcript which encodes a peptide or protein. As established in the art, mRNA generally contains a 5′ untranslated region (5′-UTR), a peptide coding region and a 3′ untranslated region (3′-UTR). In some embodiments, the RNA is produced by in vitro transcription or chemical synthesis. In one embodiment, the mRNA is produced by in vitro transcription using a DNA template where DNA refers to a nucleic acid that contains deoxyribonucleotides.

In one embodiment, RNA is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In one embodiment, the RNA may have modified ribonucleotides. Examples of modified ribonucleotides include, without limitation, 5-methylcytidine, pseudouridine and/or 1-methyl-pseudouridine.

In some embodiments, the RNA according to the present disclosure comprises a 5′-cap. In one embodiment, the RNA of the present disclosure does not have uncapped 5′-triphosphates. In one embodiment, the RNA may be modified by a 5′-cap analog. The term “5′-cap” refers to a structure found on the 5′-end of an mRNA molecule and generally consists of a guanosine nucleotide connected to the mRNA via a 5′ to 5′ triphosphate linkage. In one embodiment, this guanosine is methylated at the 7-position. Providing an RNA with a 5′-cap or 5′-cap analog may be achieved by in vitro transcription, in which the 5′-cap is co-transcriptionally expressed into the RNA strand, or may be attached to RNA post-transcriptionally using capping enzymes.

In some embodiments, the building block cap for RNA is m₂^{7,3′-O}Gppp(m₁^{2′-O})ApG (also sometimes referred to as m₂^{7,3′O}G(5′)ppp(5′)m^{2′-O}ApG), which has the following structure:

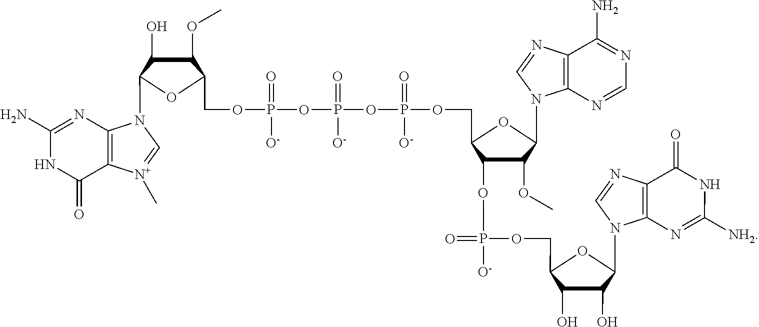

Below is an exemplary Cap1 RNA, which comprises RNA and m₂^{7,3′O}G(5′)ppp(5′)m^{2′-O}ApG:

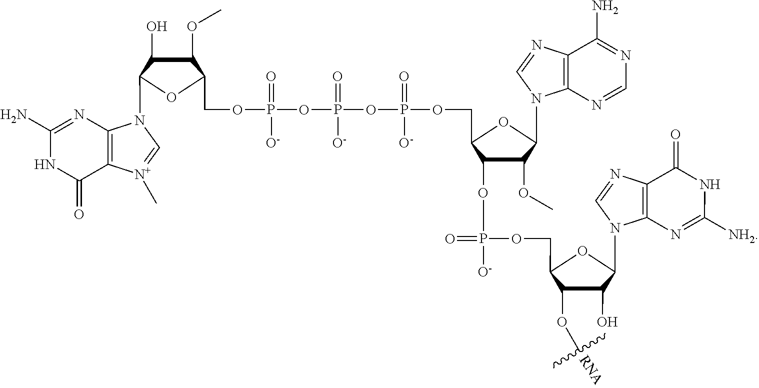

Below is another exemplary Cap1 RNA (no cap analog):

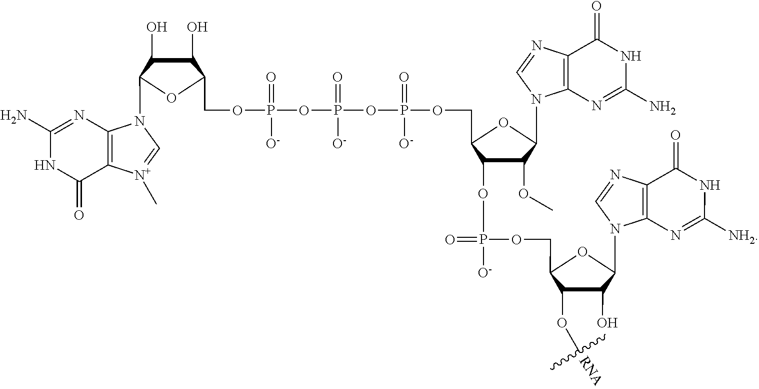

In some embodiments, the RNA is modified with “Cap0” structures using, in one embodiment, the cap analog anti-reverse cap (ARCA Cap (m₂^{7,3′O}G(5′)ppp(5′)G)) with the structure:

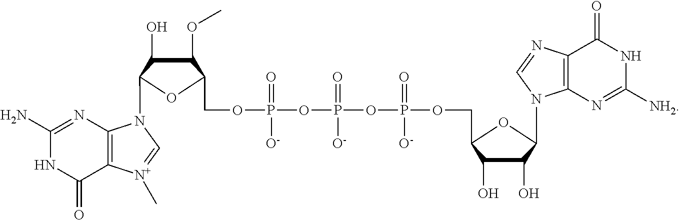

Below is an exemplary Cap0 RNA comprising RNA and m₂^{7,3′O}G(5′)ppp(5′)G:

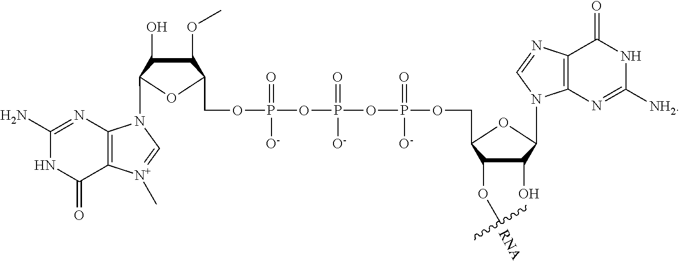

In some embodiments, the “Cap0” structures are generated using the cap analog Beta-S-ARCA (m₂^{7,2′O}G(5′)ppSp(5′)G) with the structure:

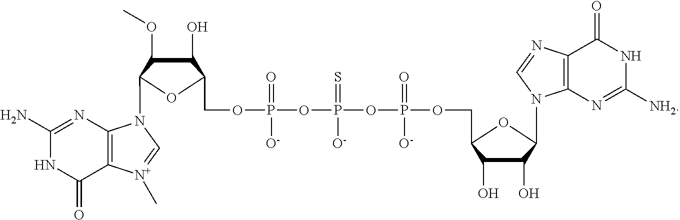

Below is an exemplary Cap0 RNA comprising Beta-S-ARCA (m₂^{7,2′O}G(5′)ppSp(5′)G) and RNA:

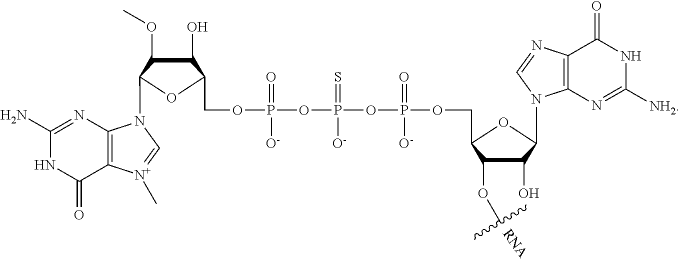

A particularly preferred Cap comprises the 5′-cap m₂^{7,2′O}G(5′)ppSp(5′)G.

In some embodiments, RNA according to the present disclosure comprises a 5′-UTR and/or a 3′-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′ end, upstream of the start codon of a protein-encoding region. A 5′-UTR is downstream of the 5′-cap (if present), e.g. directly adjacent to the 5′-cap. A 3′-UTR, if present, is located at the 3′ end, downstream of the termination codon of a protein-encoding region, but the term “3′-UTR” does preferably not include the poly(A) tail. Thus, the 3′-UTR is upstream of the poly(A) sequence (if present), e.g. directly adjacent to the poly(A) sequence.

In some embodiments, the RNA according to the present disclosure comprises a 3′-poly(A) sequence. As used herein, the term “poly-A tail” or “poly-A sequence” refers to an uninterrupted or interrupted sequence of adenylate residues which is typically located at the 3′-end of an RNA molecule. Poly-A tails or poly-A sequences are known to those of skill in the art and may follow the 3′-UTR in the RNAs described herein. An uninterrupted poly-A tail is characterized by consecutive adenylate residues. In nature, an uninterrupted poly-A tail is typical. RNAs disclosed herein can have a poly-A tail attached to the free 3′-end of the RNA by a template-independent RNA polymerase after transcription or a poly-A tail encoded by DNA and transcribed by a template-dependent RNA polymerase.

It has been demonstrated that a poly-A tail of about 120 A nucleotides has a beneficial influence on the levels of RNA in transfected eukaryotic cells, as well as on the levels of protein that is translated from an open reading frame that is present upstream (5′) of the poly-A tail (Holtkamp et al., 2006, Blood, vol. 108, pp. 4009-4017).

The poly-A tail may be of any length. In some embodiments, a poly-A tail comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly-A tail, typically at least 75%, at least 80%, at least 85%, at least 90%, at least 95%, at least 96%, at least 97%, at least 98%, or at least 99% by number of nucleotides in the poly-A tail are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly-A tail, i.e., 100% by number of nucleotides in the poly-A tail, are A nucleotides. The term “A nucleotide” or “A” refers to adenylate.

In some embodiments, a poly-A tail is attached during RNA transcription, e.g., during preparation of in vitro transcribed RNA, based on a DNA template comprising repeated dT nucleotides (deoxythymidylate) in the strand complementary to the coding strand. The DNA sequence encoding a poly-A tail (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present invention. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, the poly-A tail contained in an RNA molecule described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly-A tail at its 3′-end, i.e., the poly-A tail is not masked or followed at its 3′-end by a nucleotide other than A.

In some embodiments, the poly-A tail may comprise at least 20, at least 30, at least 40, at least 80, or at least 100 and up to 500, up to 400, up to 300, up to 200, or up to 150 nucleotides. In some embodiments, the poly-A tail may essentially consist of at least 20, at least 30, at least 40, at least 80, or at least 100 and up to 500, up to 400, up to 300, up to 200, or up to 150 nucleotides. In some embodiments, the poly-A tail may consist of at least 20, at least 30, at least 40, at least 80, or at least 100 and up to 500, up to 400, up to 300, up to 200, or up to 150 nucleotides. In some embodiments, the poly-A tail comprises at least 100 nucleotides. In some embodiments, the poly-A tail comprises about 150 nucleotides. In some embodiments, the poly-A tail comprises about 120 nucleotides.

According to the disclosure, vaccine antigen is preferably administered as single-stranded, 5′-capped mRNA that is translated into the respective protein upon entering antigen-presenting cells (APCs). Preferably, the RNA contains structural elements optimized for maximal efficacy of the RNA with respect to stability and translational efficiency (5′-cap, 5′-UTR, 3′-UTR, poly(A)-tail).

In one embodiment, beta-S-ARCA(D1) is utilized as specific capping structure at the 5′-end of the RNA. In one embodiment, the 5′-UTR sequence is derived from the human alpha-globin mRNA. In one embodiment, two re-iterated 3′-UTRs derived from the human beta-globin mRNA are placed between the coding sequence and the poly(A)-tail to assure higher maximum protein levels and prolonged persistence of the mRNA. In one embodiment, a poly(A)-tail measuring 110 nucleotides in length, consisting of a stretch of 30 adenosine residues, followed by a 10 nucleotide linker sequence and another 70 adenosine residues is used. This poly(A)-tail sequence was designed to enhance RNA stability and translational efficiency in dendritic cells.

The RNA is preferably administered as lipoplex particles, preferably comprising DOTMA and DOPE, as further described below. Such particles are preferably administered by systemic administration, in particular by intravenous administration.

In the context of the present disclosure, the term “transcription” relates to a process, wherein the genetic code in a DNA sequence is transcribed into RNA. Subsequently, the RNA may be translated into peptide or protein.

With respect to RNA, the term “expression” or “translation” relates to the process in the ribosomes of a cell by which a strand of mRNA directs the assembly of a sequence of amino acids to make a peptide or protein.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

According to the disclosure, the term “RNA encodes” means that the RNA, if present in the appropriate environment, such as within cells of a target tissue, can direct the assembly of amino acids to produce the peptide or protein it encodes during the process of translation. In one embodiment, RNA is able to interact with the cellular translation machinery allowing translation of the peptide or protein. A cell may produce the encoded peptide or protein intracellularly (e.g. in the cytoplasm and/or in the nucleus), may secrete the encoded peptide or protein, or may produce it on the surface.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence. Expression can be transient or stable. According to the invention, the term expression also includes an “aberrant expression” or “abnormal expression”.

As used herein, the terms “linked,” “fused”, or “fusion” are used interchangeably. These terms refer to the joining together of two or more elements or components or domains.

Cytokines

The methods described herein may comprise providing to a subject one or more cytokines, e.g., by administering to the subject the one or more cytokines, a polynucleotide encoding the one or more cytokines or a host cell expressing the one or more cytokines.

The term “cytokine” as used herein includes naturally occurring cytokines and functional variants thereof (including fragments of the naturally occurring cytokines and variants thereof). One particularly preferred cytokine is IL2.

Cytokines are a category of small proteins (˜5-20 kDa) that are important in cell signaling. Their release has an effect on the behavior of cells around them. Cytokines are involved in autocrine signaling, paracrine signaling and endocrine signaling as immunomodulating agents. Cytokines include chemokines, interferons, interleukins, lymphokines, and tumour necrosis factors but generally not hormones or growth factors (despite some overlap in the terminology). Cytokines are produced by a broad range of cells, including immune cells like macrophages, B lymphocytes, T lymphocytes and mast cells, as well as endothelial cells, fibroblasts, and various stromal cells. A given cytokine may be produced by more than one type of cell. Cytokines act through receptors, and are especially important in the immune system; cytokines modulate the balance between humoral and cell-based immune responses, and they regulate the maturation, growth, and responsiveness of particular cell populations. Some cytokines enhance or inhibit the action of other cytokines in complex ways.

IL2

Interleukin-2 (IL2) is a cytokine that induces proliferation of antigen-activated T cells and stimulates natural killer (NK) cells. The biological activity of IL2 is mediated through a multi-subunit IL2 receptor complex (IL2R) of three polypeptide subunits that span the cell membrane: p55 (IL2Rα, the alpha subunit, also known as CD25 in humans), p75 (IL2Rβ, the beta subunit, also known as CD122 in humans) and p64 (IL2Rγ, the gamma subunit, also known as CD132 in humans). T cell response to IL2 depends on a variety of factors, including: (1) the concentration of IL2; (2) the number of IL2R molecules on the cell surface; and (3) the number of IL2R occupied by IL2 (i.e., the affinity of the binding interaction between IL2 and IL2R (Smith, “Cell Growth Signal Transduction is Quantal” In Receptor Activation by Antigens, Cytokines, Hormones, and Growth Factors 766:263-271, 1995)). The IL2:IL2R complex is internalized upon ligand binding and the different components undergo differential sorting. When administered as an intravenous (i.v.) bolus, IL2 has a rapid systemic clearance (an initial clearance phase with a half-life of 12.9 minutes followed by a slower clearance phase with a half-life of 85 minutes) (Konrad et al., Cancer Res. 50:2009-2017, 1990).

In eukaryotic cells human IL2 is synthesized as a precursor polypeptide of 153 amino acids, from which 20 amino acids are removed to generate mature secreted IL2. Recombinant human IL2 has been produced in E. coli, in insect cells and in mammalian COS cells.

According to the disclosure, IL2 (optionally as a portion of extended-PK IL2) may be naturally occurring IL2 or a fragment or variant thereof. IL2 may be human IL2 and may be derived from any vertebrate, especially any mammal.

Extended-PK Group

Cytokine polypeptides described herein can be prepared as fusion or chimeric polypeptides that include a cytokine portion and a heterologous polypeptide (i.e., a polypeptide that is not a cytokine or a variant thereof). The resulting molecule, hereafter referred to as “extended-pharmacokinetic (PK) cytokine,” has a prolonged circulation half-life relative to free cytokine. The prolonged circulation half-life of extended-PK cytokine permits in vivo serum cytokine concentrations to be maintained within a therapeutic range, potentially leading to the enhanced activation of many types of immune cells, including T cells. Because of its favorable pharmacokinetic profile, extended-PK cytokine can be dosed less frequently and for longer periods of time when compared with unmodified cytokine.

As used herein, “half-life” refers to the time taken for the serum or plasma concentration of a compound such as a peptide or protein to reduce by 50%, in vivo, for example due to degradation and/or clearance or sequestration by natural mechanisms. An extended-PK cytokine such as an extended-PK interleukin (IL) suitable for use herein is stabilized in vivo and its half-life increased by, e.g., fusion to serum albumin (e.g., HSA or MSA), which resist degradation and/or clearance or sequestration. The half-life can be determined in any manner known per se, such as by pharmacokinetic analysis. Suitable techniques will be clear to the person skilled in the art, and may for example generally involve the steps of suitably administering a suitable dose of the amino acid sequence or compound to a subject; collecting blood samples or other samples from said subject at regular intervals; determining the level or concentration of the amino acid sequence or compound in said blood sample; and calculating, from (a plot of) the data thus obtained, the time until the level or concentration of the amino acid sequence or compound has been reduced by 50% compared to the initial level upon dosing. Further details are provided in, e.g., standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2nd Rev. Edition, Marcel Dekker (1982).

The cytokine may be fused to an extended-PK group, which increases circulation half-life. Non-limiting examples of extended-PK groups are described infra. It should be understood that other PK groups that increase the circulation half-life of cytokines, or variants thereof, are also applicable to the present disclosure. In certain embodiments, the extended-PK group is a serum albumin domain (e.g., mouse serum albumin, human serum albumin).

As used herein, the term “PK” is an acronym for “pharmacokinetic” and encompasses properties of a compound including, by way of example, absorption, distribution, metabolism, and elimination by a subject. As used herein, an “extended-PK group” refers to a protein, peptide, or moiety that increases the circulation half-life of a biologically active molecule when fused to or administered together with the biologically active molecule. Examples of an extended-PK group include serum albumin (e.g., HSA), Immunoglobulin Fc or Fc fragments and variants thereof, transferrin and variants thereof, and human serum albumin (HSA) binders (as disclosed in U.S. Publication Nos. 2005/0287153 and 2007/0003549). Other exemplary extended-PK groups are disclosed in Kontermann, Expert Opin Biol Ther, 2016 July; 16(7):903-15 which is herein incorporated by reference in its entirety. As used herein, an “extended-PK cytokine” refers to a cytokine moiety in combination with an extended-PK group. In one embodiment, the extended-PK cytokine is a fusion protein in which a cytokine moiety is linked or fused to an extended-PK group. As used herein, an “extended-PK IL” refers to an interleukin (IL) moiety (including an IL variant moiety) in combination with an extended-PK group. In one embodiment, the extended-PK IL is a fusion protein in which an IL moiety is linked or fused to an extended-PK group. An exemplary fusion protein is an HSA/IL2 fusion in which an IL2 moiety is fused with HSA.

In certain embodiments, the serum half-life of an extended-PK cytokine is increased relative to the cytokine alone (i.e., the cytokine not fused to an extended-PK group). In certain embodiments, the serum half-life of the extended-PK cytokine is at least 20, 40, 60, 80, 100, 120, 150, 180, 200, 400, 600, 800, or 1000% longer relative to the serum half-life of the cytokine alone. In certain embodiments, the serum half-life of the extended-PK cytokine is at least 1.5-fold, 2-fold, 2.5-fold, 3-fold, 3.5 fold, 4-fold, 4.5-fold, 5-fold, 6-fold, 7-fold, 8-fold, 10- fold, 12-fold, 13-fold, 15-fold, 17-fold, 20-fold, 22- fold, 25-fold, 27-fold, 30-fold, 35-fold, 40-fold, or 50-fold greater than the serum half-life of the cytokine alone. In certain embodiments, the serum half-life of the extended-PK cytokine is at least 10 hours, 15 hours, 20 hours, 25 hours, 30 hours, 35 hours, 40 hours, 50 hours, 60 hours, 70 hours, 80 hours, 90 hours, 100 hours, 110 hours, 120 hours, 130 hours, 135 hours, 140 hours, 150 hours, 160 hours, or 200 hours.

In certain embodiments, the extended-PK group includes serum albumin, or fragments thereof or variants of the serum albumin or fragments thereof (all of which for the purpose of the present disclosure are comprised by the term “albumin”). Polypeptides described herein may be fused to albumin (or a fragment or variant thereof) to form albumin fusion proteins. Such albumin fusion proteins are described in U.S. Publication No. 20070048282.

As used herein, “albumin fusion protein” refers to a protein formed by the fusion of at least one molecule of albumin (or a fragment or variant thereof) to at least one molecule of a protein such as a therapeutic protein, in particular IL2 (or variant thereof). The albumin fusion protein may be generated by translation of a nucleic acid in which a polynucleotide encoding a therapeutic protein is joined in-frame with a polynucleotide encoding an albumin. The therapeutic protein and albumin, once part of the albumin fusion protein, may each be referred to as a “portion”, “region” or “moiety” of the albumin fusion protein (e.g., a “therapeutic protein portion” or an “albumin protein portion”). In a highly preferred embodiment, an albumin fusion protein comprises at least one molecule of a therapeutic protein (including, but not limited to a mature form of the therapeutic protein) and at least one molecule of albumin (including but not limited to a mature form of albumin). In one embodiment, an albumin fusion protein is processed by a host cell such as a cell of the target organ for administered RNA, e.g. a liver cell, and secreted into the circulation. Processing of the nascent albumin fusion protein that occurs in the secretory pathways of the host cell used for expression of the RNA may include, but is not limited to signal peptide cleavage; formation of disulfide bonds; proper folding; addition and processing of carbohydrates (such as for example, N- and O-linked glycosylation); specific proteolytic cleavages; and/or assembly into multimeric proteins. An albumin fusion protein is preferably encoded by RNA in a non-processed form which in particular has a signal peptide at its N-terminus and following secretion by a cell is preferably present in the processed form wherein in particular the signal peptide has been cleaved off. In a most preferred embodiment, the “processed form of an albumin fusion protein” refers to an albumin fusion protein product which has undergone N-terminal signal peptide cleavage, herein also referred to as a “mature albumin fusion protein”.

In preferred embodiments, albumin fusion proteins comprising a therapeutic protein have a higher plasma stability compared to the plasma stability of the same therapeutic protein when not fused to albumin. Plasma stability typically refers to the time period between when the therapeutic protein is administered in vivo and carried into the bloodstream and when the therapeutic protein is degraded and cleared from the bloodstream, into an organ, such as the kidney or liver that ultimately clears the therapeutic protein from the body. Plasma stability is calculated in terms of the half-life of the therapeutic protein in the bloodstream. The half-life of the therapeutic protein in the bloodstream can be readily determined by common assays known in the art.

As used herein, “albumin” refers collectively to albumin protein or amino acid sequence, or an albumin fragment or variant, having one or more functional activities (e.g., biological activities) of albumin. In particular, “albumin” refers to human albumin or fragments or variants thereof especially the mature form of human albumin, or albumin from other vertebrates or fragments thereof, or variants of these molecules. The albumin may be derived from any vertebrate, especially any mammal, for example human, mouse, cow, sheep, or pig. Non-mammalian albumins include, but are not limited to, hen and salmon. The albumin portion of the albumin fusion protein may be from a different animal than the therapeutic protein portion.

In certain embodiments, the albumin is human serum albumin (HSA), or fragments or variants thereof, such as those disclosed in U.S. Pat. No. 5,876,969, WO 2011/124718, WO 2013/075066, and WO 2011/0514789.

The terms, human serum albumin (HSA) and human albumin (HA) are used interchangeably herein. The terms, “albumin and “serum albumin” are broader, and encompass human serum albumin (and fragments and variants thereof) as well as albumin from other species (and fragments and variants thereof).

As used herein, a fragment of albumin sufficient to prolong the therapeutic activity or plasma stability of the therapeutic protein refers to a fragment of albumin sufficient in length or structure to stabilize or prolong the therapeutic activity or plasma stability of the protein so that the plasma stability of the therapeutic protein portion of the albumin fusion protein is prolonged or extended compared to the plasma stability in the non-fusion state.

The albumin portion of the albumin fusion proteins may comprise the full length of the albumin sequence, or may include one or more fragments thereof that are capable of stabilizing or prolonging the therapeutic activity or plasma stability. Such fragments may be of 10 or more amino acids in length or may include about 15, 20, 25, 30, 50, or more contiguous amino acids from the albumin sequence or may include part or all of specific domains of albumin. For instance, one or more fragments of HSA spanning the first two immunoglobulin-like domains may be used. In a preferred embodiment, the HSA fragment is the mature form of HSA.

Generally speaking, an albumin fragment or variant will be at least 100 amino acids long, preferably at least 150 amino acids long.

According to the disclosure, albumin may be naturally occurring albumin or a fragment or variant thereof. Albumin may be human albumin and may be derived from any vertebrate, especially any mammal.

Preferably, the albumin fusion protein comprises albumin as the N-terminal portion, and a therapeutic protein as the C-terminal portion. Alternatively, an albumin fusion protein comprising albumin as the C-terminal portion, and a therapeutic protein as the N-terminal portion may also be used.

In one embodiment, the therapeutic protein(s) is (are) joined to the albumin through (a) peptide linker(s). A linker peptide between the fused portions may provide greater physical separation between the moieties and thus maximize the accessibility of the therapeutic protein portion, for instance, for binding to its cognate receptor. The linker peptide may consist of amino acids such that it is flexible or more rigid. The linker sequence may be cleavable by a protease or chemically.

As used herein, the term “Fc region” refers to the portion of a native immunoglobulin formed by the respective Fc domains (or Fc moieties) of its two heavy chains. As used herein, the term “Fc domain” refers to a portion or fragment of a single immunoglobulin (Ig) heavy chain wherein the Fc domain does not comprise an Fv domain. In certain embodiments, an Fc domain begins in the hinge region just upstream of the papain cleavage site and ends at the C-terminus of the antibody. Accordingly, a complete Fc domain comprises at least a hinge domain, a CH2 domain, and a CH3 domain. In certain embodiments, an Fc domain comprises at least one of: a hinge (e.g., upper, middle, and/or lower hinge region) domain, a CH2 domain, a CH3 domain, a CH4 domain, or a variant, portion, or fragment thereof. In certain embodiments, an Fc domain comprises a complete Fc domain (i.e., a hinge domain, a CH2 domain, and a CH3 domain). In certain embodiments, an Fc domain comprises a hinge domain (or portion thereof) fused to a CH3 domain (or portion thereof). In certain embodiments, an Fc domain comprises a CH2 domain (or portion thereof) fused to a CH3 domain (or portion thereof). In certain embodiments, an Fc domain consists of a CH3 domain or portion thereof. In certain embodiments, an Fc domain consists of a hinge domain (or portion thereof) and a CH3 domain (or portion thereof). In certain embodiments, an Fc domain consists of a CH2 domain (or portion thereof) and a CH3 domain. In certain embodiments, an Fc domain consists of a hinge domain (or portion thereof) and a CH2 domain (or portion thereof). In certain embodiments, an Fc domain lacks at least a portion of a CH2 domain (e.g., all or part of a CH2 domain). An Fc domain herein generally refers to a polypeptide comprising all or part of the Fc domain of an immunoglobulin heavy-chain. This includes, but is not limited to, polypeptides comprising the entire CH1, hinge, CH2, and/or CH3 domains as well as fragments of such peptides comprising only, e.g., the hinge, CH2, and CH3 domain. The Fc domain may be derived from an immunoglobulin of any species and/or any subtype, including, but not limited to, a human IgG1, IgG2, IgG3, IgG4, IgD, IgA, IgE, or IgM antibody. The Fc domain encompasses native Fc and Fc variant molecules. As set forth herein, it will be understood by one of ordinary skill in the art that any Fc domain may be modified such that it varies in amino acid sequence from the native Fc domain of a naturally occurring immunoglobulin molecule. In certain embodiments, the Fc domain has reduced effector function (e.g., FcγR binding).

The Fc domains of a polypeptide described herein may be derived from different immunoglobulin molecules. For example, an Fc domain of a polypeptide may comprise a CH2 and/or CH3 domain derived from an IgG1 molecule and a hinge region derived from an IgG3 molecule. In another example, an Fc domain can comprise a chimeric hinge region derived, in part, from an IgG1 molecule and, in part, from an IgG3 molecule. In another example, an Fc domain can comprise a chimeric hinge derived, in part, from an IgG1 molecule and, in part, from an IgG4 molecule.

In certain embodiments, an extended-PK group includes an Fc domain or fragments thereof or variants of the Fc domain or fragments thereof (all of which for the purpose of the present disclosure are comprised by the term “Fc domain”). The Fc domain does not contain a variable region that binds to antigen. Fc domains suitable for use in the present disclosure may be obtained from a number of different sources. In certain embodiments, an Fc domain is derived from a human immunoglobulin. In certain embodiments, the Fc domain is from a human IgG1 constant region. It is understood, however, that the Fc domain may be derived from an immunoglobulin of another mammalian species, including for example, a rodent (e.g. a mouse, rat, rabbit, guinea pig) or non-human primate (e.g. chimpanzee, macaque) species.

Moreover, the Fc domain (or a fragment or variant thereof) may be derived from any immunoglobulin class, including IgM, IgG, IgD, IgA, and IgE, and any immunoglobulin isotype, including IgG1, IgG2, IgG3, and IgG4.

A variety of Fc domain gene sequences (e.g., mouse and human constant region gene sequences) are available in the form of publicly accessible deposits. Constant region domains comprising an Fc domain sequence can be selected lacking a particular effector function and/or with a particular modification to reduce immunogenicity. Many sequences of antibodies and antibody-encoding genes have been published and suitable Fc domain sequences (e.g. hinge, CH2, and/or CH3 sequences, or fragments or variants thereof) can be derived from these sequences using art recognized techniques.

In certain embodiments, the extended-PK group is a serum albumin binding protein such as those described in US2005/0287153, US2007/0003549, US2007/0178082, US2007/0269422, US2010/0113339, WO2009/083804, and WO2009/133208, which are herein incorporated by reference in their entirety. In certain embodiments, the extended-PK group is transferrin, as disclosed in U.S. Pat. Nos. 7,176,278 and 8,158,579, which are herein incorporated by reference in their entirety. In certain embodiments, the extended-PK group is a serum immunoglobulin binding protein such as those disclosed in US2007/0178082, US2014/0220017, and US2017/0145062, which are herein incorporated by reference in their entirety. In certain embodiments, the extended-PK group is a fibronectin (Fn)-based scaffold domain protein that binds to serum albumin, such as those disclosed in US2012/0094909, which is herein incorporated by reference in its entirety. Methods of making fibronectin-based scaffold domain proteins are also disclosed in US2012/0094909. A non-limiting example of a Fn3-based extended-PK group is Fn3(HSA), i.e., a Fn3 protein that binds to human serum albumin.

In certain aspects, the extended-PK cytokine, suitable for use according to the disclosure, can employ one or more peptide linkers. As used herein, the term “peptide linker” refers to a peptide or polypeptide sequence which connects two or more domains (e.g., the extended-PK moiety and an IL moiety such as IL2) in a linear amino acid sequence of a polypeptide chain. For example, peptide linkers may be used to connect a cytokine moiety to a HSA domain.

Linkers suitable for fusing the extended-PK group to e.g. IL2 are well known in the art. Exemplary linkers include glycine-serine-polypeptide linkers, glycine-proline-polypeptide linkers, and proline-alanine polypeptide linkers. In certain embodiments, the linker is a glycine-serine-polypeptide linker, i.e., a peptide that consists of glycine and serine residues.

In addition to, or in place of, the heterologous polypeptides described above, a cytokine variant polypeptide described herein can contain sequences encoding a “marker” or “reporter”. Examples of marker or reporter genes include β-lactamase, chloramphenicol acetyltransferase (CAT), adenosine deaminase (ADA), aminoglycoside phosphotransferase, dihydrofolate reductase (DHFR), hygromycin-B-hosphotransferase (HPH), thymidine kinase (TK), β-galactosidase, and xanthine guanine phosphoribosyltransferase (XGPRT).

Antigen Receptors

Cells described herein such as immune effector cells may express an antigen receptor such as a chimeric antigen receptor (CAR) or a T cell receptor (TCR) binding antigen or a procession product thereof, in particular when present on or presented by a target cell. Cells may naturally express an antigen receptor or be modified (e.g., ex vivo/in vitro or in vivo in a subject to be treated) to express an antigen receptor. In one embodiment, modification to express an antigen receptor takes place ex vivo/in vitro. Subsequently, modified cells may be administered to a patient. In one embodiment, modification to express an antigen receptor takes place in vivo. The cells may be endogenous cells of the patient or may have been administered to a patient.

Chimeric Antigen Receptors

Adoptive cell transfer therapy with CAR-engineered T cells expressing chimeric antigen receptors is a promising anti-cancer therapeutic as CAR-modified T cells can be engineered to target virtually any tumor antigen. For example, patient's T cells may be genetically engineered (genetically modified) to express CARs specifically directed towards antigens on the patient's tumor cells, then infused back into the patient.

According to the invention, the term “CAR” (or “chimeric antigen receptor”) is synonymous with the terms “chimeric T cell receptor” and “artificial T cell receptor” and relates to an artificial receptor comprising a single molecule or a complex of molecules which recognizes, i.e. binds to, a target structure (e.g. an antigen) on a target cell such as a cancer cell (e.g. by binding of an antigen binding domain to an antigen expressed on the surface of the target cell) and may confer specificity onto an immune effector cell such as a T cell expressing said CAR on the cell surface. Such cells do not necessarily require processing and presentation of an antigen for recognition of the target cell but rather may recognize preferably with specificity any antigen present on a target cell. Preferably, recognition of the target structure by a CAR results in activation of an immune effector cell expressing said CAR. A CAR may comprise one or more protein units said protein units comprising one or more domains as described herein. The term “CAR” does not include T cell receptors.

A CAR comprises a target-specific binding element otherwise referred to as an antigen binding moiety or antigen binding domain that is generally part of the extracellular domain of the CAR. The antigen binding domain recognizes a ligand that acts as a cell surface marker on target cells associated with a particular disease state. Specifically, the CAR of the invention targets the antigen such as tumor antigen on a diseased cell such as tumor cell.

In one embodiment, the binding domain in the CAR binds specifically to the antigen. In one embodiment, the antigen to which the binding domain in the CAR binds is expressed in a cancer cell (tumor antigen). In one embodiment, the antigen is expressed on the surface of a cancer cell. In one embodiment, the binding domain binds to an extracellular domain or to an epitope in an extracellular domain of the antigen. In one embodiment, the binding domain binds to native epitopes of the antigen present on the surface of living cells.

In one embodiment of the invention, an antigen binding domain comprises a variable region of a heavy chain of an immunoglobulin (VH) with a specificity for the antigen and a variable region of a light chain of an immunoglobulin (VL) with a specificity for the antigen. In one embodiment, an immunoglobulin is an antibody. In one embodiment, said heavy chain variable region (VH) and the corresponding light chain variable region (VL) are connected via a peptide linker. Preferably, the antigen binding moiety portion in the CAR is a scFv.

The CAR is designed to comprise a transmembrane domain that is fused to the extracellular domain of the CAR. In one embodiment, the transmembrane domain is not naturally associated with one of the domains in the CAR. In one embodiment, the transmembrane domain is naturally associated with one of the domains in the CAR. In one embodiment, the transmembrane domain is modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain.

In some instances, the CAR of the invention comprises a hinge domain which forms the linkage between the transmembrane domain and the extracellular domain.

The cytoplasmic domain or otherwise the intracellular signaling domain of the CAR is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

In one embodiment, the CAR comprises a primary cytoplasmic signaling sequence derived from CD3-zeta. Further, the cytoplasmic domain of the CAR may comprise the CD3-zeta signaling domain combined with a costimulatory signaling region.

The identity of the co-stimulation domain is limited only in that it has the ability to enhance cellular proliferation and survival upon binding of the targeted moiety by the CAR. Suitable co-stimulation domains include CD28, CD137 (4-1BB), a member of the tumor necrosis factor receptor (TNFR) superfamily, CD134 (OX40), a member of the TNFR-superfamily of receptors, and CD278 (ICOS), a CD28-superfamily co-stimulatory molecule expressed on activated T cells. The skilled person will understand that sequence variants of these noted co-stimulation domains can be used without adversely impacting the invention, where the variants have the same or similar activity as the domain on which they are modeled. Such variants will have at least about 80% sequence identity to the amino acid sequence of the domain from which they are derived. In some embodiments of the invention, the CAR constructs comprise two co-stimulation domains. While the particular combinations include all possible variations of the four noted domains, specific examples include CD28+CD137 (4-1BB) and CD28+CD134 (OX40).

The cytoplasmic signaling sequences within the cytoplasmic signaling portion of the CAR may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the CAR comprises a signal peptide which directs the nascent protein into the endoplasmic reticulum. In one embodiment, the signal peptide precedes the antigen binding domain. In one embodiment, the signal peptide is derived from an immunoglobulin such as IgG.

A CAR may comprise the above domains, together in the form of a fusion protein. Such fusion proteins will generally comprise an antigen binding domain, one or more co-stimulation domains, and a signaling sequence, linked in a N-terminal to C-terminal direction. However, the CARs of the present invention are not limited to this arrangement and other arrangements are acceptable and include a binding domain, a signaling domain, and one or more co-stimulation domains. It will be understood that because the binding domain must be free to bind antigen, the placement of the binding domain in the fusion protein will generally be such that display of the region on the exterior of the cell is achieved. In the same manner, because the co-stimulation and signaling domains serve to induce activity and proliferation of the cytotoxic lymphocytes, the fusion protein will generally display these two domains in the interior of the cell.

In one embodiment, a CAR molecule comprises:

i) a target antigen (e.g., CLDN6 or CLDN18.2) binding domain;

ii) a transmembrane domain; and

iii) an intracellular domain that comprises a 4-1BB costimulatory domain, and a CD3-zeta signaling domain.

In one embodiment, the antigen binding domain comprises an scFv. In one embodiment, the transmembrane domain comprises a transmembrane domain of a protein selected from the group consisting of the alpha, beta or zeta chain of the T cell receptor, CD28, CD3 epsilon, CD45, CD4, CD5, CD8, CD9, CD16, CD22, CD33, CD37, CD64, CD80, CD86, CD134, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Ra, ITGA1, VLA1, CD49a, ITGA4, 1A4, CD49D, ITGA6, VLA-6, CD49f, ITGAD, CD11d, ITGAE, CD103, ITGAL, CD11a, LFA-1, ITGAM, CD11b, ITGAX, CD11c, ITGBI, CD29, ITGB2, CD18, LFA-1, ITGB7, TNFR2, DNAMI (CD226), SLAMF4 (CD244, 2B4), CD84, CD96 (Tactile), CEACAM1, CRT AM, Ly9 (CD229), CD160 (BY55), PSGLI, CDIOO (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof. In one embodiment, the transmembrane domain comprises a CD8a transmembrane domain. In one embodiment, the antigen binding domain is connected to the transmembrane domain by a hinge domain. In one embodiment, the hinge domain is a CD8a hinge domain.

In one embodiment, the CAR molecule of the invention comprises:

i) a target antigen binding domain;

ii) a CD8a hinge domain;

iii) a CD8a transmembrane domain; and

iv) an intracellular domain that comprises a 4-1BB costimulatory domain, and a CD3-zeta signaling domain.

The term “antibody” includes an immunoglobulin comprising at least two heavy (H) chains and two light (L) chains inter-connected by disulfide bonds. Each heavy chain is comprised of a heavy chain variable region (abbreviated herein as VH) and a heavy chain constant region. Each light chain is comprised of a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. An antibody binds, preferably specifically binds with an antigen. Antibodies can be intact immunoglobulins derived from natural sources or from recombinant sources and can be immunoreactive portions or fragments of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab′)2, as well as single chain antibodies and humanized antibodies (Harlow et al., 1999, In: Using Antibodies: A Laboratory Manual, Cold Spring Harbor Laboratory Press, NY; Harlow et al., 1989, in: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “antibody fragment” refers to a portion of an intact antibody and typically comprises the antigenic determining variable regions of an intact antibody.

Examples of antibody fragments include, but are not limited to, Fab, Fab′, F(ab′)₂, and Fv fragments, linear antibodies, scFv antibodies, and multispecific antibodies formed from antibody fragments.

An “antibody heavy chain”, as used herein, refers to the larger of the two types of polypeptide chains present in antibody molecules in their naturally occurring conformations.

An “antibody light chain”, as used herein, refers to the smaller of the two types of polypeptide chains present in antibody molecules in their naturally occurring conformations, K and A light chains refer to the two major antibody light chain isotypes.

According to the disclosure, a CAR which when present on a T cell recognizes an antigen such as on the surface of antigen presenting cells or diseased cells such as cancer cells, such that the T cell is stimulated, and/or expanded or exerts effector functions as described above.

Genetic Modification of Immune Effector Cells

A variety of methods may be used to introduce antigen receptors such as CAR constructs into cells such as T cells to produce cells genetically modified to express the antigen receptors. Such methods including non-viral-based DNA transfection, non-viral-based RNA transfection, e.g., mRNA transfection, transposon-based systems, and viral-based systems. Non-viral-based DNA transfection has low risk of insertional mutagenesis. Transposon-based systems can integrate transgenes more efficiently than plasmids that do not contain an integrating element. Viral-based systems include the use of y-retroviruses and lentiviral vectors. y-Retroviruses are relatively easy to produce, efficiently and permanently transduce T cells, and have preliminarily proven safe from an integration standpoint in primary human T cells. Lentiviral vectors also efficiently and permanently transduce T cells but are more expensive to manufacture. They are also potentially safer than retrovirus based systems.

In one embodiment of all aspects of the invention, T cells or T cell progenitors are transfected either ex vivo or in vivo with nucleic acid encoding the antigen receptor. In one embodiment, a combination of ex vivo and in vivo transfection may be used. In one embodiment of all aspects of the invention, the T cells or T cell progenitors are from the subject to be treated. In one embodiment of all aspects of the invention, the T cells or T cell progenitors are from a subject which is different to the subject to be treated.

CAR T cells may be produced in vivo, and therefore nearly instantaneously, using nanoparticles targeted to T cells. For example, poly(β-amino ester)-based nanoparticles may be coupled to anti-CD3e F(ab) fragments for binding to CD3 on T cells. Upon binding to T cells, these nanoparticles are endocytosed. Their contents, for example plasmid DNA encoding an anti-tumor antigen CAR, may be directed to the T cell nucleus due to the inclusion of peptides containing microtubule-associated sequences (MTAS) and nuclear localization signals (NLSs). The inclusion of transposons flanking the CAR gene expression cassette and a separate plasmid encoding a hyperactive transposase, may allow for the efficient integration of the CAR vector into chromosomes. Such system that allows for the in vivo production of CART cells following nanoparticle infusion is described in Smith et al. (2017) Nat. Nanotechnol. 12:813-820.

Furthermore, CD19-CAR T cells can be generated directly in vivo using the lentiviral vector CD8-LV specifically targeting human CD8⁺ cells (Pfeiffer A. et al., EMBO Mol. Med. November; 10(11), 2018, 9158).

Another possibility is to use the CRISPR/Cas9 method to deliberately place a CAR coding sequence at a specific locus. For example, existing T cell receptors (TCR) may be knocked out, while knocking in the CAR and placing it under the dynamic regulatory control of the endogenous promoter that would otherwise moderate TCR expression; c.f., e.g., Eyquem et al. (2017) Nature 543:113-117.

In one embodiment of all aspects of the invention, the cells genetically modified to express an antigen receptor are stably or transiently transfected with nucleic acid encoding the antigen receptor. Thus, the nucleic acid encoding the antigen receptor is integrated or not integrated into the genome of the cells.

In one embodiment of all aspects of the invention, the cells genetically modified to express an antigen receptor are inactivated for expression of an endogenous T cell receptor and/or endogenous HLA.

In one embodiment of all aspects of the invention, the cells described herein may be autologous, allogeneic or syngeneic to the subject to be treated. In one embodiment, the present disclosure envisions the removal of cells from a patient and the subsequent re-delivery of the cells to the patient. In one embodiment, the present disclosure does not envision the removal of cells from a patient. In the latter case all steps of genetic modification of cells are performed in vivo.

The term “autologous” is used to describe anything that is derived from the same subject. For example, “autologous transplant” refers to a transplant of tissue or organs derived from the same subject. Such procedures are advantageous because they overcome the immunological barrier which otherwise results in rejection.

The term “allogeneic” is used to describe anything that is derived from different individuals of the same species. Two or more individuals are said to be allogeneic to one another when the genes at one or more loci are not identical.

The term “syngeneic” is used to describe anything that is derived from individuals or tissues having identical genotypes, i.e., identical twins or animals of the same inbred strain, or their tissues.

The term “heterologous” is used to describe something consisting of multiple different elements. As an example, the transfer of one individual's bone marrow into a different individual constitutes a heterologous transplant. A heterologous gene is a gene derived from a source other than the subject.

Antigen

The methods described herein further comprise the step of contacting the immune effector cells, in particular immune effector cells expressing an antigen receptor, e.g., immune effector cells which are genetically manipulated to express an antigen receptor, in the subject being treated, with a cognate antigen molecule (also referred herein to as “antigen targeted by the antigen receptor”, “vaccine antigen” or simply “antigen”), wherein the antigen molecule or a procession product thereof, e.g., a fragment thereof, binds to the antigen receptor such as TCR or CAR carried by the immune effector cells. In one embodiment, the cognate antigen molecule comprises the antigen expressed by a target cell to which the immune effector cells are targeted or a fragment thereof, or a variant of the antigen or the fragment.

Accordingly, the methods described herein comprise the step of administering the cognate antigen molecule, a nucleic acid coding therefor or cells expressing the cognate antigen molecule to the subject. In one embodiment, the nucleic acid encoding the cognate antigen molecule is expressed in cells of the subject to provide the cognate antigen molecule. In one embodiment, expression of the cognate antigen molecule is at the cell surface. In one embodiment, the nucleic acid encoding the cognate antigen molecule is transiently expressed in cells of the subject. In one embodiment, the nucleic encoding the cognate antigen molecule is RNA. In one embodiment, the cognate antigen molecule or the nucleic acid coding therefor is administered systemically. In one embodiment, after systemic administration of the nucleic acid encoding the cognate antigen molecule, expression of the nucleic acid encoding the cognate antigen molecule in spleen occurs. In one embodiment, after systemic administration of the nucleic acid encoding the cognate antigen molecule, expression of the nucleic acid encoding the cognate antigen molecule in antigen presenting cells, preferably professional antigen presenting cells occurs. In one embodiment, the antigen presenting cells are selected from the group consisting of dendritic cells, macrophages and B cells. In one embodiment, after systemic administration of the nucleic acid encoding the cognate antigen molecule, no or essentially no expression of the nucleic acid encoding the cognate antigen molecule in lung and/or liver occurs. In one embodiment, after systemic administration of the nucleic acid encoding the cognate antigen molecule, expression of the nucleic acid encoding the cognate antigen molecule in spleen is at least 5-fold the amount of expression in lung.

A peptide and protein antigen which is provided to a subject according to the invention (either by administering the peptide and protein antigen, a nucleic acid, in particular RNA, encoding the peptide and protein antigen or cells expressing the peptide and protein antigen), i.e., a vaccine antigen, preferably results in stimulation, priming and/or expansion of immune effector cells in the subject being administered the peptide or protein antigen, nucleic acid or cells. Said stimulated, primed and/or expanded immune effector cells are preferably directed against a target antigen, in particular a target antigen expressed by diseased cells, tissues and/or organs, i.e., a disease-associated antigen. Thus, a vaccine antigen may comprise the disease-associated antigen, or a fragment or variant thereof. In one embodiment, such fragment or variant is immunologically equivalent to the disease-associated antigen. In the context of the present disclosure, the term “fragment of an antigen” or “variant of an antigen” means an agent which results in stimulation, priming and/or expansion of immune effector cells which stimulated, primed and/or expanded immune effector cells target the antigen, i.e. a disease-associated antigen, in particular when presented by diseased cells, tissues and/or organs. Thus, the vaccine antigen may correspond to or may comprise the disease-associated antigen, may correspond to or may comprise a fragment of the disease-associated antigen or may correspond to or may comprise an antigen which is homologous to the disease-associated antigen or a fragment thereof. If the vaccine antigen comprises a fragment of the disease-associated antigen or an amino acid sequence which is homologous to a fragment of the disease-associated antigen said fragment or amino acid sequence may comprise an epitope of the disease-associated antigen to which the antigen receptor of the immune effector cells is targeted or a sequence which is homologous to an epitope of the disease-associated antigen. Thus, according to the disclosure, a vaccine antigen may comprise an immunogenic fragment of a disease-associated antigen or an amino acid sequence being homologous to an immunogenic fragment of a disease-associated antigen. An “immunogenic fragment of an antigen” according to the disclosure preferably relates to a fragment of an antigen which is capable of stimulating, priming and/or expanding immune effector cells carrying an antigen receptor binding to the antigen or cells expressing the antigen. It is preferred that the vaccine antigen (similar to the disease-associated antigen) provides the relevant epitope for binding by the antigen binding domain present in the immune effector cells. In one embodiment, the vaccine antigen (similar to the disease-associated antigen) is expressed on the surface of a cell such as an antigen-presenting cell so as to provide the relevant epitope for binding by immune effector cells. In one embodiment, the vaccine antigen (similar to the disease-associated antigen) is expressed by and presented on the surface of a cell such as an antigen-presenting cell in the context of MHC so as to provide the relevant epitope for binding by immune effector cells. The vaccine antigen may be a recombinant antigen.

In one embodiment of all aspects of the invention, the nucleic acid encoding the vaccine antigen is expressed in cells of a subject to provide the antigen or a procession product thereof for binding by the antigen receptor expressed by immune effector cells, said binding resulting in stimulation, priming and/or expansion of the immune effector cells.

The term “immunologically equivalent” means that the immunologically equivalent molecule such as the immunologically equivalent amino acid sequence exhibits the same or essentially the same immunological properties and/or exerts the same or essentially the same immunological effects, e.g., with respect to the type of the immunological effect. In the context of the present disclosure, the term “immunologically equivalent” is preferably used with respect to the immunological effects or properties of antigens or antigen variants used for immunization. For example, an amino acid sequence is immunologically equivalent to a reference amino acid sequence if said amino acid sequence when exposed to the immune system of a subject such as T cells binding to the reference amino acid sequence or cells expressing the reference amino acid sequence induces an immune reaction having a specificity of reacting with the reference amino acid sequence. Thus, a molecule which is immunologically equivalent to an antigen exhibits the same or essentially the same properties and/or exerts the same or essentially the same effects regarding the stimulation, priming and/or expansion of T cells as the antigen to which the T cells are targeted.

“Activation” or “stimulation”, as used herein, refers to the state of an immune effector cell such as T cell that has been sufficiently stimulated to induce detectable cellular proliferation. Activation can also be associated with initiation of signaling pathways, induced cytokine production, and detectable effector functions. The term “activated immune effector cells” refers to, among other things, immune effector cells that are undergoing cell division.

The term “priming” refers to a process wherein an immune effector cell such as a T cell has its first contact with its specific antigen and causes differentiation into effector cells such as effector T cells.

The term “clonal expansion” or “expansion” refers to a process wherein a specific entity is multiplied. In the context of the present disclosure, the term is preferably used in the context of an immunological response in which lymphocytes are stimulated by an antigen, proliferate, and the specific lymphocyte recognizing said antigen is amplified. Preferably, clonal expansion leads to differentiation of the lymphocytes.

The term “antigen” relates to an agent comprising an epitope against which an immune response can be generated. The term “antigen” includes, in particular, proteins and peptides. In one embodiment, an antigen is presented or present on the surface of cells of the immune system such as antigen presenting cells like dendritic cells or macrophages. An antigen or a procession product thereof such as a T cell epitope is in one embodiment bound by an antigen receptor. Accordingly, an antigen or a procession product thereof may react specifically with immune effector cells such as T-lymphocytes (T cells). In one embodiment, an antigen is a disease-associated antigen, such as a tumor antigen, a viral antigen, or a bacterial antigen and an epitope is derived from such antigen.

The term “disease-associated antigen” is used in its broadest sense to refer to any antigen associated with a disease. A disease-associated antigen is a molecule which contains epitopes that will stimulate a host's immune system to make a cellular antigen-specific immune response and/or a humoral antibody response against the disease. The disease-associated antigen or an epitope thereof may therefore be used for therapeutic purposes. Disease-associated antigens may be associated with infection by microbes, typically microbial antigens, or associated with cancer, typically tumors.

The term “tumor antigen” or “tumor-associated antigen” refers to a constituent of cancer cells which may be derived from the cytoplasm, the cell surface and the cell nucleus. In particular, it refers to those antigens which are produced intracellularly or as surface antigens on tumor cells. A tumor antigen is typically expressed preferentially by cancer cells (e.g., it is expressed at higher levels in cancer cells than in non-cancer cells) and in some instances it is expressed solely by cancer cells. Examples of tumor antigens include, without limitation, p53, ART-4, BAGE, beta-catenin/m, Bcr-abL CAMEL, CAP-1, CASP-8, CDC27/m, CDK4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A 10, MAGE-A 11, or MAGE-A12, MAGE-B, MAGE-C, MART-1/Melan-A, MC1R, Myosin/m, MUC1, MUM-1, MUM-2, MUM-3, NA88-A, NF1, NY-ESO-1, NY-BR-1, pI90 minor BCR-abL, Pml/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE, WT, and WT-1. Particularly, preferred tumor antigens are proteins of the claudin family, such as CLAUDIN-6 or CLAUDIN-18.2.

The term “viral antigen” refers to any viral component having antigenic properties, i.e. being able to provoke an immune response in an individual. The viral antigen may be a viral ribonucleoprotein or an envelope protein.

The term “bacterial antigen” refers to any bacterial component having antigenic properties, i.e. being able to provoke an immune response in an individual. The bacterial antigen may be derived from the cell wall or cytoplasm membrane of the bacterium.

The term “expressed on the cell surface” or “associated with the cell surface” means that a molecule such as a receptor or antigen is associated with and located at the plasma membrane of a cell, wherein at least a part of the molecule faces the extracellular space of said cell and is accessible from the outside of said cell, e.g., by antibodies located outside the cell. In this context, a part is preferably at least 4, preferably at least 8, preferably at least 12, more preferably at least 20 amino acids. The association may be direct or indirect. For example, the association may be by one or more transmembrane domains, one or more lipid anchors, or by the interaction with any other protein, lipid, saccharide, or other structure that can be found on the outer leaflet of the plasma membrane of a cell. For example, a molecule associated with the surface of a cell may be a transmembrane protein having an extracellular portion or may be a protein associated with the surface of a cell by interacting with another protein that is a transmembrane protein.

“Cell surface” or “surface of a cell” is used in accordance with its normal meaning in the art, and thus includes the outside of the cell which is accessible to binding by proteins and other molecules. An antigen is expressed on the surface of cells if it is located at the surface of said cells and is accessible to binding by e.g. antigen-specific antibodies added to the cells. In one embodiment, an antigen expressed on the surface of cells is an integral membrane protein having an extracellular portion recognized by a CAR.

The term “extracellular portion” or “exodomain” in the context of the present invention refers to a part of a molecule such as a protein that is facing the extracellular space of a cell and preferably is accessible from the outside of said cell, e.g., by binding molecules such as antibodies located outside the cell. Preferably, the term refers to one or more extracellular loops or domains or a fragment thereof.

The term “epitope” refers to a part or fragment of a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, more preferably between about 8 and about 30, most preferably between about 10 and about 25 amino acids in length, for example, the epitope may be preferably 9, 10, 11, 12, 13, 14, 15, 16, 17, 18, 19, 20, 21, 22, 23, 24, or 25 amino acids in length. In one embodiment, an epitope is between about 10 and about 25 amino acids in length. The term “epitope” includes T cell epitopes.

The term “T cell epitope” refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

In one embodiment, the target antigen is a tumor antigen and the vaccine antigen or a fragment thereof (e.g., an epitope) is derived from the tumor antigen. The tumor antigen may be a “standard” antigen, which is generally known to be expressed in various cancers. The tumor antigen may also be a “neo-antigen”, which is specific to an individual's tumor and has not been previously recognized by the immune system. A neo-antigen or neo-epitope may result from one or more cancer-specific mutations in the genome of cancer cells resulting in amino acid changes. If the tumor antigen is a neo-antigen, the vaccine antigen preferably comprises an epitope or a fragment of said neo-antigen comprising one or more amino acid changes.

Cancer mutations vary with each individual. Thus, cancer mutations that encode novel epitopes (neo-epitopes) represent attractive targets in the development of vaccine compositions and immunotherapies. The efficacy of tumor immunotherapy relies on the selection of cancer-specific antigens and epitopes capable of inducing a potent immune response within a host. RNA can be used to deliver patient-specific tumor epitopes to a patient. Dendritic cells (DCs) residing in the spleen represent antigen-presenting cells of particular interest for RNA expression of immunogenic epitopes or antigens such as tumor epitopes. The use of multiple epitopes has been shown to promote therapeutic efficacy in tumor vaccine compositions. Rapid sequencing of the tumor mutanome may provide multiple epitopes for individualized vaccines which can be encoded by RNA described herein, e.g., as a single polypeptide wherein the epitopes are optionally separated by linkers. In certain embodiments of the present disclosure, the RNA encodes at least one epitope, at least two epitopes, at least three epitopes, at least four epitopes, at least five epitopes, at least six epitopes, at least seven epitopes, at least eight epitopes, at least nine epitopes, or at least ten epitopes. Exemplary embodiments include RNA that encodes at least five epitopes (termed a “pentatope”) and RNA that encodes at least ten epitopes (termed a “decatope”).

According to the various aspects of the invention, the aim is preferably to provide an immune response against cancer cells expressing a tumor antigen such as CLDN6 or CLDN18.2 and to treat a cancer disease involving cells expressing a tumor antigen such as CLDN6 or CLDN18.2. Preferably the invention involves the administration of antigen receptor-engineered immune effector cells such as T cells targeted against cancer cells expressing a tumor antigen such as CLDN6 or CLDN18.2.

The peptide and protein antigen can be 2-100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide can be greater than 50 amino acids. In some embodiments, the peptide can be greater than 100 amino acids.

According to the invention, the vaccine antigen should be recognizable by an immune effector cell. Preferably, the antigen if recognized by an immune effector cell is able to induce in the presence of appropriate co-stimulatory signals, stimulation, priming and/or expansion of the immune effector cell carrying an antigen receptor recognizing the antigen. In the context of the embodiments of the present invention, the antigen is preferably present on the surface of a cell, preferably an antigen presenting cell. Recognition of the antigen on the surface of a diseased cell may result in an immune reaction against the antigen (or cell expressing the antigen).

In one embodiment of all aspects of the invention, an antigen is expressed in a diseased cell such as a cancer cell. In one embodiment, an antigen is expressed on the surface of a diseased cell such as a cancer cell. In one embodiment, an antigen receptor is a CAR which binds to an extracellular domain or to an epitope in an extracellular domain of an antigen. In one embodiment, a CAR binds to native epitopes of an antigen present on the surface of living cells. In one embodiment, binding of a CAR when expressed by T cells and/or present on T cells to an antigen present on cells such as antigen presenting cells results in stimulation, priming and/or expansion of said T cells. In one embodiment, binding of a CAR when expressed by T cells and/or present on T cells to an antigen present on diseased cells such as cancer cells results in cytolysis and/or apoptosis of the diseased cells, wherein said T cells preferably release cytotoxic factors, e.g. perforins and granzymes.

Chemotherapy

In certain embodiments, additional treatments may be administered to a patient in combination with the treatments described herein. Such additional treatments includes classical cancer therapy, e.g., radiation therapy, surgery, hyperthermia therapy and/or chemotherapy.

Chemotherapy is a type of cancer treatment that uses one or more anti-cancer drugs (chemotherapeutic agents), usually as part of a standardized chemotherapy regimen. The term chemotherapy has come to connote non-specific usage of intracellular poisons to inhibit mitosis. The connotation excludes more selective agents that block extracellular signals (signal transduction). The development of therapies with specific molecular or genetic targets, which inhibit growth-promoting signals from classic endocrine hormones (primarily estrogens for breast cancer and androgens for prostate cancer) are now called hormonal therapies. By contrast, other inhibitions of growth-signals like those associated with receptor tyrosine kinases are referred to as targeted therapy.

Importantly, the use of drugs (whether chemotherapy, hormonal therapy or targeted therapy) constitutes systemic therapy for cancer in that they are introduced into the blood stream and are therefore in principle able to address cancer at any anatomic location in the body. Systemic therapy is often used in conjunction with other modalities that constitute local therapy (i.e. treatments whose efficacy is confined to the anatomic area where they are applied) for cancer such as radiation therapy, surgery or hyperthermia therapy.

Traditional chemotherapeutic agents are cytotoxic by means of interfering with cell division (mitosis) but cancer cells vary widely in their susceptibility to these agents. To a large extent, chemotherapy can be thought of as a way to damage or stress cells, which may then lead to cell death if apoptosis is initiated.

Chemotherapeutic agents include alkylating agents, antimetabolites, anti-microtubule agents, topoisomerase inhibitors, and cytotoxic antibiotics.

Alkylating agents have the ability to alkylate many molecules, including proteins, RNA and DNA. The subtypes of alkylating agents are the nitrogen mustards, nitrosoureas, tetrazines, aziridines, cisplatins and derivatives, and non-classical alkylating agents. Nitrogen mustards include mechlorethamine, cyclophosphamide, melphalan, chlorambucil, ifosfamide and busulfan. Nitrosoureas include N-Nitroso-N-methylurea (MNU), carmustine (BCNU), lomustine (CCNU) and semustine (MeCCNU), fotemustine and streptozotocin. Tetrazines include dacarbazine, mitozolomide and temozolomide. Aziridines include thiotepa, mytomycin and diaziquone (AZQ). Cisplatin and derivatives include cisplatin, carboplatin and oxaliplatin. They impair cell function by forming covalent bonds with the amino, carboxyl, sulfhydryl, and phosphate groups in biologically important molecules. Non-classical alkylating agents include procarbazine and hexamethylmelamine. In one particularly preferred embodiment, the alkylating agent is cyclophosphamide.

Anti-metabolites are a group of molecules that impede DNA and RNA synthesis. Many of them have a similar structure to the building blocks of DNA and RNA. Anti-metabolites resemble either nucleobases or nucleosides, but have altered chemical groups. These drugs exert their effect by either blocking the enzymes required for DNA synthesis or becoming incorporated into DNA or RNA. Subtypes of the anti-metabolites are the anti-folates, fluoropyrimidines, deoxynucleoside analogues and thiopurines. The anti-folates include methotrexate and pemetrexed. The fluoropyrimidines include fluorouracil and capecitabine. The deoxynucleoside analogues include cytarabine, gemcitabine, decitabine, azacitidine, fludarabine, nelarabine, cladribine, clofarabine, and pentostatin. The thiopurines include thioguanine and mercaptopurine.

Anti-microtubule agents block cell division by preventing microtubule function. The vinca alkaloids prevent the formation of the microtubules, whereas the taxanes prevent the microtubule disassembly. Vinca alkaloids include vinorelbine, vindesine, and vinflunine. Taxanes include docetaxel (Taxotere) and paclitaxel (Taxol).

Topoisomerase inhibitors are drugs that affect the activity of two enzymes: topoisomerase I and topoisomerase II and include irinotecan, topotecan, camptothecin, etoposide, doxorubicin, mitoxantrone, teniposide, novobiocin, merbarone, and aclarubicin.

The cytotoxic antibiotics are a varied group of drugs that have various mechanisms of action. The common theme that they share in their chemotherapy indication is that they interrupt cell division. The most important subgroup is the anthracyclines (e.g., doxorubicin, daunorubicin, epirubicin, idarubicin pirarubicin, and aclarubicin) and the bleomycins; other prominent examples include mitomycin C, mitoxantrone, and actinomycin.

In one embodiment, prior to administration of immune effector cells, a lymphodepleting treatment may be applied, e.g., by administering cyclophosphamide and fludarabine. Such treatment may increase cell persistence and the incidence and duration of clinical responses.

Immune Checkpoint Inhibitors

In certain embodiments, immune checkpoint inhibitors are used in combination with other therapeutic agents described herein.

As used herein, “immune checkpoint” refers to co-stimulatory and inhibitory signals that regulate the amplitude and quality of T cell receptor recognition of an antigen. In certain embodiments, the immune checkpoint is an inhibitory signal. In certain embodiments, the inhibitory signal is the interaction between PD-1 and PD-L1. In certain embodiments, the inhibitory signal is the interaction between CTLA-4 and CD80 or CD86 to displace CD28 binding. In certain embodiments the inhibitory signal is the interaction between LAG3 and MHC class II molecules. In certain embodiments, the inhibitory signal is the interaction between TIM3 and galectin 9.

As used herein, “immune checkpoint inhibitor” refers to a molecule that totally or partially reduces, inhibits, interferes with or modulates one or more checkpoint proteins. In certain embodiments, the immune checkpoint inhibitor prevents inhibitory signals associated with the immune checkpoint. In certain embodiments, the immune checkpoint inhibitor is an antibody, or fragment thereof that disrupts inhibitory signaling associated with the immune checkpoint. In certain embodiments, the immune checkpoint inhibitor is a small molecule that disrupts inhibitory signaling. In certain embodiments, the immune checkpoint inhibitor is an antibody, fragment thereof, or antibody mimic, that prevents the interaction between checkpoint blocker proteins, e.g., an antibody, or fragment thereof, that prevents the interaction between PD-1 and PD-L1. In certain embodiments, the immune checkpoint inhibitor is an antibody, or fragment thereof, that prevents the interaction between CTLA-4 and CD80 or CD86. In certain embodiments, the immune checkpoint inhibitor is an antibody, or fragment thereof, that prevents the interaction between LAG3 and its ligands, or TIM-3 and its ligands. The checkpoint inhibitor may also be in the form of the soluble form of the molecules (or variants thereof) themselves, e.g., a soluble PD-L1 or PD-L1 fusion.

The “Programmed Death-1 (PD-1)” receptor refers to an immuno-inhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1.

“Programmed Death Ligand-1 (PD-L1)” is one of two cell surface glycoprotein ligands for PD-1 (the other being PD-L2) that downregulates T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1.

“Cytotoxic T Lymphocyte Associated Antigen-4 (CTLA-4)” is a T cell surface molecule and is a member of the immunoglobulin superfamily. This protein downregulates the immune system by binding to CD80 and CD86. The term “CTLA-4” as used herein includes human CTLA-4 (hCTLA-4), variants, isoforms, and species homologs of hCTLA-4, and analogs having at least one common epitope with hCTLA-4.

“Lymphocyte Activation Gene-3 (LAG3)” is an inhibitory receptor associated with inhibition of lymphocyte activity by binding to MHC class II molecules. This receptor enhances the function of Treg cells and inhibits CD8⁺ effector T cell function. The term “LAG3” as used herein includes human LAG3 (hLAG3), variants, isoforms, and species homologs of hLAG3, and analogs having at least one common epitope.

“T Cell Membrane Protein-3 (TIM3)” is an inhibitory receptor involved in the inhibition of lymphocyte activity by inhibition of TH1 cell responses. Its ligand is galectin 9, which is upregulated in various types of cancers. The term “TIM3” as used herein includes human TIM3 (hTIM3), variants, isoforms, and species homologs of hTIM3, and analogs having at least one common epitope.

The “B7 family” refers to inhibitory ligands with undefined receptors. The B7 family encompasses B7-H3 and B7-H4, both upregulated on tumor cells and tumor infiltrating cells.

In certain embodiments, the immune checkpoint inhibitor suitable for use in the methods disclosed herein, is an antagonist of inhibitory signals, e.g., an antibody which targets, for example, PD-1, PD-L1, CTLA-4, LAG3, B7-H3, B7-H4, or TIM3. These ligands and receptors are reviewed in Pardoll, D., Nature. 12: 252-264, 2012.

In certain embodiments, the immune checkpoint inhibitor is an antibody or an antigen-binding portion thereof, that disrupts or inhibits signaling from an inhibitory immunoregulator. In certain embodiments, the immune checkpoint inhibitor is a small molecule that disrupts or inhibits signaling from an inhibitory immunoregulator.

In certain embodiments, the inhibitory immunoregulator is a component of the PD-1/PD-L1 signaling pathway. Accordingly, certain embodiments of the disclosure provide for administering to a subject an antibody or an antigen-binding portion thereof that disrupts the interaction between the PD-1 receptor and its ligand, PD-L1. Antibodies which bind to PD-1 and disrupt the interaction between the PD-1 and its ligand, PD-L1, are known in the art. In certain embodiments, the antibody or antigen-binding portion thereof binds specifically to PD-1. In certain embodiments, the antibody or antigen-binding portion thereof binds specifically to PD-L1 and inhibits its interaction with PD-1, thereby increasing immune activity.

In certain embodiments, the inhibitory immunoregulator is a component of the CTLA4 signaling pathway. Accordingly, certain embodiments of the disclosure provide for administering to a subject an antibody or an antigen-binding portion thereof that targets CTLA4 and disrupts its interaction with CD80 and CD86.

In certain embodiments, the inhibitory immunoregulator is a component of the LAG3 (lymphocyte activation gene 3) signaling pathway. Accordingly, certain embodiments of the disclosure provide for administering to a subject an antibody or an antigen-binding portion thereof that targets LAG3 and disrupts its interaction with MHC class II molecules.

In certain embodiments, the inhibitory immunoregulator is a component of the B7 family signaling pathway. In certain embodiments, the B7 family members are B7-H3 and B7-H4. Accordingly, certain embodiments of the disclosure provide for administering to a subject an antibody or an antigen-binding portion thereof that targets B7-H3 or H4. The B7 family does not have any defined receptors but these ligands are upregulated on tumor cells or tumor-infiltrating cells. Preclinical mouse models have shown that blockade of these ligands can enhance anti-tumor immunity.

In certain embodiments, the inhibitory immunoregulator is a component of the TIM3 (T cell membrane protein 3) signaling pathway. Accordingly, certain embodiments of the disclosure provide for administering to a subject an antibody or an antigen-binding portion thereof that targets TIM3 and disrupts its interaction with galectin 9.

It will be understood by one of ordinary skill in the art that other immune checkpoint targets can also be targeted by antagonists or antibodies, provided that the targeting results in the stimulation of an immune response such as an anti-tumor immune response as reflected in, e.g., an increase in T cell proliferation, enhanced T cell activation, and/or increased cytokine production (e.g., IFN-γ, IL2).

RNA Targeting

It is particularly preferred according to the invention that the peptides, proteins or polypeptides described herein, in particular the vaccine antigens, are administered in the form of RNA encoding the peptides, proteins or polypeptides described herein. In one embodiment, different peptides, proteins or polypeptides described herein are encoded by different RNA molecules.

In one embodiment, the RNA is formulated in a delivery vehicle. In one embodiment, the delivery vehicle comprises particles. In one embodiment, the delivery vehicle comprises at least one lipid. In one embodiment, the at least one lipid comprises at least one cationic lipid. In one embodiment, the lipid forms a complex with and/or encapsulates the RNA. In one embodiment, the lipid is comprised in a vesicle encapsulating the RNA. In one embodiment, the RNA is formulated in liposomes.

According to the disclosure, after administration of the RNA described herein, at least a portion of the RNA is delivered to a target cell. In one embodiment, at least a portion of the RNA is delivered to the cytosol of the target cell. In one embodiment, the RNA is translated by the target cell to produce the encoded peptide or protein.

Some aspects of the disclosure involve the targeted delivery of the RNA disclosed herein (e.g., RNA encoding vaccine antigen).

In one embodiment, the disclosure involves targeting the lymphatic system, in particular secondary lymphoid organs, more specifically spleen. Targeting the lymphatic system, in particular secondary lymphoid organs, more specifically spleen is in particular preferred if the RNA administered is RNA encoding vaccine antigen.

In one embodiment, the target cell is a spleen cell. In one embodiment, the target cell is an antigen presenting cell such as a professional antigen presenting cell in the spleen. In one embodiment, the target cell is a dendritic cell in the spleen.

The “lymphatic system” is part of the circulatory system and an important part of the immune system, comprising a network of lymphatic vessels that carry lymph. The lymphatic system consists of lymphatic organs, a conducting network of lymphatic vessels, and the circulating lymph. The primary or central lymphoid organs generate lymphocytes from immature progenitor cells. The thymus and the bone marrow constitute the primary lymphoid organs. Secondary or peripheral lymphoid organs, which include lymph nodes and the spleen, maintain mature naïve lymphocytes and initiate an adaptive immune response.

RNA may be delivered to spleen by so-called lipoplex formulations, in which the RNA is bound to liposomes comprising a cationic lipid and optionally an additional or helper lipid to form injectable nanoparticle formulations. The liposomes may be obtained by injecting a solution of the lipids in ethanol into water or a suitable aqueous phase. RNA lipoplex particles may be prepared by mixing the liposomes with RNA. Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages.

In the context of the present disclosure, the term “RNA lipoplex particle” relates to a particle that contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE. In one embodiment, a RNA lipoplex particle is a nanoparticle.

As used herein, a “cationic lipid” refers to a lipid having a net positive charge. Cationic lipids bind negatively charged RNA by electrostatic interaction to the lipid matrix. Generally, cationic lipids possess a lipophilic moiety, such as a sterol, an acyl or diacyl chain, and the head group of the lipid typically carries the positive charge. Examples of cationic lipids include, but are not limited to 1,2-di-O-octadecenyl-3-trimethylammonium propane (DOTMA), dimethyldioctadecylammonium (DDAB); 1,2-dioleoyl-3-trimethylammonium propane (DOTAP); 1,2-dioleoyl-3-dimethylammonium-propane (DODAP); 1,2-diacyloxy-3-dimethylammonium propanes; 1,2-dialkyloxy-3- dimethylammonium propanes; dioctadecyldimethyl ammonium chloride (DODAC), 2,3-di(tetradecoxy)propyl-(2-hydroxyethyl)-dimethylazanium (DMRIE), 1,2-dimyristoyl-sn-glycero-3-ethylphosphocholine (DMEPC), I,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA). Preferred are DOTMA, DOTAP, DODAC, and DOSPA. In specific embodiments, the cationic lipid is DOTMA and/or DOTAP.

An additional lipid may be incorporated to adjust the overall positive to negative charge ratio and physical stability of the RNA lipoplex particles. In certain embodiments, the additional lipid is a neutral lipid. As used herein, a “neutral lipid” refers to a lipid having a net charge of zero. Examples of neutral lipids include, but are not limited to, 1,2-di-(9Z-octadecenoyl)-sn-glycero-3-phosphoethanolamine (DOPE), 1,2-dioleoyl-sn-glycero-3-phosphocholine (DOPC), diacylphosphatidyl choline, diacylphosphatidyl ethanol amine, ceramide, sphingoemyelin, cephalin, cholesterol, and cerebroside. In specific embodiments, the additional lipid is DOPE, cholesterol and/or DOPC.

In certain embodiments, the RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

RNA lipoplex particles described herein have an average diameter that in one embodiment ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

The electric charge of the RNA lipoplex particles of the present disclosure is the sum of the electric charges present in the at least one cationic lipid and the electric charges present in the RNA. The charge ratio is the ratio of the positive charges present in the at least one cationic lipid to the negative charges present in the RNA. The charge ratio of the positive charges present in the at least one cationic lipid to the negative charges present in the RNA is calculated by the following equation: charge ratio=[(cationic lipid concentration (mol))*(the total number of positive charges in the cationic lipid)]/[(RNA concentration (mol))*(the total number of negative charges in RNA)].

The spleen targeting RNA lipoplex particles described herein at physiological pH preferably have a net negative charge such as a charge ratio of positive charges to negative charges from about 1.9:2 to about 1:2. In specific embodiments, the charge ratio of positive charges to negative charges in the RNA lipoplex particles at physiological pH is about 1.9:2.0, about 1.8:2.0, about 1.7:2.0, about 1.6:2.0, about 1.5:2.0, about 1.4:2.0, about 1.3:2.0, about 1.2:2.0, about 1.1:2.0, or about 1:2.0.

RNA delivery systems have an inherent preference to the liver. This pertains to lipid-based particles, cationic and neutral nanoparticles, in particular lipid nanoparticles such as liposomes, nanomicelles and lipophilic ligands in bioconjugates. Liver accumulation is caused by the discontinuous nature of the hepatic vasculature or the lipid metabolism (liposomes and lipid or cholesterol conjugates).

In one embodiment of the targeted delivery of a cytokine such as IL2, the target organ is liver and the target tissue is liver tissue. The delivery to such target tissue is preferred, in particular, if presence of the cytokine in this organ or tissue is desired and/or if it is desired to express large amounts of the cytokine and/or if systemic presence of the cytokine, in particular in significant amounts, is desired or required.

In one embodiment, RNA encoding a cytokine is administered in a formulation for targeting liver. Such formulations are described herein above.

For in vivo delivery of RNA to the liver, a drug delivery system may be used to transport the RNA into the liver by preventing its degradation. For example, polyplex nanomicelles consisting of a poly(ethylene glycol) (PEG)-coated surface and an mRNA-containing core is a useful system because the nanomicelles provide excellent in vivo stability of the RNA, under physiological conditions. Furthermore, the stealth property provided by the polyplex nanomicelle surface, composed of dense PEG palisades, effectively evades host immune defenses.

Pharmaceutical Compositions

The peptides, proteins, polypeptides, RNA, RNA particles, immune effector cells and further agents, e.g., immune checkpoint inhibitors, described herein may be administered in pharmaceutical compositions or medicaments for therapeutic or prophylactic treatments and may be administered in the form of any suitable pharmaceutical composition which may comprise a pharmaceutically acceptable carrier and may optionally comprise one or more adjuvants, stabilizers etc. In one embodiment, the pharmaceutical composition is for therapeutic or prophylactic treatments, e.g., for use in treating or preventing a disease involving an antigen such as a cancer disease such as those described herein.

The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation. In the context of the present disclosure, the pharmaceutical composition comprises peptides, proteins, polypeptides, RNA, RNA particles, immune effector cells and/or further agents as described herein.

The pharmaceutical compositions of the present disclosure may comprise one or more adjuvants or may be administered with one or more adjuvants. The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cytokines, such as monokines, lymphokines, interleukins, chemokines. The cytokines may be IL1, IL2, IL3, IL4, IL5, IL6, IL7, IL8, IL9, IL10, IL12, IFNα, IFNγ, GM-CSF, LT-a. Further known adjuvants are aluminium hydroxide, Freund's adjuvant or oil such as Montanide® ISA51. Other suitable adjuvants for use in the present disclosure include lipopeptides, such as Pam3Cys.

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” or “therapeutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

The term “sub-therapeutic amount” typically refers to a less than standard therapeutic amount of a pharmaceutical agent, meaning that the amount required for the desired effect is lower than when the pharmaceutical agent is used alone. As used herein, the term “sub-therapeutic amount” means that the dosage or amount of a particular pharmaceutical agent is insufficient to achieve the desired pharmacological action in the absence of other compounds, drugs or pharmaceutical agents, e.g., in the absence of vaccine antigen. Such desired pharmacological action may include the complete or essentially complete rejection of solid tumors. Subtherapeutic amounts and doses will usually not be less than about 5%, typically not less than about 10%, and typically not greater than about 75%, more typically not greater than about 60%, of the therapeutic dosage or amount. Normally, the number of immune effector cells administered (including in vivo generation in a subject) for CAR T cell therapy of human beings is about 10⁹ per dose (equivalent to 1.33×10⁷ per kg) or higher (equivalent to 1.3×10⁷ per kg). Furthermore, some therapeutic approaches comprise repetitive administration of CAR T cells in a short time period (e.g. less than 4 weeks) to improve safety by dose escalation and/or to maintain the number of effective T cells in the patient. This leads to even higher “accumulated doses” within such time periods. Thus, a “sub-therapeutic amount” of immune effector cells genetically modified to express an antigen receptor is an amount of such cells per initial dose and/or accumulated dose over a time period of at least 2 days, at least 3 days, at least 4 days, at least 5 days, at least 6 days, at least 7 days, at least 14 days, at least 21 days, at least 28 days or even longer of 10⁸ or less, 10⁷ or less, 10⁶ or less, 10⁵ or less, 10⁴ or less, 10³ or less or even lower. In one embodiment, a “sub-therapeutic amount” of immune effector cells genetically modified to express an antigen receptor relates to a single dose of such cells in an amount of 10⁸ or less, 10⁷ or less, 10⁶ or less, 10⁵ or less, 10⁴ or less, 10³ or less or even lower. The term “single dose” means that one dose of a therapeutic substance is administered for a prolonged time. The term “prolonged time” comprises a period of at least 14 days, at least 21 days, at least 28 days, at least 3 months, at least 6 months or even longer.

The pharmaceutical compositions of the present disclosure may contain salts, buffers, preservatives, and optionally other therapeutic agents. In one embodiment, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In one embodiment, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

In one embodiment, pharmaceutical compositions described herein may be administered intravenously, intraarterially, subcutaneously, intradermally or intramuscularly. In certain embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the pharmaceutical compositions is formulated for systemic administration. In another preferred embodiment, the systemic administration is by intravenous administration. In one embodiment of all aspects of the invention, RNA encoding an antigen is administered systemically.

The term “co-administering” as used herein means a process whereby different compounds or compositions (e.g., immune effector cells (which may be “administered” by in vivo generation in a subject), and antigen, polynucleotide encoding antigen, or host cell genetically modified to express antigen) are administered to the same patient. The different compounds or compositions may be administered simultaneously, at essentially the same time, or sequentially. The antigen, polynucleotide encoding antigen, or host cell genetically modified to express antigen in one embodiment is administered following administration or generation of immune effector cells genetically modified to express an antigen receptor, e.g., at least one day, such as 1 to 10 days or 1 to 5 days following administration or generation of immune effector cells genetically modified to express an antigen receptor. The antigen, polynucleotide encoding antigen, or host cell genetically modified to express antigen may be administered several times over time in constant or different time intervals, e.g., following administration or generation of immune effector cells genetically modified to express an antigen receptor, e.g., in time intervals of between 10 and 40 days, wherein the first administration of antigen, polynucleotide encoding antigen, or host cell genetically modified to express antigen may be at least one day, such as 1 to 10 days or 1 to 5 days following administration or generation of immune effector cells genetically modified to express an antigen receptor.

Treatments

The agents, compositions and methods described herein can be used to treat a subject with a disease, e.g., a disease characterized by the presence of diseased cells expressing an antigen. Particularly preferred diseases are cancer diseases. For example, if the antigen is derived from a virus, the agents, compositions and methods may be useful in the treatment of a viral disease caused by said virus. If the antigen is a tumor antigen, the agents, compositions and methods may be useful in the treatment of a cancer disease wherein cancer cells express said tumor antigen.

The agents, compositions and methods described herein may be used in the therapeutic or prophylactic treatment of various diseases, wherein provision of immune effector cells and/or activity of immune effector cells as described herein is beneficial for a patient such as cancer and infectious diseases In one embodiment, the agents, compositions and methods described herein are useful in a prophylactic and/or therapeutic treatment of a disease involving an antigen.

The term “disease” refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g. mouse, rat, rabbit, dog, cat, cattle, swine, sheep, horse or primate) that can be afflicted with or is susceptible to a disease or disorder (e.g., cancer) but may or may not have the disease or disorder. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

In one embodiment of the disclosure, the aim is to provide an immune response against diseased cells expressing an antigen such as cancer cells expressing a tumor antigen, and to treat a disease such as a cancer disease involving cells expressing an antigen such as a tumor antigen.

An immune response against an antigen may be elicited which may be therapeutic or partially or fully protective. Pharmaceutical compositions described herein are applicable for inducing or enhancing an immune response. Pharmaceutical compositions described herein are thus useful in a prophylactic and/or therapeutic treatment of a disease involving an antigen.

As used herein, “immune response” refers to an integrated bodily response to an antigen or a cell expressing an antigen and refers to a cellular immune response and/or a humoral immune response.

“Cell-mediated immunity”, “cellular immunity”, “cellular immune response”, or similar terms are meant to include a cellular response directed to cells characterized by expression of an antigen, in particular characterized by presentation of an antigen with class I or class II MHC. The cellular response relates to cells called T cells or T lymphocytes which act as either “helpers” or “killers”. The helper T cells (also termed CD4⁺ T cells) play a central role by regulating the immune response and the killer cells (also termed cytotoxic T cells, cytolytic T cells, CD8⁺ T cells or CTLs) kill diseased cells such as cancer cells, preventing the production of more diseased cells.

The present disclosure contemplates an immune response that may be protective, preventive, prophylactic and/or therapeutic. As used herein, “induces [or inducing] an immune response” may indicate that no immune response against a particular antigen was present before induction or it may indicate that there was a basal level of immune response against a particular antigen before induction, which was enhanced after induction. Therefore, “induces [or inducing] an immune response” includes “enhances [or enhancing] an immune response”.

The term “immunotherapy” relates to the treatment of a disease or condition by inducing, or enhancing an immune response. The term “immunotherapy” includes antigen immunization or antigen vaccination.

The terms “immunization” or “vaccination” describe the process of administering an antigen to an individual with the purpose of inducing an immune response, for example, for therapeutic or prophylactic reasons.

The term “macrophage” refers to a subgroup of phagocytic cells produced by the differentiation of monocytes. Macrophages which are activated by inflammation, immune cytokines or microbial products nonspecifically engulf and kill foreign pathogens within the macrophage by hydrolytic and oxidative attack resulting in degradation of the pathogen. Peptides from degraded proteins are displayed on the macrophage cell surface where they can be recognized by T cells, and they can directly interact with antibodies on the B cell surface, resulting in T and B cell activation and further stimulation of the immune response. Macrophages belong to the class of antigen presenting cells. In one embodiment, the macrophages are splenic macrophages.

The term “dendritic cell” (DC) refers to another subtype of phagocytic cells belonging to the class of antigen presenting cells. In one embodiment, dendritic cells are derived from hematopoietic bone marrow progenitor cells. These progenitor cells initially transform into immature dendritic cells. These immature cells are characterized by high phagocytic activity and low T cell activation potential. Immature dendritic cells constantly sample the surrounding environment for pathogens such as viruses and bacteria. Once they have come into contact with a presentable antigen, they become activated into mature dendritic cells and begin to migrate to the spleen or to the lymph node. Immature dendritic cells phagocytose pathogens and degrade their proteins into small pieces and upon maturation present those fragments at their cell surface using MHC molecules. Simultaneously, they upregulate cell-surface receptors that act as co-receptors in T cell activation such as CD80, CD86, and CD40 greatly enhancing their ability to activate T cells. They also upregulate CCR7, a chemotactic receptor that induces the dendritic cell to travel through the blood stream to the spleen or through the lymphatic system to a lymph node. Here they act as antigen-presenting cells and activate helper T cells and killer T cells as well as B cells by presenting them antigens, alongside non-antigen specific co-stimulatory signals. Thus, dendritic cells can actively induce a T cell- or B cell-related immune response. In one embodiment, the dendritic cells are splenic dendritic cells.

The term “antigen presenting cell” (APC) is a cell of a variety of cells capable of displaying, acquiring, and/or presenting at least one antigen or antigenic fragment on (or at) its cell surface. Antigen-presenting cells can be distinguished in professional antigen presenting cells and non-professional antigen presenting cells.

The term “professional antigen presenting cells” relates to antigen presenting cells which constitutively express the Major Histocompatibility Complex class II (MHC class II) molecules required for interaction with naive T cells. If a T cell interacts with the MHC class II molecule complex on the membrane of the antigen presenting cell, the antigen presenting cell produces a co-stimulatory molecule inducing activation of the T cell. Professional antigen presenting cells comprise dendritic cells and macrophages.

The term “non-professional antigen presenting cells” relates to antigen presenting cells which do not constitutively express MHC class II molecules, but upon stimulation by certain cytokines such as interferon-gamma. Exemplary, non-professional antigen presenting cells include fibroblasts, thymic epithelial cells, thyroid epithelial cells, glial cells, pancreatic beta cells or vascular endothelial cells.

“Antigen processing” refers to the degradation of an antigen into procession products, which are fragments of said antigen (e.g., the degradation of a protein into peptides) and the association of one or more of these fragments (e.g., via binding) with MHC molecules for presentation by cells, such as antigen presenting cells to specific T cells.

The term “disease involving an antigen”, “disease involving cells expressing an antigen” or similar terms refer to any disease which implicates an antigen, e.g. a disease which is characterized by the presence of an antigen. The disease involving an antigen can be an infectious disease, or a cancer disease or simply cancer. As mentioned above, the antigen may be a disease-associated antigen, such as a tumor-associated antigen, a viral antigen, or a bacterial antigen. In one embodiment, a disease involving an antigen is a disease involving cells expressing an antigen, preferably on the cell surface.

The term “infectious disease” refers to any disease which can be transmitted from individual to individual or from organism to organism, and is caused by a microbial agent (e.g. common cold). Infectious diseases are known in the art and include, for example, a viral disease, a bacterial disease, or a parasitic disease, which diseases are caused by a virus, a bacterium, and a parasite, respectively. In this regard, the infectious disease can be, for example, hepatitis, sexually transmitted diseases (e.g. chlamydia or gonorrhea), tuberculosis, HIV/acquired immune deficiency syndrome (AIDS), diphtheria, hepatitis B, hepatitis C, cholera, severe acute respiratory syndrome (SARS), the bird flu, and influenza.

The terms “cancer disease” or “cancer” refer to or describe the physiological condition in an individual that is typically characterized by unregulated cell growth. Examples of cancers include, but are not limited to, carcinoma, lymphoma, blastoma, sarcoma, and leukemia. More particularly, examples of such cancers include bone cancer, blood cancer, lung cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma. The term “cancer” according to the disclosure also comprises cancer metastases.

The term “solid tumor” or “solid cancer” as used herein refers to the manifestation of a cancerous mass, as is well known in the art for example in Harrison's Principles of Internal Medicine, 14th edition. Preferably, the term refers to a cancer or carcinoma of body tissues other than blood, preferably other than blood, bone marrow, and lymphoid system. For example, but not by way of limitation, solid tumors include cancers of the prostate, lung cancer, colorectal tissue, bladder, oropharyngeal/laryngeal tissue, kidney, breast, endometrium, ovary, cervix, stomach, pancrease, brain, and central nervous system.

Combination strategies in cancer treatment may be desirable due to a resulting synergistic effect, which may be considerably stronger than the impact of a monotherapeutic approach. In one embodiment, the pharmaceutical composition is administered with an immunotherapeutic agent. As used herein “immunotherapeutic agent” relates to any agent that may be involved in activating a specific immune response and/or immune effector function(s). The present disclosure contemplates the use of an antibody as an immunotherapeutic agent. Without wishing to be bound by theory, antibodies are capable of achieving a therapeutic effect against cancer cells through various mechanisms, including inducing apoptosis, block components of signal transduction pathways or inhibiting proliferation of tumor cells. In certain embodiments, the antibody is a monoclonal antibody. A monoclonal antibody may induce cell death via antibody-dependent cell mediated cytotoxicity (ADCC), or bind complement proteins, leading to direct cell toxicity, known as complement dependent cytotoxicity (CDC). Non-limiting examples of anti-cancer antibodies and potential antibody targets (in brackets) which may be used in combination with the present disclosure include: Abagovomab (CA-125), Abciximab (CD41), Adecatumumab (EpCAM), Afutuzumab (CD20), Alacizumab pegol (VEGFR2), Altumomab pentetate (CEA), Amatuximab (MORAb-009), Anatumomab mafenatox (TAG-72), Apolizumab (HLA-DR), Arcitumomab (CEA), Atezolizumab (PD-L1), Bavituximab (phosphatidylserine), Bectumomab (CD22), Belimumab (BAFF), Bevacizumab (VEGF-A), Bivatuzumab mertansine (CD44 v6), Blinatumomab (CD 19), Brentuximab vedotin (CD30 TNFRSF8), Cantuzumab mertansin (mucin CanAg), Cantuzumab ravtansine (MUC1), Capromab pendetide (prostatic carcinoma cells), Carlumab (CNT0888), Catumaxomab (EpCAM, CD3), Cetuximab (EGFR), Citatuzumab bogatox (EpCAM), Cixutumumab (IGF-1 receptor), Claudiximab (Claudin), Clivatuzumab tetraxetan (MUC1), Conatumumab (TRAIL-R2), Dacetuzumab (CD40), Dalotuzumab (insulin-like growth factor I receptor), Denosumab (RANKL), Detumomab (B-lymphoma cell), Drozitumab (DR5), Ecromeximab (GD3 ganglioside), Edrecolomab (EpCAM), Elotuzumab (SLAMF7), Enavatuzumab (PDL192), Ensituximab (NPC-1C), Epratuzumab (CD22), Ertumaxomab (HER2/neu, CD3), Etaracizumab (integrin αvβ3), Farletuzumab (folate receptor 1), FBTA05 (CD20), Ficlatuzumab (SCH 900105), Figitumumab (IGF-1 receptor), Flanvotumab (glycoprotein 75), Fresolimumab (TGF-β), Galiximab (CD80), Ganitumab (IGF-I), Gemtuzumab ozogamicin (CD33), Gevokizumab (ILIIβ), Girentuximab (carbonic anhydrase 9 (CA-IX)), Glembatumumab vedotin (GPNMB), Ibritumomab tiuxetan (CD20), Icrucumab (VEGFR-1), Igovoma (CA-125), Indatuximab ravtansine (SDC1), Intetumumab (CD51), Inotuzumab ozogamicin (CD22), Ipilimumab (CD 152), Iratumumab (CD30), Labetuzumab (CEA), Lexatumumab (TRAIL-R2), Libivirumab (hepatitis B surface antigen), Lintuzumab (CD33), Lorvotuzumab mertansine (CD56), Lucatumumab (CD40), Lumiliximab (CD23), Mapatumumab (TRAIL-R1), Matuzumab (EGFR), Mepolizumab (IL5), Milatuzumab (CD74), Mitumomab (GD3 ganglioside), Mogamulizumab (CCR4), Moxetumomab pasudotox (CD22), Nacolomab tafenatox (C242 antigen), Naptumomab estafenatox (5T4), Namatumab (RON), Necitumumab (EGFR), Nimotuzumab (EGFR), Nivolumab (IgG4), Ofatumumab (CD20), Olaratumab (PDGF-R a), Onartuzumab (human scatter factor receptor kinase), Oportuzumab monatox (EpCAM), Oregovomab (CA-125), Oxelumab (OX-40), Panitumumab (EGFR), Patritumab (HER3), Pemtumoma (MUC1), Pertuzuma (HER2/neu), Pintumomab (adenocarcinoma antigen), Pritumumab (vimentin), Racotumomab (N-glycolylneuraminic acid), Radretumab (fibronectin extra domain-B), Rafivirumab (rabies virus glycoprotein), Ramucirumab (VEGFR2), Rilotumumab (HGF), Rituximab (CD20), Robatumumab (IGF-1 receptor), Samalizumab (CD200), Sibrotuzumab (FAP), Siltuximab (IL6), Tabalumab (BAFF), Tacatuzumab tetraxetan (alpha-fetoprotein), Taplitumomab paptox (CD 19), Tenatumomab (tenascin C), Teprotumumab (CD221), Ticilimumab (CTLA-4), Tigatuzumab (TRAIL-R2), TNX-650 (IL13), Tositumomab (CD20), Trastuzumab (HER2/neu), TRBS07 (GD2), Tremelimumab (CTLA-4), Tucotuzumab celmoleukin (EpCAM), Ublituximab (MS4A1), Urelumab (4-1 BB), Volociximab (integrin a5131), Votumumab (tumor antigen CTAA 16.88), Zalutumumab (EGFR), and Zanolimumab (CD4).

Citation of documents and studies referenced herein is not intended as an admission that any of the foregoing is pertinent prior art. All statements as to the contents of these documents are based on the information available to the applicants and do not constitute any admission as to the correctness of the contents of these documents.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1: Materials and Methods

Construction of Chimeric Antigen Receptors (CAR).

The CLDN6-CAR was constructed by linking the signal peptide sequence of an immunoglobulin heavy chain variable region (Gene bank number AAC18316.1) to a human CLDN6-specific single chain variable fragment (scFv) derived from the IMAB206-C46S antibody (WO20121560018). The scFv fragment is fused to human CD8a hinge and transmembrane region (Gene bank number NP_001759.3, aa 138-206) followed by human 4-1BB (Gene bank number NP_001552.2, aa 214-255) and human CD3ζ (Gene bank number NP_000725, aa 52-163, Q65K) signaling moieties. The CLDN18.2-CAR is based on the same 2nd generation CAR scaffold with substituted scFv derived from the IMAB362 antibody (WO2013174404A1). The codon-optimized and synthesized sequences (Eurofins Genomics) were cloned into the gamma-retroviral self-inactivating (SIN) vector pES.12-6 for stable overexpression in human and murine T cells under control of the short intronless version of the human elongation factor 1-alpha promoter (EFS). For in vivo imaging, CAR genes were linked to firefly luciferase (Luc) and eGFP reporter genes via 2A-splice elements (Szymczak A. L. et al., Nature Biotechnology 22, 589-594 (2004)).

Animals.

Female C57BL/6BrdCrHsd-Tyr^{c} mice, C57BL/6 mice, BALB/c were purchased from Envigo and Janvier (8-12 weeks old). Sex matched animals were used throughout the syngeneic mouse experiments. Breeding pairs of NOD.Cg-Prkdc^{scid} II2rg^{tm1Wjl}/SzJ (NSG) mice were purchased from Jackson laboratory (Bar Harbour, Me., USA). These and also congenic C57BL/6-Thy1.1 and BALB/c-Thy1.1 donor mice (Jackson Lab) were bred in the animal facility of the BioNTech SE, Germany. Studies with NSG mice were conducted with age and sex mixed animals. All experiments were performed under specific-pathogen-free (SPF) conditions and according to German animal experimentation regulations.

Cell Lines, Culture Conditions, Generation of Viral Supernatant.

Human cell lines, including ovarian cancers OV-90-5C12, SK-OV-3 and NIH-OVCAR-3, breast cancers MDA-MB-231 and MCF7, lung cancers LCLC-103H, CPC-N and COLO-699-N, SK-MEL-37 melanoma, gastric cancer 23132-87, HEK-293 embryonic kidney cells, trophoblastic cancer JAR and NEC8 embryonic testicular cancer cells were cultured under standard conditions. PA-1-SC12_A02_gfp (referred to as PA-1) and PA-1-SC12_A02_gfp_CLDN18.2_CLDN6^{-/-} (referred to as PA-1-CLDN6^{-/-} in CLDN6-CAR experiments or PA-1-CDLN18.2 in CLDN18.2-CAR experiments) are two derivatives of the human endogenously CLDN6 expressing teratoma cell line PA-1-SC12 lentivirally transduced to overexpress HLA-A*0201 and GFP. Further modifications of this cell line were lentiviral transduction for overexpression of CLDN18.2 and CRISPR/Cas9 mediated gene knock-out of CLDN6 using the following guide RNA targeting sequence (5′-3′): AA AGC GGT CAC CTT CCA CAT (Eurofins Genomics). NCI-N87-CLDN18.2 gastric cancer cell line is retrovirally transduced to overexpresses CLDN18.2. The mouse tumor cell lines LL/2-LLc1-hCLDN6 (Lewis lung cancer), CT26-mCLDN18.2 (colorectal cancer) and B16-hCLDN6 (melanoma) were generated by lentiviral transduction. For all cell lines with heterologous claudin expression (both virally transduced and RNA-transfected) the human orthologs were used, except for the CT26-mCLDN18.2 cell line.

Master and working cell banks were generated immediately upon receipt. Third and fourth passages were used for in vivo tumor experiments. Cells were tested for mycoplasma every three months.

Reauthentication of cells was not performed after receipt. 293Vec-Galv and Platinum-E cells were used for generation of GALV and MLV-E pseudotyped viral particles. Cells were transfected with TransIT-LT1 (Mirus) according to the manufacturer's instructions. Retroviral supernatants were collected 48 and 72 h after transfection and titers were evaluated using Jurkat-mCAT cells (Koste L. et al., Gene therapy 21, 533-538 (2014)). For some experiments, a stable producer clone has been used for production of retroviral supernatants (BioNTech IMFS).

RNA Constructs and In Vitro Transcription.

Plasmid templates for in vitro transcription of antigen-encoding RNAs were based on the pST1-T7-GG-hAg-MCS-2hBg-A30LA70 vector and its variants. These vectors feature 5′ and 3′ UTRs and poly(A) tails pharmacologically optimized for stability and protein translation, specifically 5′ human α-globin, two serial 3′ human β-globin UTR and a poly(A)-tail measuring 110 nucleotides in length, consisting of a stretch of 30 adenosine residues, followed by a 10 nucleotide linker sequence and another 70 adenosine. The coding sequences cloned into these vectors were the full length ORF of human Claudin-6 (NP_067018.2), Claudin-3 (NP_001297.1), Claudin-4 (NP_001296.1), Claudin-9 (NP_066192.1), Claudin-18.2 (NP_001002026.1), human CD19 (NP_001171569.1) or firefly luciferase, respectively. In one experiment RNA encoded Ovalbumin-epitope SIINFEKL (Oval) was used, which was flanked 3′ with a secretion signal and 5′ with the MHC I transmembrane and intracellular domain for optimized presentation on MHC class I and II of the transfected cell (Kreiter S. et al., J. Immunol. 180, 309-318 (2008)).

In vitro transcription and capping with β-S-anti-reverse cap analog (ARCA) were performed as described previously (Holtkamp S. et al., Blood 108, 4009-4017 (2006)).

Human Peripheral Blood Mononuclear Cells (PBMCs) and Dendritic Cells (DCs)

PBMCs were isolated by Ficoll®-Hypaque (Amersham Biosciences) density gradient centrifugation from buffy coats from healthy donors obtained from transfusion center university hospital Mainz, Germany. Monocytes were enriched with anti-CD14 microbeads (Miltenyi Biotec). Immature DCs (iDCs) were differentiated by culture medium consisting of RPMI 1640 GlutaMAX™, 50 IU/mL penicillin, 50 μg/mL streptomycin, 1 mM sodium pyruvate, nonessential amino acids, and 5% (v/v) heat-inactivated human AB serum (all from Invitrogen, Karlsruhe, Germany) supplemented with 1,000 IU/mL recombinant human (rh) GM-CSF and 1,000 IU/mL rh IL-4 (both Miltenyi Biotec) for 5 days.

Retroviral Engineering of Human T Cells

T cells were enriched from DC depleted PBMCs by magnetic separation of CD3⁺/CD28^{high} T cells using Dynabeads® Human T-Expander CD3/CD28 CTS (Life Technologies) with beads-to-CD3⁺ T-cell ratio of 3:1 and cultured in X-VIVO 15 medium (Lonza) supplemented with 5% (v/v) human serum in the presence of 450 IU/mL rh IL-7 and 50 IU/mL rh IL-15 (both from Miltenyi Biotec). Three days later CD3/CD28 beads were removed and pre-activated T cells were transduced once in the presence of 25 μg/mL Protransduzin® (Immundiagnostik AG) with GALV-pseudotyped retroviral supernatant. Cells were expanded for additional 4 days in the presence of 450 IU/mL rh IL-7 and 50 IU/mL rh IL-15 and were either directly used to assess CAR surface expression, T-cell phenotype and in vitro/in vivo-effector functions or cryopreserved.

Retroviral Engineering of Mouse T Cells

Splenocytes of either naïve C57BL/6-Thy1.1⁺ or BALB/c-Thy1.1⁺ mice were pre-activated by Dynabeads™ Mouse T-Activator CD3/CD28 (gibco) in a beads-to-CD3⁺ T-cell ratio of 1:1 in RPMI1640-GlutaMAX supplemented with 10% FBS, 1×NEAA, 1 mM sodium pyruvate, 10 mM HEPES, 50 μM b-Mercaptoethanol, 50 IU/mL Penicillin and 50 μg/mL Streptomycin in the presences of 450 IU/mL rh IL-7 and 50 IU/mL rh IL-15. 24 h after bead-activation, cells were gently spun down (1 h, 37° C., 300×g) and incubated on MLVE-pseudotyped gamma-retroviral vector pre-coated-RetroNectin-plates. After additional overnight cultivation, spin-down transduction was repeated on freshly viral particles coated plates. 72 h after initial pre-activation, Dynabeads™ Mouse T-Activator CD3/CD28 were removed from culture and cells were expanded in above mentioned medium supplied with 450 IU/mL rh IL-7 and 50 IU/mL rh IL-15 for additional 72 h. Non-transduced T cells used as control for some experiments underwent the same bead-activation and expansion procedure. Transgene expressions on transduced murine T cells were assessed via flow cytometry.

Engraftment of Tumor Cells

5×10⁶ OV90-SC12, 2-5×10⁶ NCI-N-87_hCLDN18-2, 4×10⁵ LL/2-LLC1-hCLDN6-gfp-luc or 5×10⁵ CT26-mCDLN18.2 tumor cells were injected subcutaneously in 100 μL PBS into the right back flank of mice. Prior ACT tumor-bearing were stratified using Daniels's XL Toolbox Add-in for Microsoft Excel for homogenous tumor volume distribution between treatment groups. Tumor sizes were measured with a caliper every two to three days for calculating tumor volumes using the equation (a²×b)/2 (a, width; b, length). Animals were euthanized when exhibiting signs of impaired health, when tumor ulcerated of its volume exceeded 1,500 mm³.

Adoptive Cell Transfer (ACT) and RNA-LPX Treatment

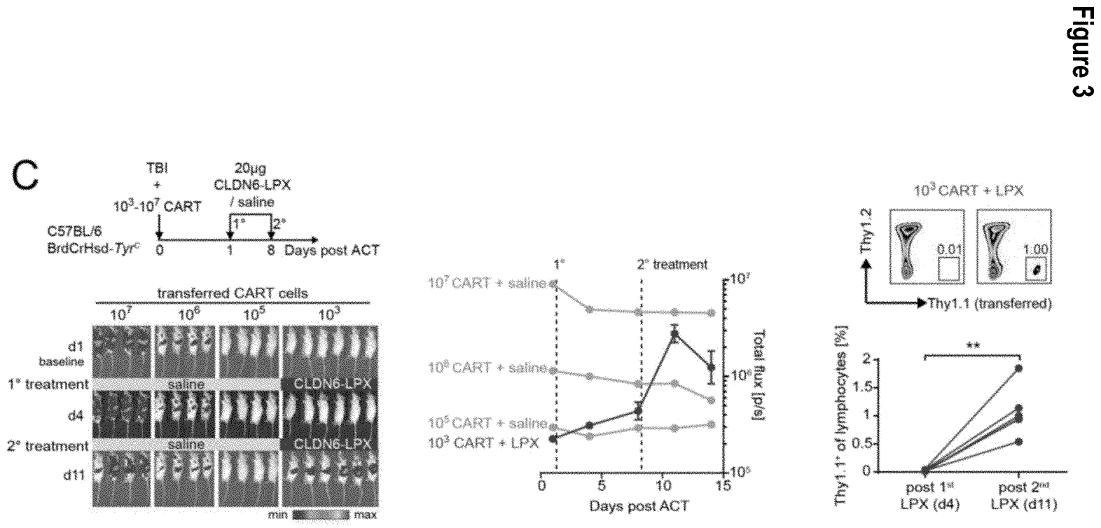

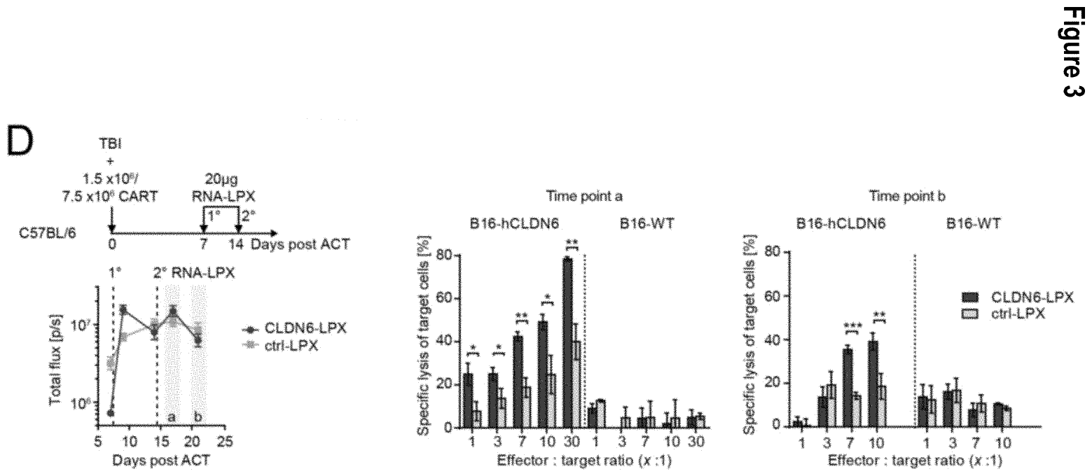

For ACT of mouse T cells, CAR-expressing Thy1.1⁺ T cells were injected intravenously into the retrobulbar venous plexus into (if not otherwise stated) total body irradiated non-tumor bearing C57BL/6BrdCrHsd-Tyr^{c} (2.5 Gy), or tumor bearing C57BL/6 (5 Gy) and BALB/c (4 Gy) donor mice, respectively. Unless otherwise stated for CAR T-cell in vivo expansion mice received 20 μg RNA-LPX. Mice treated with non-relevant antigen RNA-LPX or saline served as controls. Saline is used as synonym for PBS with the exception of FIG. 3C where 150 mM NaCl were used. For CAR T-cell in vivo expansion studies Luc-GFP co-expressing CAR T cells were used. CAR T cells without reporter gene co-expression were used to study anti-tumor efficacy.

For ACT of human T cells, thawed or freshly transduced human T cells (amount of CAR- or GFP-transgene positive T cells as indicated in figures) were injected intravenously into the retrobulbar plexus. T cells were either used immediately after in vitro activation and transduction or cryopreserved, thawed and transferred after 2 times of washing in PBS. Typically, viability of T-cell products was >90%.

Bioluminescence Imaging

Biodistribution, expansion of CAR-Luc-GFP transduced T cells, uptake and translation of Luc-RNA were evaluated by in vivo bioluminescence imaging using the Xenogen IVIS Spectrum imaging system (Caliper Life Sciences). Briefly, an aqueous solution of D-luciferin (80 mg/kg body weight; Perkin Elmer) was injected intraperitoneally. Emitted photons from live animals were quantified after 5 min with an exposure time of 1 min. Regions of interest (ROI) were quantified as total flux (photons/s, represented by color bars). The images were superimposed using the Living Image 4.0 software. For quantification of CAR T-cell expansion total flux of the indicated time point was divided by total flux at baseline.

Electroporation of IVT RNA

1 μg IVT RNA were added to cells suspended in X-VIVO 15 in a precooled 4-mm gap sterile electroporation cuvette (Bio-Rad). Electroporation was performed with an ECM 830 Square Wave Electroporation System (BTX: Colo699-N cells: 300 V, 8 ms, 1 pulse). Transfection efficiency was assessed via flow cytometry 24 h after electroporation.

Generation of Liposomal Antigen-Encoding RNA (RNA-LPX) and In Vitro Transfection of DCs

Antigen-encoding RNA was generated as described above by in vitro transcription from DNA plasmid templates, which were optimized for improved RNA-stability and translational efficiency. Full length sequences of Claudins were used including the secretion signal to ensure membrane expression in natural topology upon expression in cells. Liposome complexes were generated as described previously (Kranz L. M. et al., Nature. 534, 396-401 (2016)). Briefly, the lipid fraction contains the helper lipid DOPE in a molar ratio of 2:1 DOTMA per DOPE and particles are assembled to accomplish a charge ratio of 1.3 to 2 of cationic DOTMA and RNA. For in vitro transfection 3×10⁶ donor-matched immature DCs were incubated with CLDN6 or CLDN18.2 encoding RNA-LPX buffered in 150 mM NaCl.

Proliferation Assay

Human CAR-transduced T cells were labeled with 0.8 μM carboxyfluorescein diacetate succinimidyl ester (CFSE, Invitrogen) and co-cultured with RNA-LPX transfected DCs at 10:1 E:T ratio in round-bottom plates. Culture supernatants were harvested after 24 h for cytokine multiplex analysis. Proliferation of T cells was assessed by flow cytometry 120 h after co-culture initiation.

For in vivo proliferation analysis murine Luc-GFP co-expressing CAR T cells were labeled with 2.5 μM Cell Proliferation Dye eFluor™ 450 (CPD450, Invitrogen) prior ACT. 52 h after ACT (48 h post RNA-LPX treatment) single cell suspension of dissected spleen, ipsilateral inguinal (ing.), axillary (ax), cervical (cerv.) and contralateral ing. lymph nodes (LNs) were surface stained with anti-Thy1.1 antibody proliferation of Thy1.1⁺GFP⁺ cells were analyzed by flow cytometry.

Cytotoxicity Assays

Spheroid based cytotoxicity: 3D tumor spheroids were generated from 10⁴ PA-1-SC12_A02_gfp and PA-1-SC12_A02_gfp_CLDN18.2_CLDN6^{-/-} cell lines in ultra-low attachment 96-well plates (Costar). 48 h after seeding 10⁵ non-transduced T cells or CAR-transduced T cells were added in Gibco™ FluoroBrite™ DMEM (Life Technologies) to spheroids. Wells were imaged at 4-fold magnification and an exposure time of 300 ms to detect green fluorescence in an IncuCyte Zoom Live-content imaging system (Essen Bioscience) at 37° C., 5% CO₂. 168 h after first co-culture, T cells were challenged a second time with newly generated tumor spheroids. Data were analyzed using IncuCyte analysis software to detect and quantify the total green object integrated intensity (GCU×μm²/Image). Averages of green object counts at each time point were plotted using IncuCyte analysis software.

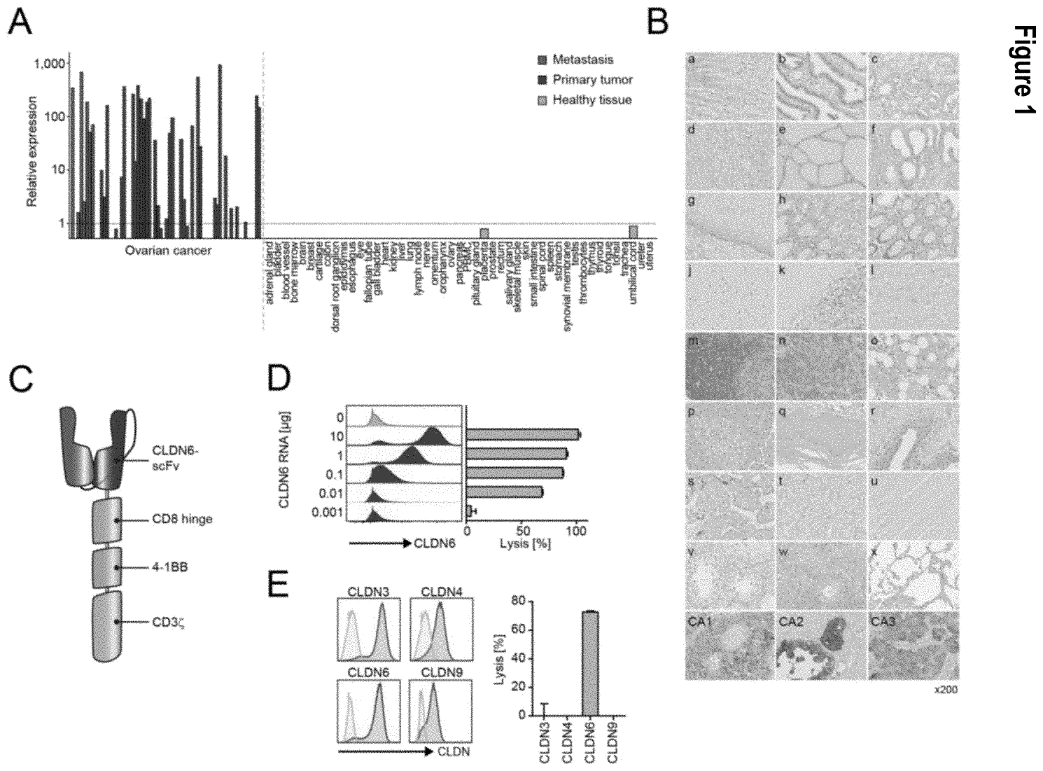

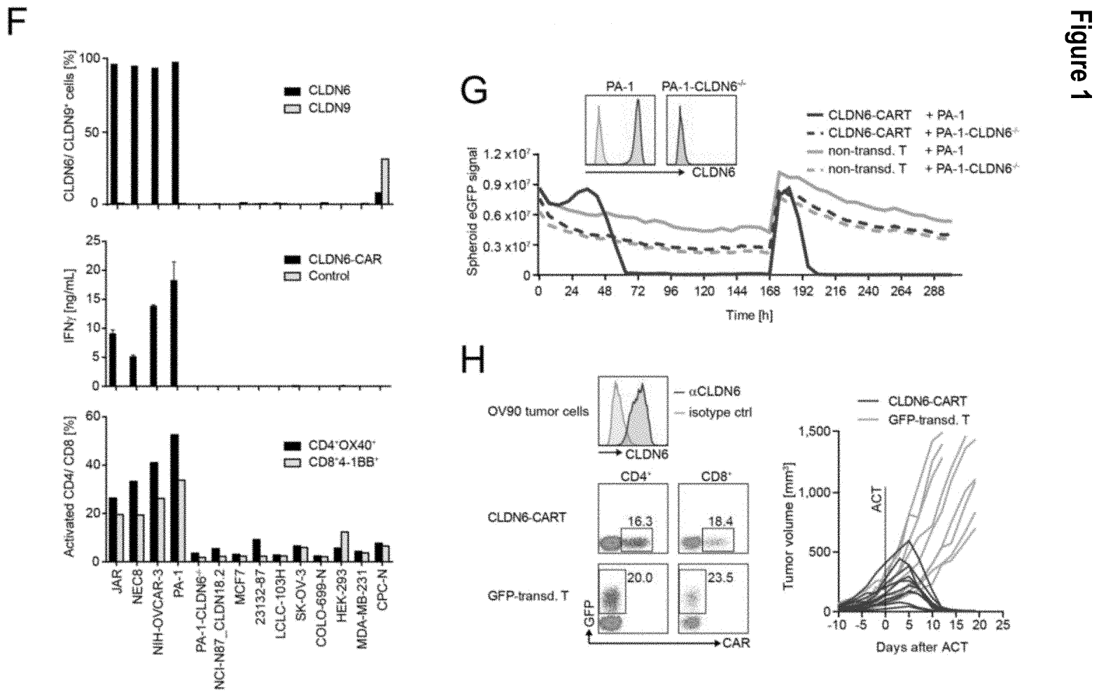

In FIG. 1D, 1E, and FIG. 3D CAR-mediated cytotoxicity was assessed using the xCELLigence system (OMNI Life Science). Cell index (CI) impedance measurements were performed according to the instructions of the supplier. 2×10⁴ human target cells (with exception of LCLC-103H, here 1×10⁴ per well) were seeded per well in E-plate 96 PET. In FIG. 3D 2.5×10³ B16-hCLDN6 or B16-F10 cell were seeded in E-plate 96 (both ACEA Biosciences Inc.). After 24-28 h either human CAR-transduced T cells or ex vivo sorted murine CAR-expressing T cells were added at indicated ratios in a final volume of 200 NL to tumor cells and monitored every 30 min for a period of up to 48 h by the xCELLigence system. The maximum CI corresponds to the minimal lysis (Lₘᵢₙ) and was assessed after either incubating target cells with non-transduced effector human T cells or murine target cells only. Percent specific lysis of human target cells was assessed after 12 or 24 h, lysis of murine target cells after 20 h of co-culture and was calculated as follows:(CI Lₘᵢₙ−CI sample)/CI Lₘᵢₙ×100.

Cytokine Multiplex Analysis

The ProcartaPlex™ 3-Plex Kit immunoassay (Invitrogen, PPX-03-MXGZFMP) consisting of pre-configured multi-analyte reagent panels of prepared magnetic beads for quantitative analysis of human IL-2, IFNγ and TNFα in culture supernatants was used after 24 h co-culturing CAR T cells with RNA-LPX transfected iDCs using Bio-Plex 2000 according to the manufacturer's instructions. High Sensitivity 5-Plex Mouse ProcartaPlex™ Panel was used to measure serum cytokine levels after different time point of RNA-LPX treatment in CLDN6-CAR T bearing C57BL/6BrdCrHsd-Tyr^{c} mice. Serum was collected and murine IFNγ, IL-2, IL-4, IL-6 and TNFα-levels were determined from samples stored at −20° C. With a few exceptions, undiluted samples were used as input of the assay. Detection levels of the used lot are the follows: IFN gamma: 0.06-260 μg/mL; IL-2: 0.12-490 μg/mL; IL-4: 0.14-560 μg/mL; IL-6: 0.70-2850 μg/mL; TNFα: 0.34-1390 μg/mL. Neither IL-2 nor IL-4 was detected. Non-detectable levels of IFNγ/IL-6/TNFα in individual samples were set to zero and were blotted in the respective graph.

Flow Cytometry

CAR surface expression on murine and human T cells was assessed by Alexa Fluor 647-coupled anti-idiotype antibodies.

Monoclonal antibodies for extracellular staining of human T cells and PBMCs, in brief CD45-PE-Cy7 (BD; clone: HI30), CD4-APC-Cy7 (BD/BioLegend; clone: SK3 or OKT4), CD4-PerCP-Cy5.5 (eBioscience; clone: RPA-T4), CD8a-BV421 (BD; clone: RPA-T8), CD137-PE (BD; clone: 4B4-1), CD134-PE (BD; clone: L106) have been used.

Surface expression of different Claudins were assessed by anti-CLDN6-DyLight650 (WO20121560018; clone: IMAB027), anti-CLDN18.2-AlexaFluor647 (WO2013174404A1; clone: IMAB362), unconjugated anti-CLDN9 (Aldevron, clone: YD-4E9), unconjugated rat IgG2b isotype control, anti-rat IgG2b-PE (both eBioscience; clone: R2B-7C3), unconjugated anti-CLDN3 (clone: 385021), unconjugated anti-CLDN4 (both R&D Systems; clone: 382321), unconjugated mouse IgG2a isotype control (BioLegend; clone: MOPC-173), unconjugated rat IgG2b isotype control (eBioscience) and affiniPure F(ab′)₂ Fragment Goat Anti-Mouse IgG (H+L)-APC (Jackson Immuno Research, polyclonal). Viability was determined using 7-AAD (Beckman Coulter), Fixable Viability Dye eFluor 506 or eFluor 780 (both eBioscience).

Monoclonal antibodies for extracellular staining of murine splenocytes or murine PBMCs, in brief: CD3-BV605 (clone: 145-2C11), CD4-APC-Cy7 (clone GK1.5), CD4-BV480 (clone: RM4-5), CD8-BV421 (all BD), CD8-eFluor506 (eBioscience), CD8-PE (all clone: 53-6.7), CD11b-FITC (clone: M1/70), CD11c-PE-Cy7 (all BD; clone: HL3), CD19-PerCP-Cy5.5 (BioLegend; clone:1D3), CD40-BV786 (BD; clone: 3/23), CD45-BV605 (BD; clone: 30-F11), CD62L-APC-Fire750 (BioLegend; clone: MEL-14), CD69-PE (BD; clone: H1.2F3), CD86-BV510 (BioLegend; clone: GL1), CD90.1-PerCP (clone: Ox-7), CD90.2-PE (BD; clone: 53-2.1), CD127-PE-Cy7 (all BD; clone: SB/199), F4/80-BV421 (BioLegend; clone: BM8), KLRG1-eFluor450 (eBioscience; clone: 2F1), NK1.1-BV786 (BD; clone: PK136), PDCA-1-PE (Miltenyi; clone: JF05-1C2.4.1) have been used.

For detection of circulating mouse or human T cells, 50 μL peripheral heparinized blood were stained with antibodies and subsequently lysed using ACK lysis buffer (gibco) or BD FACS Lysing solution (BD). Ex vivo Ki67 levels of Thy1.1⁺ lymphocytes were assessed in splenocytes at day 2 and 7 after RNA-LPX or saline treatment of C57BL6-albino mice adoptively transferred with CLDN6-CAR T cells using FoxP3/Transcription Factor Staining Buffer Set and Ki67-eFluor450 (clone: SolA15; both from eBioscience). At the same time points intracellular IFNγ staining was performed using the cytofix/cytoperm kit (BD) and an IFNγ specific antibody (eBioscience; clone: XMG1.2) after incubation of 3×10⁵ splenocytes with 1×10⁵ B16-hCLDN6 or B16-F10 WT cells respectively in the presence of 1×Protein transport inhibitor (eBioscience) for 5 h at 37° C.

Flow cytometric measurements were performed on a BD FACSCanto II flow cytometer and BD FACSCelesta respectively using the BD FACSDiva software and analysis performed using FlowJo® V10 (treestar inc.).

For cell sorting, at indicated time points splenocytes of RNA-LPX treated mice were isolated and pre-enriched separately by simultaneous magnetic depletion of endogenous T and B cells using MACS magnetic microbeads coated with CD90.2 or CD19 antibodies and MACS columns (Miltenyi Biotec). Enriched cells of each mouse were pooled per treatment group, CAR T cells (Thy1.1⁺CD8⁺GFP⁺) were then sorted on a BD FACSMelody cell sorter and used for ex vivo cytotoxicity assay.

Quantitative Real-Time PCR (qRT-PCR).

Total RNA was extracted using TRIzol/chloroform extraction protocol and clean-up of aqueous phase with RNeasy Mini Kit (QIAGEN). Subsequently, reverse transcription on 1.0 μg total RNA and the PrimeScript™ RT Reagent Kit with gDNA Eraser (Takara Bio Inc.) were used. qRT-PCR was performed using the BioMark™ HD system (Fluidigm®) with SsoFast™ EvaGreen® Supermix with Low ROX (Bio-Rad Laboratories). Samples and assays were prepared and analyzed according to the “Fast Gene Expression Analysis Using EvaGreen® on the BioMark™ or BioMark HD System” Advanced Development Protocol 37. 96.96 Gene Expression Dynamic Array IFCs were loaded using the IFC Controller HX. Primers used in the analysis: (5′-3′) CLDN6-for: ACT CGG CCT AGG AAT TTC CCT, CLDN6-rev: CAG AGG CCA TGG CGA GG, HPRT1-for: TGA CAC TGG CM AAC AAT GCA, HPRT1-rev: GGT CCT TTT CAC CAG CM GCT, TBP-for: GAG CCA AGA GTG MG AAC AGT C, TBP-rev: GCT CCC CAC CAT ATT CTG MT CT, HMBS-for: AGC CCA GCT GCA GAG AM GT, HMBS-rev: GGA TGA TGG CAC TGA ACT CC, CLDN18-for: GTG ACT GCC TGT CAG GGC T, CLDN18-rev: GGA CAC AGG AGC GCC AC (annealing temperature: 60° C.). ΔΔCT values were obtained by normalization for three housekeeping genes (Tbp, Hprt1, Hmbs) and the medium expression value derived from all analyzed tissues. In Fig. S2B 2.0 μg total RNA, oligo-dT (18×dT) primer and SuperScript II Reverse Transcriptase Kit (Invitrogen) were used for reverse transcription. Expression analysis was performed on an Applied Biosystem 7300 Real-Time PCR System Instrument and QuantiTect SYBR Green PCR Kit (QIAGEN). Primers used in the analysis: (5′-3′) CLDN6-for: CTT ATC TCC TTC GCA GTG CAG, CLDN6-rev: AAG GAG GGC GAT GAC ACA GAG (annealing temperature: 58° C.), HPRT1-for: TGA CAC TGG CAA AAC MT GCA, HPRT1-rev: GGT CCT TTT CAC CAG CM GCT (annealing temperature: 62° C.). ΔΔCT values were obtained by normalization for reference gene Hprt1 and the medium expression value derived from all analyzed tissues. For both analysis ΔΔCT values were subsequently transformed from log 2 to linear scale. The qRT-PCR data shown in FIG. 1A, were generated using healthy tissue samples obtained from commercial suppliers and post-mortem surgery samples without tumor related disease.

Expression Analysis of Published RNAseq Cohorts

RNAseq expression data was obtained for TCGA tumor (dbGaP accession: phs000178) and GTEx normal cohorts (dbGaP accession: phs000424.v4.p1). Gene expression was determined using the STAR RNA-seq aligner (Dobin A. et al., Bioinformatics (Oxford, England) 29, 15-21 (2013)) and subsequent read counting and normalization.

Immunohistochemical Staining

Formalin-fixed, paraffin-embedded (FFPE) tissue sections of surgical specimens and biopsy samples were obtained from Dr. K. Dhaene (Department of Pathology, Algemeen Stedelijk Ziekenhuis, Aalst, Belgium). Tissue micro arrays (TMAs) were purchased from Biocat (Heidelberg, Germany) or in-house collected from sacrificed mice. 3-4 μm thick tissue sections were deparaffinized, then subjected to antigen retrieval by boiling in 10 mM citric acid supplemented with 0.05% Tween-20 (pH 6.0) at 120° C. for 10 min, subsequently quenched (by 0.3% H₂O₂; 15 min) and blocked with 10% goat serum in PBS (30 min) at room temperature. Slides were incubated overnight at 2-8° C. with 0.2 μg/mL anti-CLDN6 (IBL, #18865), 1:1,000 anti-CD3 (Abcam, ab16669) 1:500 anti-Luciferase (Abcam, ab21176), 1:1,000 anti-F4/80 (Thermo Scientific, MA5-16363) or 1:500 anti-CD11c (BD Biosciences, 565227) antibodies in blocking buffer. Antibody binding was visualized with horseradish-peroxidase-labeled secondary antibodies using the polymer-based BrightVision antibodies BrightVision HRP goat-a-rabbit (Immunologic, DPVR-110HRP) together with a red substrate-chromogen solution (VectorRed; SK-4800 Vector Labs, Burlingame, USA). Sections were subsequently counter-stained with Mayer's haematoxylin (Carl Roth; T865.2) and subjected to whole slide imaging (Zeiss; AxioScan Z1).

For haematoxlyin/eosin (HE) staining the Leica ST5020 multistainer was used. After deparaffinization all slides were subjected first to Mayer's haematoxylin (Carl Roth; T865.2) thereafter to Eosin-G 0.5% (Carl Roth; X883.2), both for 4 min followed by a 5 min development in tap water. Slides were mounted and subjected to whole slide imaging (Zeiss; AxioScan Z1).

Statistical Analysis and Depiction of Data

All results are represented with mean+/−SD of technical replicates or mean+/−SEM of biological replicates. Statistical analysis for each experiment is described in the corresponding figure legend. Unpaired two-tailed Student's t-test was used for comparison of two groups. Paired t test was selected for repeated measurements of the same subjects at two different time points. One-way analysis of variance (ANOVA) was performed when more than two groups were compared. Two-way ANOVA was performed when both time and treatments were compared, and when significant (P<0.05) multiple comparisons were performed using Tukey's post-hoc test Survival benefit was determined with the log-rank test. Correlation between the number of transferred CART cells and bioluminescence was calculated by Pearson's product correlation coefficient r. All statistical analyses were performed using GraphPad PRISM 6.04. ns=not significant, *P<0.05, **P<0.01, ***P<0.001, ****P<0.0001. No statistical methods were used to pre-determine sample size for animal or other experiments.

### Example 2: An RNA Vaccine Drives In Vivo Expansion and Efficacy of CART Cells

Adoptive cell therapy (ACT) with genetically engineered T cells expressing CARs is clinically successful in patients with B-cell malignancies (Neelapu S. S. et al., The New England journal of medicine 377, 2531-2544 (2017); Maude S. L. et al., The New England journal of medicine 378, 439-448 (2018)). In patients with solid tumors, however, the efficacy of CAR T-cell therapy remains disappointing (Scarfo I. et al., Journal for immunotherapy of cancer 5, 28 (2017)). One key hurdle is the scarcity of cell surface targets with high, cancer-specific expression to allow for efficient tumor eradication and low risk of off-tumor/on-target toxicity (Morgan R. A. et al., Mol. Ther. 18, 843-851 (2010); Lamers C. H. et al., Molecular therapy: the journal of the American Society of Gene Therapy 21, 904-912 (2013); Richman S. A. et al., Cancer immunology research 6, 36-46 (2018)). We and others have recently reported cancer-associated expression of claudin (CLDN) 6, a tetraspanin membrane protein that is involved in tight junction formation (Turksen K. et al., Dev. Dyn. 222, 292-300 (2001)). To evaluate the suitability of CLDN6 as a target for CAR T-cell therapy we profiled its expression in a comprehensive set of tissues. In mice, CLDN6 has been reported to be developmentally regulated (Turksen K., Journal of cell science 117, 2435-2447 (2004)). By immunohistochemical (IHC) staining we found CLDN6 to be broadly expressed in fetal organs, but prenatally downregulated, resulting in lack of expression in most organs of adult mice (data not shown). In human, CLDN6 transcript levels were high in fetal tissues derived from stomach, pancreas, lung and kidney, but undetectable in the corresponding adult tissue samples (data not shown). In over 160 non-cancerous healthy human samples from more than 50 adult tissue types analyzed by quantitative RT-PCR, CLDN6 transcript expression was ruled out (FIG. 1A). Concordantly, CLDN6 protein was not detectable in any of the >40 tested adult human normal tissue types assessed by IHC staining (FIG. 1B). In line with previous studies (Ushiku T. et al., Histopathology 61(6):1043-56 (2012); Micke P. et al., International journal of cancer 135, 2206-2214 (2014)), high CLDN6 transcript levels were frequent in various human solid cancers such as testicular, ovarian, uterine and lung adenocarcinoma (FIG. 1A). IHC staining showed membrane expression of CLDN6 protein in these human cancers that was high and homogenous in many of the tested specimens (data not shown).

These findings indicate exquisitely tight and complete silencing of CLDN6 in human, though not in mouse, and qualify CLDN6 as a strictly oncofetal cell surface antigen with an ideal expression profile for CAR T-cell targeting (June C. H., Science (New York, N.Y.). 359, 1361-1365 (2018)). We designed a 2^{nd} generation CLDN6-CAR with a 4-1BB costimulatory domain. For the receptor domain we engineered a single-chain variable fragment (scFv) with exquisite specificity and high binding affinity to CLDN6 in the nanomolar range (FIG. 1C).

First, we characterized CLDN6-CAR-engineered human T cells in vitro. CLDN6^{neg} COLO-699N lung carcinoma cells were transfected with escalated amounts of CLDN6 RNA and assessed for killing by CAR T cells (FIG. 1D). We observed highly sensitive recognition and lysis of CLDN6-transfected target cells by the CLDN6-CAR even at the lowest target expression level.

In a similar experimental setting, we evaluated the CLDN6-CAR for cross-recognition of CLDN3, CLDN4 and CLDN9, the most closely related claudin family members, that in contrast to CLDN6 are expressed in toxicity-relevant normal tissues. The homology between the CAR-targeted first extracellular loop of CLDN6 and the corresponding amino acid sequences of these claudins is 81%, 85%, and 98%, respectively, bearing the risk of cross-reactivity and off-target toxicity of the CAR. Exclusively CLDN6-transfected target cells, but not those transfected with the related claudins, were killed demonstrating precise targeting by CLDN6-CAR T cells (FIG. 1E).

To measure cognate immune activation we co-cultured CLDN6-CAR T cells with human tumor cell lines. We found interferon-γ (IFNγ) secretion and upregulation of T-cell activation markers upon co-culture with CLDN6^{pos} targets, but not CLDN6^{neg} cells (FIG. 1F). CLDN6-CAR T cells were able to efficiently clear CLDN6^{pos} PA-1 ovarian carcinoma spheroids and to kill repetitively upon re-challenge (FIG. 1G). Deletion of CLDN6 by CRISPR/Cas9-mediated genetic knock-out (FIG. 1G top) completely abrogated CAR T-cell recognition of PA-1, further confirming high potency and target-specificity of CLDN6-CAR T cells.

Next, we studied in vivo anti-tumor activity of human CLDN6-CAR T cells in mice xenografted subcutaneously with human tumor cell lines. Of note, the binding affinity of CLDN6-CAR T cells to murine CLDN6 is 15-fold lower than to human CLDN6 and whereas human CLDN6 is strictly confined to the embryonic stage, murine CLND6 is expressed in some post-embryonic somatic tissues. NSG mice with large OV90 tumors (mean volume 168 mm³) underwent ACT with a single dose of human CLDN6-CAR T cells or control cells. Notably, all CLDN6-CAR T cell-treated mice experienced complete tumor regression within 2 weeks, while in the control group tumors progressed rapidly (FIG. 1H). Circulating CLDN6-CAR T cells were detectable in cured mice for the full observation period of up to 25 days post ACT (data not shown).

Engraftment and persistence of transferred CAR T cells are known to be critical for their clinical effect (Maude S. L. et al., The New England journal of medicine 371, 1507-1517 (2014); Kalos M. et al., Science translational medicine 3, 95ra73 (2011); Porter D. L. et al., Science translational medicine 7, 303ra139 (2015)). In hematological malignancies CAR T cells are directed against lineage antigens of B cells and encounter their targets on the host's normal and malignant B cells. These act as antigen-presenting cells (APCs) providing strong proliferation signals and promote persistence of CAR T cells (Kalos M. et al., Science translational medicine 3, 95ra73 (2011); Porter D. L. et al., Science translational medicine 7, 303ra139 (2015)).

In contrast, the frequency of CAR T cells against solid tumors typically declines rapidly (Gargett T. et al., Molecular therapy: the journal of the American Society of Gene Therapy 24, 1135-1149 (2016); Feng K. et al., Life sciences 59, 468-479 (2016); O'Rourke D. M. et al., Science translational medicine 9(399) (2017)) due to the impaired accessibility of tumor cells within solid lesions and the absence of proliferation signals when CAR T cells encounter their target in the context of an immunosuppressive tumor microenvironment. We hypothesized that expression of the CAR target in its native conformation on the surface of professional APCs in lymphoid tissues would render it accessible for cognate CAR T-cell stimulation in an optimal immune-activating environment.

Recently, we introduced intravenously administered liposomal antigen-encoding RNA (RNA-LPX) to stimulate tumor-associated T cells in the natural repertoire of cancer patients (Kranz L. M. et al., Nature 534, 396-401 (2016)). This nanoparticulate vaccine delivers antigen to APCs in the spleen, lymph nodes and bone marrow, and concomitantly initiates a Toll-like receptor-dependent type-I IFN-driven immune-stimulatory program, promoting priming and strong expansion of antigen specific T cells.

To test whether this approach could be adapted to act as a CAR T-cell Amplifying RNA Vaccine (short, CARVac), we conducted a series of experiments.

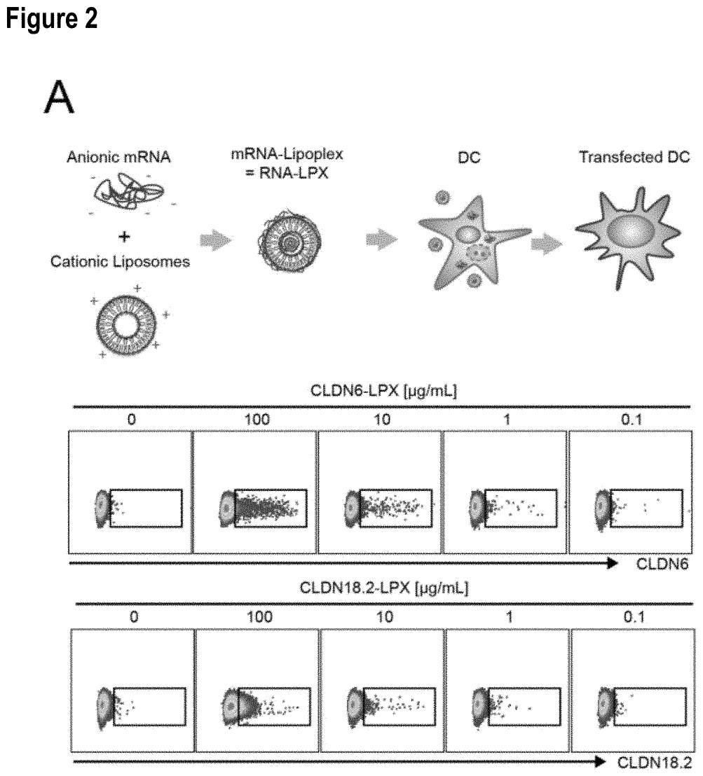

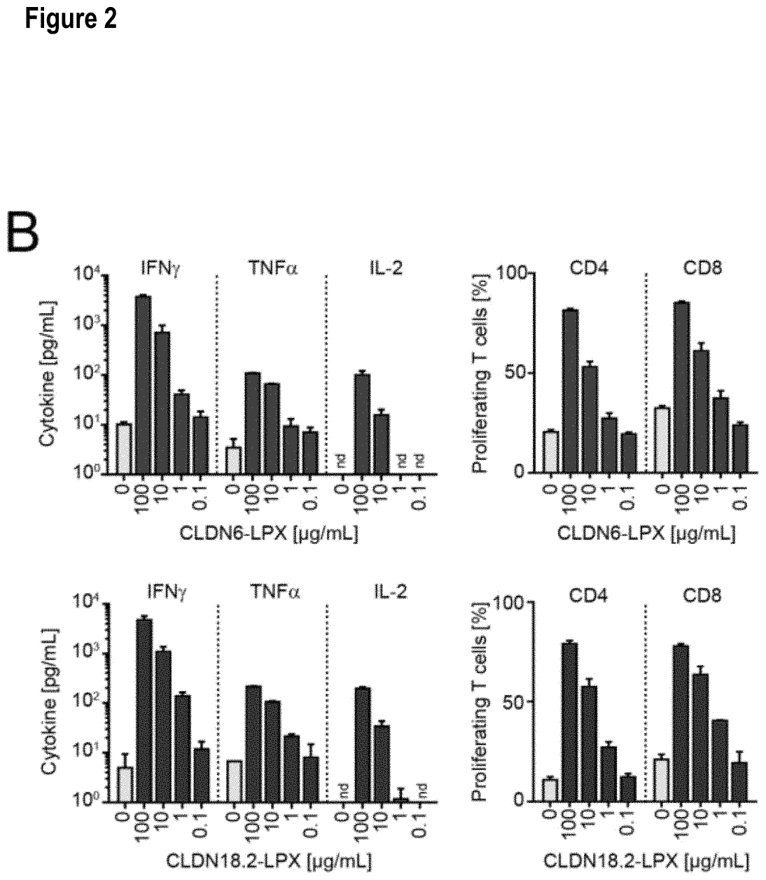

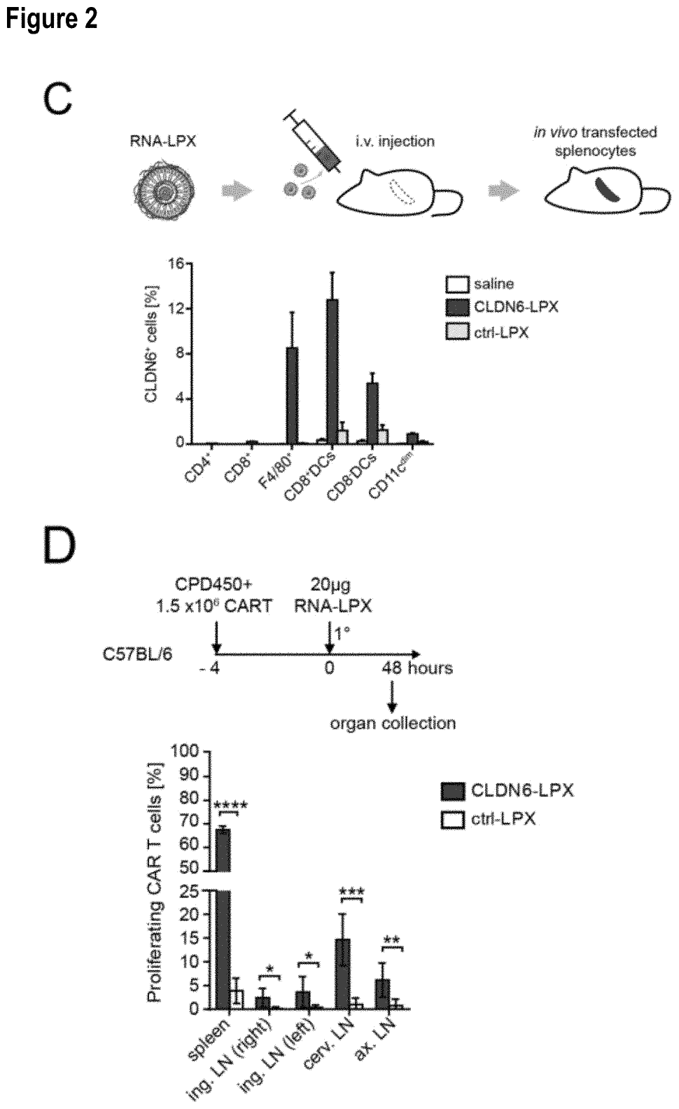

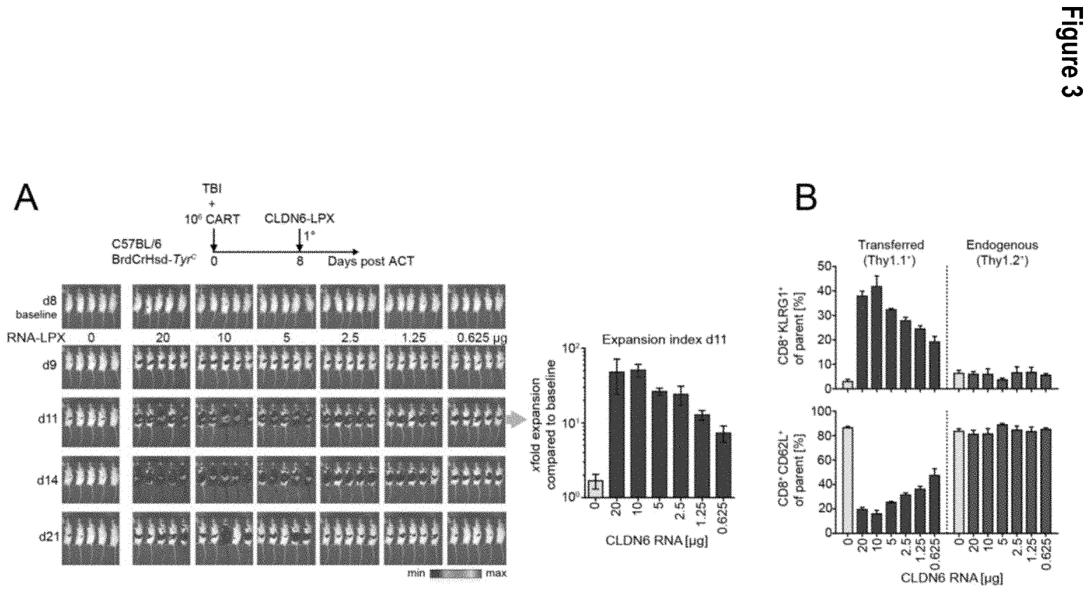

First, we tested, if CLDN6 can be natively displayed on dendritic cells (DCs) and does stimulate CLDN6-CAR T cells in vitro. We measured concentration-dependent surface expression of CLDN6 on DCs treated with different amounts of CLDN6-encoding RNA-LPX (herein CLDN6-LPX) (FIG. 2A upper panel). The resulting expression of CLDN6 on DCs induced stimulation, cytokine secretion and proliferation of co-cultured CLDN6-CAR T cells in a dose-dependent manner (FIG. 2B upper panel). When BALB/c mice were injected intravenously (i.v.) with CLDN6-LPX, CLDN6 surface expression was detected on splenic DCs and macrophages, but not on lymphocytes (FIG. 2C) confirming in vivo delivery of the CAR antigen exclusively to APCs. APCs were activated and underwent maturation (data not shown). In spleen and lymph nodes of RNA-LPX injected mice strongly activated NK, B and T cells were detected (data not shown).

Next, naïve C57BL/6 mice were engrafted with CLDN6-CAR T cells labeled with a cell proliferation dye and vaccinated with CLDN6- or control-LPX. Spleen and lymph nodes from all major body regions resected from CLDN6-LPX vaccinated but not from control treated mice displayed significantly increased proportions of proliferating CLDN6-CAR T cells, indicating body-wide functional expression of the CAR antigen within lymphoid compartments (FIG. 2D).

To assess the broader applicability of this approach, we resorted to CLDN18.2, a distantly related cancer-associated member of the CLDN family. CLDN18.2 is expressed in various high-medical need tumors, such as gastro-esophageal and pancreatic cancers (Wall S. et al., International journal of cancer 134, 731-739 (2014); Rohde C. et al., Japanese journal of clinical oncology 49, 870-876 (2019); Sahin U. et al., Clin. Cancer Res. 14, 7624-7634 (2008)). Both in human and mice, its expression in normal tissues is restricted to tight junctions of differentiated cells of the gastric mucosa, in which it is shielded. Only upon cancer-associated perturbation of the tight junction architecture, the CLDN18.2 antibody binding epitope becomes exposed (Sahin U. et al., Clinical cancer research: an official journal of the American Association for Cancer Research 14, 7624-7634 (2008)). We engineered a CLDN18.2-CAR by substituting the CLDN6-specific scFv with an anti-CLDN18.2 scFv which exhibits specific binding with similar affinity to both human and mouse CLDN18.2. CLDN18.2-CAR T cells were shown to exert similar functional features as observed for the CLDN6-CAR, including strictly antigen-specific activation and killing of tumor cells in vitro (data not shown), and complete rejection of advanced CLDN18.2^{pos} tumors in vivo (data not shown). CLDN18.2-CAR T cells co-cultured with CLDN18.2-LPX treated DCs showed cognate activation and proliferation (FIG. 2A, B lower panels).

Next, we studied the in vivo performance of the CARVac concept in a series of mouse experiments. Thy1.2⁺ C57BL/6 mice underwent total body irradiation (TBI) for lymphodepletion, were engrafted with Thy1.1⁺ CLDN6-CAR T cells co-expressing Luciferase (Luc) and GFP and subsequently vaccinated with CLDN6-LPX. In vivo bioluminescence imaging revealed that a single i.v. dose of CLDN6-LPX induced a profound expansion of circulating CLDN6-CAR T cells (FIG. 3A). The expansion correlated with the RNA-LPX dose level and was substantial at even the lowest dose of 0.625 μg RNA-LPX. Quantitative and phenotypic analysis of peripheral blood T cells in treated animals confirmed increased frequencies of Thy1.1⁺ CART cells exhibiting an activated phenotype (KLRG1^{hi}, CD62L^{low}), whereas endogenous T cells were not affected at any dose after RNA-LPX treatment (FIG. 3B).

The CAR T-cell numbers peaked 3-4 days after RNA-LPX vaccination followed by a decline, mimicking the dynamics of a physiological response of antigen-specific T cells to stimulation, with an initial expansion and subsequent retraction phase (FIG. 3A).

In another experiment, groups of mice received different dose levels of CLDN6-CAR T cells, starting as low as 10³ cells/mouse, and were either left untreated or received a CLDN6-LPX regimen shortly after ACT. In mice that did not receive CLDN6-LPX, primary CAR T-cell engraftment as quantified by bioluminescence correlated linearly with the number of adoptively transferred cells and remained stable or slowly declined over time (FIG. 3C left, middle). Notably, in mice treated according to the CARVac concept, CAR T cells were expanded irrespective of the starting dose. Actually, CLDN6-LPX mediated expansion of only 10³ CAR T cells resulted in detectable frequencies in peripheral blood (FIG. 3C right). Almost the entire adoptively transferred CAR T-cell population underwent activation and proliferation by RNA-LPX as indicated by transient upregulation of Ki67 on the majority of transferred T cells (data not shown). The RNA-LPX expanded CLDN6-CAR T cells were fully functional. As compared to CAR T cells isolated from unvaccinated mice, they produced higher levels of IFNγ (data not shown) and exerted significantly higher and strictly antigen-dependent cytolytic activity upon ex vivo co-culture with CLDN6^{pos} tumor cells (FIG. 3D).

The low-dose CAR T-cell groups benefited more from repetitive RNA-LPX treatment as indicated by increased expansion. In vivo expansion in the high-dose CAR T-cell groups stagnated after reaching high levels, suggesting a saturation threshold presumably due to T cells competing for homeostatic γc-cytokines and niches (FIG. 3C middle).

To assess the impact of repetitive RNA-LPX vaccination on long-term persistence of CAR T cells, CLDN6-CAR T cell-engrafted mice received three weekly doses of RNA-LPX followed by two further RNA-LPX administrations with longer treatment-free intervals (4 and 4.5 weeks). The first CLDN6-LPX exposure rapidly amplified CAR T cells over two orders of magnitude, subsequent weekly treatment maintained CART cells at a high level resulting in a frequency of more than 15% of total peripheral blood lymphocytes (FIG. 3E left, middle). In the longer CLDN6-LPX treatment pauses, the blood CAR T-cell frequency declined. CAR T-cell numbers did not drop to the baseline level of engraftment in unvaccinated animals, but stabilized at a ten-fold higher frequency. After each treatment-free interval CLDN6-CAR T cells could be robustly re-expanded by CLDN6-LPX, indicating memory formation of CAR T cells. Enrichment of CAR T cells with a T_{EM} (CD127⁺, CD62L^{neg}, KLRG1^{neg}) and T_{cm} (CD127⁺, CD62L⁺, KLRG1^{neg}) phenotype was confirmed by flow cytometry (FIG. 3E, right).

Cytokine release syndrome (CRS) as a clinical manifestation of excessive and prolonged secretion of pro-inflammatory cytokines in the expansion phase is the most prominent severe adverse event of CAR T cells against B-cell markers (Brudno, J. N. et al., Blood reviews 34, 45-55 (2019)). To explore the risk of CRS in conjunction with the CARVac concept, we analyzed IFNγ, IL6 and TNFα serum concentrations in gently pre-conditioned CLDN6-CAR T cell-engrafted mice after exposure to CLDN6-LPX. Remarkably, except of an early mild and transient elevation of IFNγ, no relevant inclines of pro-inflammatory cytokines were observed (data not shown) and treated mice were of normal appearance displaying regular weight gain over time (data not shown).

As repeated application of RNA-LPX and strong expansion of cytotoxic T-cell effectors might bear the risk of depletion of APCs in the lymphoid tissues, we analyzed the spleens of treated mice as the organ with the highest RNA-LPX exposure. Spleens of mice exposed to single or repetitive doses of RNA-LPX did not display any pathological alterations in spleen architecture or in appearance of red and white pulp (data not shown). Flow cytometry of the cellular composition of spleen at different time points after repetitive RNA-LPX treatment showed mild and transient reductions of CD11c⁺ DC and F4/80⁺ macrophage populations and no quantitative changes in T-cell, B-cell and NK-cell populations (data not shown). No changes were noted in the cellular distribution of APC subsets in spleen tissue sections from corresponding time points (data not shown).

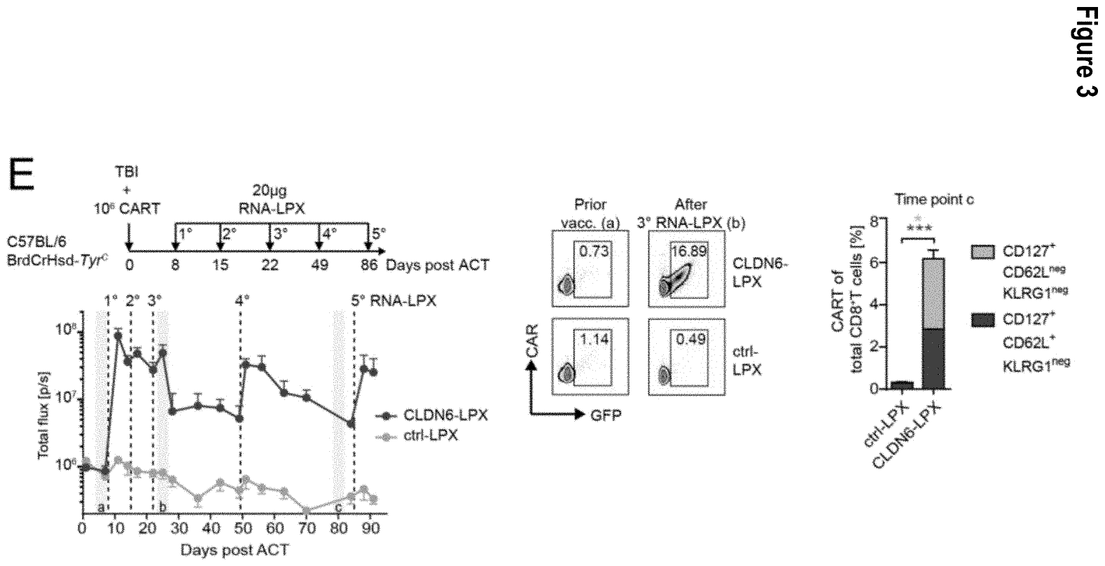

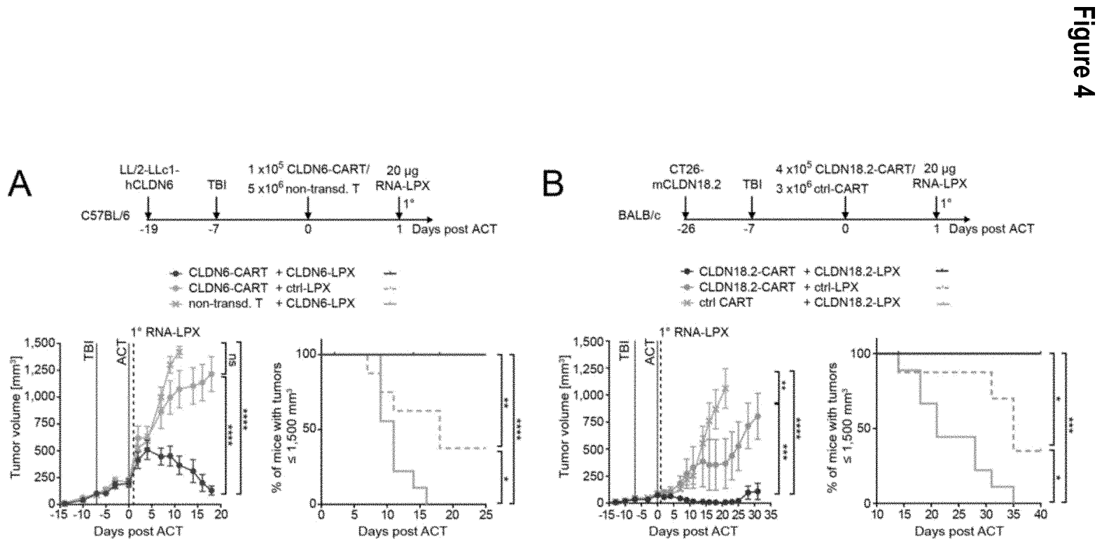

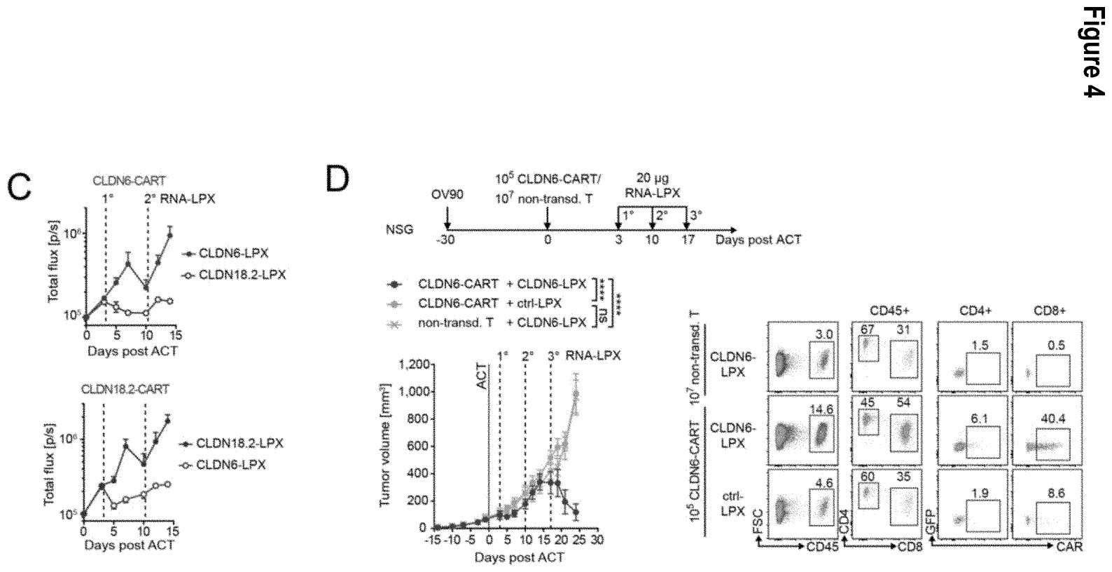

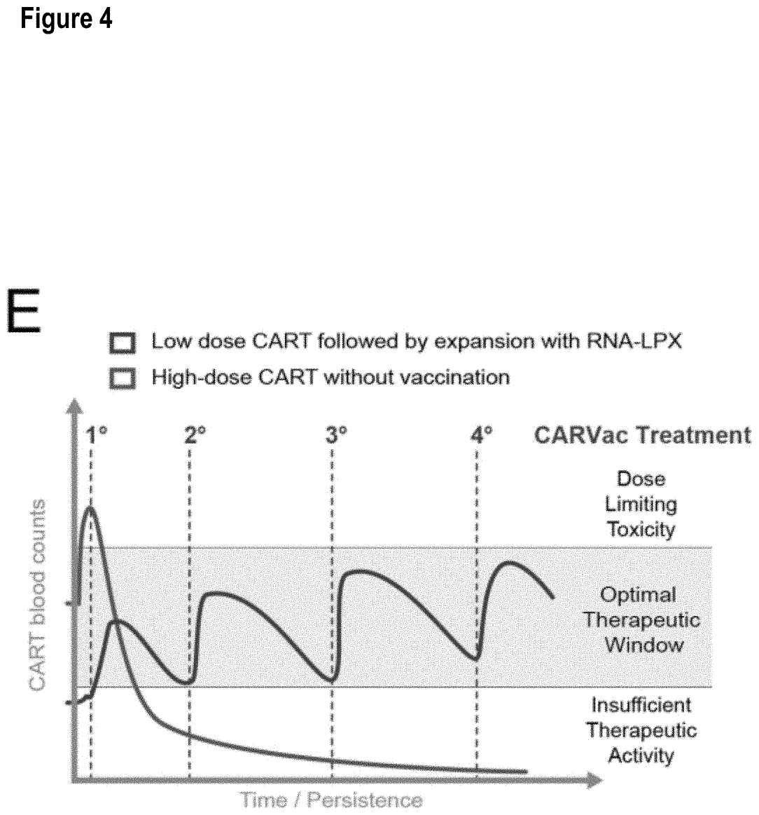

Finally, we studied the impact of RNA-LPX on the therapeutic efficacy of CAR T cells in tumor bearing mice. Lymphodepleted C57BL/6 mice with large CLDN6^{pos} LL/2-LLc1 Lewis lung tumors (mean tumor volume 209 mm³) underwent ACT with a sub-therapeutic dose of mouse CLDN6-CAR T cells followed by a single injection of CLDN6-LPX or control. Tumor control by CLDN6-CAR T cells alone was incomplete and tumor growth was only delayed. In contrast, mice receiving CART cells complemented with CLDN6-LPX application experienced complete rejection of large tumors, with a significantly higher median survival (FIG. 4A). We reproduced these findings in BALB/c mice with CLDN18.2^{pos} CT26 colon carcinomas (mean tumor volume 78 mm³) for CLDN18.2 CART cells in conjunction with a single administration of CLDN18.2-LPX, further supporting the applicability of improving the anti-tumor effect of CART cells with the CARVac concept (FIG. 4B).

For evaluation of the CARVac concept for human CAR T cells, we used the CLDN6^{pos} OV90 xenograft tumor model in NSG mice. In pilot experiments, we confirmed that NSG mice are capable of splenic uptake of RNA (FIG. 4C) and of promoting specific expansion of human CAR T cells upon repetitive RNA-LPX administration (data not shown). NSG mice bearing advanced CLDN6^{pos} OV90 tumors received a sub-therapeutic dose of 1×10⁵ CLDN6-CAR⁺ T cells followed by repetitive CLDN6-LPX or control treatment (FIG. 4D). The advanced tumors were completely rejected in CLDN6-LPX treated mice, while they rapidly progressed in the control group engrafted with the same CAR T-cell dose (FIG. 4D, left). Effective tumor control correlated with a high frequency of CAR T cells in the peripheral blood, proving their efficient in vivo expansion and improved persistence upon CLDN6-LPX vaccination (FIG. 4D, right). As with CLDN6-CAR T cells, these findings were reproduced for human CLDN18.2-CAR T cells in conjunction with CLDN18.2-LPX in a NSG mice xenograft model (data not shown).

In summary, our study establishes two key findings.

For one, our data support CLDN6 as an oncofetal cell surface antigen that is suitable for CAR T-cell targeting. In humans, the CLDN6 gene is strictly silenced in healthy adult tissues but aberrantly activated in various solid tumors of high medical need, resulting in expression of high protein levels. This, together with the feasibility of engineering a CLDN6-directed CAR of high sensitivity, precise specificity and strong potency against this surface molecule, proposes it as an ideal novel target for CAR T-cell therapy of solid cancers. Tumors without homogenous CLDN6 expression bear the risk of outgrowth of antigen loss variants. However, CLDN6 CAR T cells are strongly activated IFNγ-secreting effectors and hence, their antitumor activity is thought to drive inflammatory remodeling of the suppressive tumor microenvironment and release of endogenous tumor antigens, which together shall promote antigen-spread and counteract the rapid outgrowth of antigen loss variants (Sampson J. H. et al., Clinical cancer research: an official journal of the American Association for Cancer Research 20, 972-984 (2014)).

Second, we present the CARVac concept as an approach to improve the anti-tumor efficacy of CAR T cells. The CAR antigen is displayed in its native conformation on the surface of APCs residing in lymphoid compartments, which is the ideal setting for co-stimulation and potent expansion of T cells. Of note, it is likely that the same APCs concurrently process and present CLDN6 on MHC molecules, which may result in priming and activation of endogenous CLDN6-specific T cells. Recently, different approaches have been explored for antigen-specific expansion of CAR T cells (Berger C. et al., Cancer immunology research 3, 206-216 (2015); Slaney C. Y. et al., Clinical cancer research: an official journal of the American Association for Cancer Research 23, 2478-2490 (2017); Tanaka M. et al., Clinical cancer research: an official journal of the American Association for Cancer Research 23, 3499-3509 (2017); Ma L. et al., Science (New York, N.Y.) 365, 162-168 (2019); Wang X. et al., Clinical cancer research: an official journal of the American Association for Cancer Research 21, 2993-3002 (2015)). The CARVac approach presented here combines various distinctive features. One of the advantages of the CARVac format is that single-stranded RNA as a natural TLR-ligand combines delivery of the antigen and adjuvanticity in one molecule. Importantly, the approach does not require a re-engineering of the CAR scaffold or adaptation of T cell transduction protocols, nor depends on the cumbersome identification and characterization of peptide ligands as vaccine mimotopes. Nanoparticulate RNA-LPX is fast and inexpensive to produce for any protein-based antigen. A clinical grade manufacturing process is in place.

In ongoing clinical trials the RNA-LPX vaccine platform is used for the purpose of induction of CD4⁺ and CD8⁺ T-cell responses against a spectrum of different tumor antigens (NCT02410733, NCT02316457, NCT03815058) with early clinical data supporting the lymphoid targeting and execution of the intended mode-of-action in humans. With RNA a matched high-affinity vaccine can be generated right away and manufactured in GMP-grade for essentially any existing CAR including those directed against conformational epitopes, thus providing a truly universally applicable approach.

Our data establish the feasibility and safety of single as well as repetitive administration of CARVac for tunable expansion of engineered T cells. RNA-LPX stimulated CAR T cells are superior over non-stimulated ones with regard to cytokine response and cytolytic activity upon antigen recognition. They form memory T cells and persist at higher frequencies. The CARVac approach not only improves the engraftment of transferred CAR T cells but also enables therapeutic tumor control at lower CAR T-cell doses (FIG. 4E).

The expansion, retraction and re-stimulation kinetics of CAR T cells mediated by RNA-LPX mimic the physiological process T cells undergo upon antigen-specific priming and boosting. That the magnitude of CAR T-cell expansion depends on RNA-LPX dose, allows control of the levels of circulating CART cells, and titration of CAR T-cell frequencies within an optimal therapeutic window.

In addition to lack of suitable targets and fast decline of CAR T cells in the circulation, other barriers for efficacy of CAR T cells in human solid cancer exist, including tumor antigen heterogeneity, impaired T-cell trafficking and extravasation to tumor sites, exhaustion and an immunosuppressive microenvironment. Accomplishing to maintain optimally stimulated CAR T cells within a therapeutic window may provide a good foundation for overcoming those constraints as well.

### Example 3: RNA-LPX Mediated Vaccination is Also Applicable for In Vivo Expansion and Enrichment of TCR-Modified T Cells

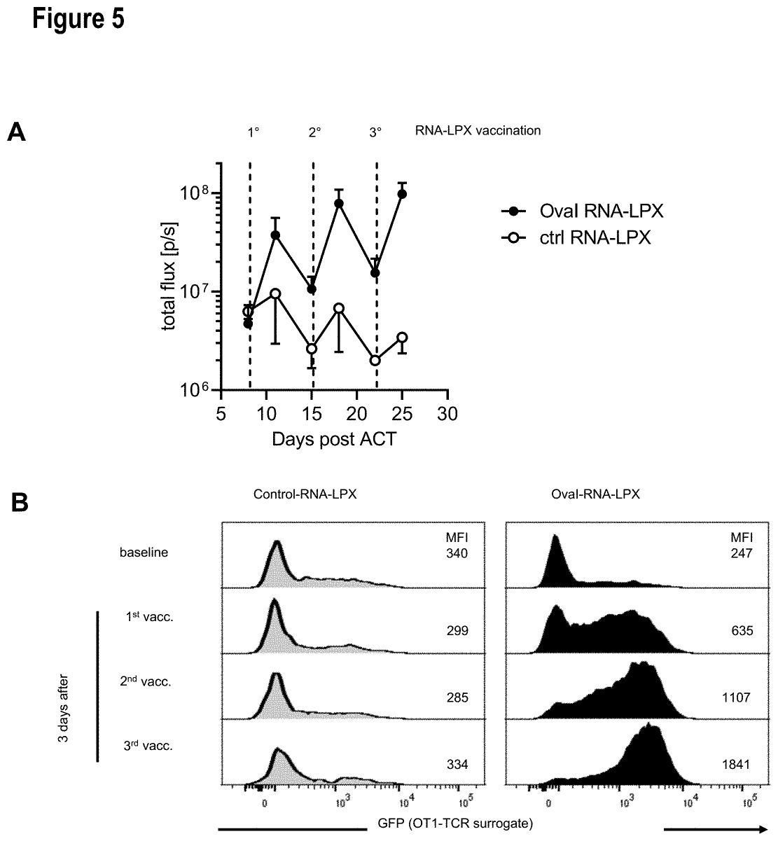

The clinical success of adoptively transferred tumor reactive T cell therapy has been also positively correlated with the persistence of those cells in vivo (Robbins et al. (2004) J Immunol. 173(12):7125-30, Huang et al. (2005) 28(3):258-67). Beside CAR T cells expansion, we analyzed whether in situ antigen exposure could also enhance the persistence of adoptively transferred TCR-modified T cells in vivo. Therefore, luciferase co-expressing OT1-TCR transduced murine T cells were adoptively transferred into mildly irritated (2.5 Gy) mice followed by repetitive administration of RNA-LPX encoding either or a control antigen. Expansion and enrichment of the OT1-TCR transduced T cell population were sequentially monitored by bioluminescence imaging (FIG. 5A) and flow cytometry. (FIG. 5 B).

OT1-TCR-modified T cells are able to expand in vivo after repetitive vaccination with liposomally formulated TCR antigen. OT1-TCR-modified T cells bioluminescence signals decreased over time in the control group while antigen specific restimulated OT1 TCR T cells bioluminescence signal enriched after every Oval-RNA-LPX treatment FIG. 5A. In accordance to bioluminescence data, flow cytometric analysis of GFP-expressing OT1-TCR T cells resulted in an enrichment of transferred OT1-TCR T cells (GFP acts as marker for transferred OT1-TCR expressing T cells) in peripheral blood during repetitive stimulation. In contrast to control-RNA-LPX treated group where the mean fluorescence of GFP-expressing cells in mice remained constant (FIG. 5 B). These data demonstrate that RNA-LPX technology also supports adequate TCR-modified T cell activation by providing natural co-stimulation in situ, which can lead to similarly enrichment of TCR-modified T cells in vivo.

1. A method for treating a subject comprising:
(i) providing sub-therapeutic amounts of immune effector cells genetically modified to express an antigen receptor to the subject, and
(ii) administering to the subject an antigen targeted by the antigen receptor, a polynucleotide encoding the antigen, or a host cell genetically modified to express the antigen.

2. The method of claim 1 which is a method of inducing an immune response in said subject.

3. The method of claim 2, wherein the immune response is a T cell-mediated immune response.

4. The method of claim 2 or 3, wherein the immune response is an immune response to a target cell population or target tissue expressing an antigen.

5. The method of claim 4, wherein the target cell population or target tissue is cancer cells or cancer tissue.

6. The method of claim 4, wherein the cancer cells or cancer tissue is solid cancer.

7. A method for treating a subject having a disease, disorder or condition associated with expression or elevated expression of an antigen comprising:
(i) providing sub-therapeutic amounts of immune effector cells genetically modified to express an antigen receptor, the antigen receptor being targeted to the antigen associated with the disease, disorder or condition or cells expressing the antigen associated with the disease, disorder or condition, to the subject, and
(ii) administering to the subject an antigen targeted by the antigen receptor, a polynucleotide encoding the antigen, or a host cell genetically modified to express the antigen.

8. The method of claim 7, wherein the disease, disorder or condition is cancer and the antigen associated with the disease, disorder or condition is a tumor antigen.

9. The method of claim 7 or 8, wherein the disease, disorder or condition is solid cancer.

10. A method for treating a subject having a solid cancer associated with expression or elevated expression of a tumor antigen comprising:
(i) providing immune effector cells genetically modified to express an antigen receptor, the antigen receptor being targeted to the tumor antigen or cells expressing the tumor antigen, to the subject, and
(ii) administering to the subject an antigen targeted by the antigen receptor, a polynucleotide encoding the antigen, or a host cell genetically modified to express the antigen.

11. The method of claim 10, wherein the immune effector cells genetically modified to express an antigen receptor are provided to the subject in sub-therapeutic amounts.

12. The method of any one of claims 1 to 11, wherein the immune effector cells genetically modified to express an antigen receptor are provided to the subject by administering the immune effector cells genetically modified to express an antigen receptor or by generating the immune effector cells genetically modified to express an antigen receptor in the subject.

13. A method for treating a subject comprising:
(i) generating immune effector cells genetically modified to express an antigen receptor in the subject, and
(ii) administering to the subject an antigen targeted by the antigen receptor, a polynucleotide encoding the antigen, or a host cell genetically modified to express the antigen.

14. The method of claim 13 which is a method of inducing an immune response in said subject.

15. The method of claim 14, wherein the immune response is a T cell-mediated immune response.

16. The method of claim 14 or 15, wherein the immune response is an immune response to a target cell population or target tissue expressing an antigen.

17. The method of claim 16, wherein the target cell population or target tissue is cancer cells or cancer tissue.

18. The method of claim 17, wherein the cancer cells or cancer tissue is solid cancer.

19. A method for treating a subject having a disease, disorder or condition associated with expression or elevated expression of an antigen comprising:
(i) generating immune effector cells genetically modified to express an antigen receptor, the antigen receptor being targeted to the antigen associated with the disease, disorder or condition or cells expressing the antigen associated with the disease, disorder or condition, in the subject, and
(ii) administering to the subject an antigen targeted by the antigen receptor, a polynucleotide encoding the antigen, or a host cell genetically modified to express the antigen.

20. The method of claim 19, wherein the disease, disorder or condition is cancer and the antigen associated with the disease, disorder or condition is a tumor antigen.

21. The method of claim 19 or 20, wherein the disease, disorder or condition is solid cancer.

22. A method for treating a subject having a solid cancer associated with expression or elevated expression of a tumor antigen comprising:
(i) generating immune effector cells genetically modified to express an antigen receptor, the antigen receptor being targeted to the tumor antigen or cells expressing the tumor antigen, in the subject, and
(ii) administering to the subject an antigen targeted by the antigen receptor, a polynucleotide encoding the antigen, or a host cell genetically modified to express the antigen.

23. The method of any one of claims 13 to 22, wherein the immune effector cells genetically modified to express an antigen receptor are generated in the subject in sub-therapeutic amounts.

24. The method of any one of claims 1 to 23, which is a method for treating or preventing cancer in a subject.

25. The method of claim 24, wherein the cancer is solid cancer.

26. The method of claim 24 or 25, wherein the cancer is associated with expression or elevated expression of a tumor antigen targeted by the antigen receptor.

27. The method of any one of claims 1 to 26, wherein the antigen receptor is a chimeric antigen receptor (CAR) or T cell receptor (TCR).

28. The method of any one of claims 1 to 27, wherein the polynucleotide encoding the antigen is RNA.

29. The method of any one of claims 1 to 27, wherein the host cell genetically modified to express the antigen comprises a polynucleotide encoding the antigen.

30. The method of any one of claims 1 to 29, wherein the immune effector cells genetically modified to express an antigen receptor comprise a polynucleotide encoding the antigen receptor.

31. The method of any one of claims 1 to 30, wherein the immune effector cells are T cells.

32. A kit comprising:
(i) immune effector cells genetically modified to express an antigen receptor or a polynucleotide encoding an antigen receptor, and
(ii) an antigen targeted by the antigen receptor, a polynucleotide encoding the antigen, or a host cell genetically modified to express the antigen.

33. The kit of claim 32, wherein the polynucleotide encoding an antigen receptor is useful for genetic modification of immune effector cells to express an antigen receptor.

34. The kit of claim 32 or 33, further comprising instructional material for use of the kit in the method of any one of claims 1 to 31.


SEQUENCE LISTING (15 sequences)

SEQ ID NO: 1 - DNA, 20 bp, Artificial Sequence
  Oligonucleotide
aaagcggtcaccttccacat

SEQ ID NO: 2 - DNA, 21 bp, Artificial Sequence
  Oligonucleotide
actcggcctaggaatttccct

SEQ ID NO: 3 - DNA, 17 bp, Artificial Sequence
  Oligonucleotide
cagaggccatggcgagg

SEQ ID NO: 4 - DNA, 21 bp, Artificial Sequence
  Oligonucleotide
tgacactggcaaaacaatgca

SEQ ID NO: 5 - DNA, 21 bp, Artificial Sequence
  Oligonucleotide
ggtccttttcaccagcaagct

SEQ ID NO: 6 - DNA, 22 bp, Artificial Sequence
  Oligonucleotide
gagccaagagtgaagaacagtc

SEQ ID NO: 7 - DNA, 23 bp, Artificial Sequence
  Oligonucleotide
gctccccaccatattctgaatct

SEQ ID NO: 8 - DNA, 20 bp, Artificial Sequence
  Oligonucleotide
agcccagctgcagagaaagt

SEQ ID NO: 9 - DNA, 20 bp, Artificial Sequence
  Oligonucleotide
ggatgatggcactgaactcc

SEQ ID NO: 10 - DNA, 19 bp, Artificial Sequence
  Oligonucleotide
gtgactgcctgtcagggct

SEQ ID NO: 11 - DNA, 17 bp, Artificial Sequence
  Oligonucleotide
ggacacaggagcgccac

SEQ ID NO: 12 - DNA, 21 bp, Artificial Sequence
  Oligonucleotide
cttatctccttcgcagtgcag

SEQ ID NO: 13 - DNA, 21 bp, Artificial Sequence
  Oligonucleotide
aaggagggcgatgacacagag

SEQ ID NO: 14 - DNA, 21 bp, Artificial Sequence
  Oligonucleotide
tgacactggcaaaacaatgca

SEQ ID NO: 15 - DNA, 21 bp, Artificial Sequence
  Oligonucleotide
ggtccttttcaccagcaagct